(19) United States
(12) Patent Application Publication
(10) Pub. No.: US 2024/0175090 A1
(43) Pub. Date: May 30, 2024

(54) STRATIFICATION METHOD THAT DETERMINES THE PROGNOSIS OF BREAST CANCER AT A PERSONALIZED LEVEL

(71) Applicant: The Research Foundation for The State University of New York, Albany, NY (US)

(72) Inventors: Vladimir Kuznetsov, Syracuse, NY (US); Leszek Kotula, Syracuse, NY (US)

(73) Assignee: The Research Foundation for The State University of New York, Albany, NY (US)

(21) Appl. No.: 18/508,755

(22) Filed: Nov. 14, 2023

Related U.S. Application Data
(60) Provisional application No. 63/425,006, filed on Nov. 14, 2022.

(51) Int. Cl. C12Q 1/6886

(52) U.S. Cl. CPC C12Q 1/6886; C12Q 2600/118; C12Q 2600/158

(57) ABSTRACT

The present disclosure provides the ABI1-based seven-gene prognostic signature that predicts survival of metastatic breast cancer patients. The present disclosure reveals that ABI1 is a prognostic metastatic biomarker in breast cancer. The present disclosure further provides that lung metastasis is associated with an ABI1 gene dose and specific gene expression aberration in primary breast cancer tumors and indicates that targeting ABI1 may provide a therapeutic advantage in breast cancer patients.

### CROSS REFERENCE TO RELATED APPLICATION

This application claims the benefit of priority from U.S. Provisional Application No. 63/425,006, filed on Nov. 14, 2022, the entire contents of which are incorporated herein by reference.

### FIELD OF THE DISCLOSURE

The present disclosure relates to methods for assessing metastasis risk of cancer (e.g., breast cancer) based on analysis in tissue sample and an interdisciplinary approach involving genetics, wet lab biochemistry, structural biology, pharmacology, histology, and bioinformatics. More particularly, this disclosure reveals the significant role of ABI1 (Abelson interactor protein 1) gene and a subset of the WAVE (Wiscott-Aldrich Verprolin homologous protein) complex genes in the context of breast cancer progression and metastatic process.

### BACKGROUND ART

Breast cancer is the most commonly diagnosed non-cutaneous cancer in American women, causing an estimated 200,000 deaths and over 40,000 new diagnoses each year [1]. Despite current treatment modalities that combine surgical intervention, radiation, and adjuvant chemotherapy, many patients relapse after years of treatment and present with metastatic and often incurable diseases. Metastasis of breast tumors accounts for the majority of breast cancer-related deaths [2]. Thus, there is an urgent need to identify novel molecular targets for the development of new treatments against breast cancer.

Although the overall number of deaths from breast cancer decreased over the last decade for low-risk tumors, mortality remains high for patients with invasive metastatic disease. Several molecular targets for breast cancer have remained the mainstay for therapy including the estrogen receptor (ER), epidermal growth factor receptor (EGFR), human EGFR-2 (Her2, neu), and phosphatidylinositide 3-kinase (PI3K). Targeted therapeutics against these factors are approved for clinical use or are undergoing clinical trial testing in breast cancer patients. The recent FDA approval of Trastuzumab Deruxtecan (T-DXd) for HER2-low tumors bring excitement for treatment of larger patients' population with previously dismal prognosis. The continued identification of mechanisms leading to invasive breast cancer is of vital importance, as it will aid in the discovery of new pathways and targets for therapeutic intervention.

### SUMMARY OF THE DISCLOSURE

This disclosure demonstrates the metastasis driver role of ABI1 in breast cancer tumor progression using the PyMT (polyoma middle T antigen) mouse model and clinical data from breast cancer patients. The bioinformatics analyses reveal the significant role of human ABI1 and a subset of the WAVE complex genes in the context of breast cancer progression and metastatic process. The present disclosure identifies the multigene survival prognosis signature comprised of ABI1 and six other genes: BRK1 (BRICK1 subunit of SCAR/WAVE actin nucleating complex), CYFIP1 (cytoplasmic FMR1-interacting protein 1), CYFIP2 (cytoplasmic FMR1-interacting protein 2), and WASF3 (these genes are part of WAVE complex members); and RAC1 and NDEL1 genes, which are upstream interactors and regulators of the WAVE complex. The present disclosure defines the role of individual ABI1-signature genes in the metastatic process in vivo using the breast cancer mouse model and allows identification of the metastatic pathway for diagnosis and treatment target development.

Therefore, one aspect of the present disclosure is directed to a method of determining the personalized risk of metastasis of breast cancer and the risk of survival in a subject who has or had breast cancer. The method comprises (a) obtaining a tissue sample from the subject; (b) measuring gene expression profiles of ABI1, BRK1, WASF3, CYFIP1, CYFIP2, RAC1 and NDEL1 genes in the tissue sample; (c) comparing the gene expression profiles of above-mentioned seven genes in the tissue sample from a high-risk primary tumor from the subject with metastatic or death outcomes with the expression profiles of these genes from a low-risk primary tumor, thereby comprising a seven-gene prognostic signature; (d) determining the risk of the subject using data-driven grouping (DDg) methods, gene expression data and statistically weighted voting grouping (SVWg) algorithms; and (e) stratifying the subject in a high, moderate, or low risk group based on the determining step.

In some embodiments, a computer readable medium having stored thereon a computer program which, when executed by a computer system operably connected to a gene or protein expression assay system configured to measure an expression signal of a plurality of genes in a tissue sample obtained from a subject, causes the computer system to perform a method of calculating a cut-off value by a method comprising: (a) fitting said measured expression signal of ABI1, BRK1, WASF3, CYFIP1, CYFIP2, the WAVE complex members, and also RAC1 and NDEL1 as independent variables, interpreting the expression signals and calculating a p-value using a data-driven grouping (DDg) method; (b) constructing a seven-gene prognostic signature using a statistically weighted voting grouping (SVWg) algorithm and input data provided by the data-driven grouping (DDg) method; and, stratifying the subject in a high, moderate, or low risk group based on the seven-gene prognostic signature.

In some embodiments, ABI1 is an independent prognostic metastatic biomarker in breast cancer.

In some embodiments, a metastasis is associated with ABI1 gene dose and specific gene expression aberrations in primary breast cancer tumors.

In some embodiments, the subject is a mammal. In some embodiments, the mammal is a human.

In some embodiments, the tissue sample is a mammary gland tissue sample or a breast cancer tissue sample or its derivatives found in the human body at any stage of the disease.

In some embodiments, ABI1 gene is highly or abnormally expressed, thereby representing a therapeutic target, as defined in a metastatic ABI1/PyMT mouse model system, wherein ABI1 gene downregulation reduces metastatic burden in lungs.

### BRIEF DESCRIPTION OF THE DRAWINGS

FIGS. 1A-1D. ABI1 expression alteration is associated with copy number alteration (CNA) and high-aggressive basal-like breast cancer. Box Plots: (A) Putative ABI1 DNA copy number alteration (CNA) drives ABI1 transcription level in subpopulations of primary breast cancer patients [42]. The gene expression, CNA, tumor samples and clinical datasets representing 1904 primary breast cancer samples were downloaded from METABRIC dataset. CNA categorization is the following: shallow deletion: 1(n=166), diploid: 2(n=1554), gain: 3(146), amplification: 4 (n=38). One-way ANOVA test (Statistica 13) showed significant differences in the ABI1 expression between the groups and also in the entire cohort (p<1.00E-9). Furthermore, the transcription level of ABI1 is highly significant and positively correlated with CNA ((r-0.338; p<1.00E-6; estimated by Spearman). (B) ABI1 transcription level positively correlated with histologic grades (Univariate and bivariate linear regression models testing shown significance at p<1.00E-6), however (C) negatively correlated with ER status. Bivariate linear regression models (Statistica 13) showed that both expression ABI1 expression level and CNA are significant (r=−0.278; p<1.0.00E-6 and r=−0.207, p<1.00E-6 respectively), however, the ABI1 expression provides a major contribution in the bivariate linear regression function). Correlate coefficients in (B) and (C) were calculated by Kendall. (D) ABI1 over-expression is associated with basal-like and claudin-low breast cancer subtypes and aggressiveness of breast cancer scoring also by histologic grade. PAM50 (Basal-like, HER2(+), luminal B, luminal A, normal-like) and claudin-low subtypes were ranked-order according to the trend of decreasing of ABI1 expression. One-way ANOVA test (Statistica 13) showed significant differences in the ABI1 expression between basal-like, claudin-low subtypes and other subtypes (p<1.00E-6). ABI1 expression in the HER2 subtype was significantly higher than in luminal B or luminal A tumor subtypes (p<1.00E-6) and higher but less significant than in the normal-like tumor subtype (p=001). A negative trend in the ABI1 expression across rank-ordered tumor subtypes was mostly defined by relative over-expression of Basal-like and claudin-low tumor subtypes; it was highly significant (One-Way ANOVA; p<1.00E-9; Statistica 13).

FIGS. 2A-2F. ABI1-based prognostic signature predicts disease-free and metastatic-free survival risks. The disease-free survival (DFS) and disease metastasis-free survival (DMFS) of patients stratified based on the ABI1-associated signature derived by survival prognostic analysis method is shown using Kaplan-Meier survival curves for Rosetta (A, C) and MetaData cohorts (B, D). The Wald statistic p-value and Hazard Ratio (HR) associated with the partitioning of the patients into distinct risk groups are also shown. The method computationally categorizes each covariate (expression level of a gene) as a binarizing risk factor and stratifies each patient according to the multivariate expression pattern of the genes included in the signature (Table 2). In panels (A), (B), (C) and (D): black color line=‘low-risk’, red=‘intermediate risk’, blue=‘high-risk’ groups. Panels (E) and (F) represent the overall survival (OS) time functions for the patients with metastasis detected after diagnostic and following surgical treatment. The black color line is associated with the group of patients with relatively better disease outcomes, while the red color is associated with patients with poor disease outcomes. The tables at the bottom of plots show the number of patients who survived in the predicted groups more than the given time point.

FIGS. 3A-3G. ABI1 loss does not impact the long-term development of healthy mouse mammary glands. (A) Whole-mount analysis of the inguinal mammary gland stained with Carmine Alum reveals no gross changes in gland anatomy at 5, 7, or 12 weeks of age after CRE-mediated deletion of ABI1. Morphometry of whole mounts reveals a significant increase in the number of terminal end buds in homozygous ABI1 null glands (B); however, this does not affect the elongation of the ductal tree (C) or the number of ductal branches (D). Scale bar, 5.0 mm. (E) Histological staining of mammary gland sections reveals no changes in tissue organization after CRE-mediated loss of ABI1. Scale bar, 100 μm. (F) Immunostaining of mammary sections using markers for luminal epithelial cells (CK8) and myoepithelial cells (CK14) reveals sustained organization of the ductal epithelium in both control and ABI1 null mammary glands. Scale bar, 50 μm. Error bars indicate SEM. (* indicates p<0.05, t-test; n=5 animals/genotype). (G) WB blotting analysis indicates enhanced expression of Abi1 in mammary epithelium of Abi1(fl/fl) PyMT mice vs. Abi1 floxed mice Abi1 (fl/fl). Each lane represents one mammary gland (Abi1 fl/fl) or tumor [PyMT: Abi1(fl/fl)], (n=3 mice).

FIGS. 4A-4D. Abi1 KO severely impacts WAVE complex gene expression dynamics. (A) Western blot analysis of primary mammary tumors from Abi1 KO PyMT mice shows significant depletion of ABI1 protein, but only in the homozygote Abi1 null is there significant upregulation of ABI2 protein as indicated by densitometry (B). Each lane represents one mammary tumor isolated from one animal of that genotype. Error bars indicate SEM. (p<0.05, t-test; n=3 animals/genotype). (C) Analysis of primary tumor histology reveals no significant changes in tumor grade between controls and Abi1 knockouts, (p>0.05, t-test; n≥5 mice per genotype of age between 20-22 weeks were used for analysis, Table 9). Error bars indicate SEM). (D) Immunostaining with antibodies against WAVE complex proteins supports the findings that ABI2 is upregulated only in ABI1 null breast tumors. WAVE1 retained its low expression, while WAVE2 was concomitantly depleted with ABI1, in agreement with WB data, above. 20× magnification; inset, 40× magnification, Scale bar, 50 μm.

FIGS. 5A-5E. Primary tumor growth kinetics analysis indicates Abi1 gene dose effect in heterozygous mice. (A) Primary tumor latency in PyMT animals is not significantly affected upon Abi1 KO. The X-axis of a panel (A) represents latency time comparison of the tumors in four treatment conditions defined on the upright corner of the panel (Abi1 fl/fl Cre−, n=14 mice; Abi1 fl/fl Cre+, n=20 mice, Abi1 fl/wt Cre−, n=16; Abi1 fl/wt Cre+, n=16 mice). (B-E) Treatment effects of Abi1 disruption (fw Cre(+) vs fw Cre(−) and tumor kinetics of tumor size in heterozygous or homozygous mice. Graphical tools of Statistica-13 were used. Each plot on panels (B-E) shows tumor size at seven-time points (w0, w1, w3, w3, w4, w5 and w6) (for Abi1 fl/fl Cre+, or Cre−, n=13 mice were used; for Abi1 fl/wt Cre+, or Cre−, n=11 mice were used). The line connects start (Cre(−)) with the endpoint (Cre(+)) tumor size datasets allowing the comparison of tumor kinetic observations to be easily followed; mean values of tumor size are linked by direct lines at the same detection time point. Wilks lambda statistics, Fisher test were used for estimation of treatment significance. Panels (B) and (C) represent a visualization of the treatment effect (Cre(−) v.s. Cre(+)) of Abi1 on tumor size in observed time points. Vertical bars indicate 0.95 intervals, CI. An effective decomposition method of Statistica-13 was used. The primary tumor size comparison in fast growing mouse groups shows exponential growth kinetics. (Table 11). To compare gene dosage effects within heterozygote and homozygote groups, mean values in 7 observed time points were compared (Table 11). The results showed that in the cases of fast kinetics datasets, differences between the paired sample mean values were not significant for homozygote (t-test, p>0.15) but significant for heterozygote state (t-test, p=0.017) (Table 11).

FIGS. 6A-6G. Abi1 gene knockout reduces metastatic burden in heterozygous and homozygous mice. Representative tumor kinetics of primary (panels A-B) vs metastatic tumors (panels C-D). Panel (A) Comparison of the primary tumor volume kinetics in Abi1 homozygous KO mouse (fl/fl; Cre+) (G209, data: red triangle; best-fit function: red line) and the control Abi1 (fl/fl Cre−) mouse (G184, data: blue circle; best-fit function: blue line). (B) Comparison of the primary tumor kinetics of Abi1 KO heterozygous (fl/wt Cre+) mouse (G251, data: pink triangle; best-fit function: pink line) and the Abi1 control (fl/wt; Cre−) mouse (G202, data: green circle; best-fit function: green line). Kinetics of mean values (a and b) were fitted by exponential curve ƒ(t; a, b)=(a−b)*exp(at), where t is time, constant a is the rate of cell population growth and constant (a−b) is the initial tumor population size. Each kinetic dataset includes seven time points (Table 11). The estimated parameters in Abi1 fl/fl Cre (−) tumors: a=0.77+/−0.159, t-test, p=0.0047, b=0.2+/−0.678, t-test, p>0.1 and in Abi1 fl/fl Cre (+) a=0.60+/−0.153, t-test, p=0.0039), b=0.1+/−0.586, t-test, p>0.1. Estimated parameters in Abi1 fl/wt Cre (−) tumors: a=0.79+/−0.091, t-test, p=0.001), b=−1.00+/−0.964, t-test, p>0.1, and in Abi1 fl/wt Cre (+) a=0.589+/−0.110, t-test, p=0.0031, b=−0.30+/−0.66, t-test, p>0.1. According to these results, differences between mean values of the tumor sizes in the studied groups in time are not significant. While primary tumor volume kinetics was not significantly different in these mice vs. their corresponding controls, (A, homozygous ABI1 KO vs. control) and (B, heterozygous KO vs. control), the difference in metastatic tumor burden of the same mice within each mouse genotype was significant (C) and (D). Panels (C) and (D) show the frequency distributions of a lung metastatic foci size in the heterozygous and homozygous mice, which primary tumors kinetics showed on panels (A) and (B) respectively. Each Y-axis value shown in the histograms (C)-(D) represents a count of metastatic foci within a metastatic size normalized interval (a bin). The bin was defined by rounding the metastatic size divided by 1000 to the nearest integer, and the number of metastatic foci in each bin was counted. Based on our findings, the metastases size frequency distribution in the lung has skewed form with the long right tail. To provide a visualization of such frequency distribution, log₁₀-log₁₀ it plot was used. The same color was used for dots of the empirical distributions and the fitting function lines, as was indicated in the figures. Such empirical frequency distribution was modeled and parameterized using the shifted log-normal distribution function:

ƒ(x;y₀,x₀,a,b)=y₀+a*exp(−0.5*(ln(x/x₀)/b²),

where x is the node size and y₀, x₀, a, b unknown parameters. The parameters were estimated using the non-linear curve fitting option of SigmaPlot-13 software. Datasets and detailed results of the parameterization of this function are presented in Table 10. (E-F) show histogram bar plots for the distribution of the average number of metastases foci size in the lungs of Abi1 KO mice in comparison to their genetic controls. X-axis indicates binning for every 5000 μm² metastasis colony area size, with bin 1 representing 0-5000 μm² and bin 21 representing 100001 μm² and larger; Y-axis: count of the samples within given binning interval (+/−SEM). The size stratification of individual metastatic colonies shows that mice lacking ABI1 still have relatively small metastatic colonies, but they grow slowly or/and stay at dormant state and appear unable to establish macro metastases when compared to the controls (p<0.001; Wilcoxon signed-rank test). Lung metastasis quantification was performed following fixation, paraffin embedding and sectioning: three 5 μm sections (sectioned every 50 μm) were collected from each mouse (Abi1 fl/fl, Cre−, n=7; Abi1 fl/fl, Cre+, n=6; Abi1 fl/wt, Cre−, n=6; Abi1 fl/wt, Cre+, n=6; animals per genotype, age 18-22 weeks), were stained with Hematoxylin and Eosin, and imaged using Omnyx digital pathology scanner (GE Healthcare). Images were quantified using ImageJ software (NIH). Results of panels (E) and (F) support the results presented in (C) and (D). (G) Histological staining of representative lung sections reveals severely diminished metastasis upon deletion of the Abi1 gene. Scale bar, 1 mm. Inset, 4× magnification.

FIGS. 7A-7C. Annotation discrepancies and cluster analysis. The examples of false-positive (probe 1) and multiple isoforms mapping (probe 2), top and bottom respectively (A). Here, instances are shown of incorrection annotation in the Rosetta probes that have been excluded by the pipeline. PCA analysis of the microarray expression dataset pre-batch effect correction and pos-batch effect correction, left and right respectively, utilizing ComBat (B) and KS-weighted mean (C) Batch correction method implementation. The data distributions for the 1-st and the 2-nd PCA components were visualized. Here, it is shown that before batch effect correction, outlier batches and high variation of data points are observed. However, the batch correction leads to better overlapping of the points and batches. Utilizing ComBat and KS-weighted mean correction provides similar results.

FIGS. 8A-8D. Example of KS-weighted means batch effect correction and its effect on survival analysis. Distributions of ABI1 expression values for each group pre-batch effect correction and post-batch effect correction, left and right respectively (A). Here, increased consistency of ABI1 expression values between groups is shown. Survival analysis of survival time and event death of original dataset and KS-corrected dataset including all patients left and right respectively (B). Survival analysis of survival time and event death of original dataset and KS-corrected dataset in only patients with metastasis left and right respectively (C). Survival analysis of metastasis time and metastasis event of the original dataset and KS-corrected dataset in only patients with metastasis, left and right respectively (D). In the case of (B) and (C), KS-weighted means batch effect correction results in better significance. For (D), KS-weighted means batch effect correction provides results that adhered to findings in the literature while the original dataset did not.

FIGS. 9A-9B. Risk-predicting ability of individual members of the ABI1 based prognostic signature in disease-free survival (DFS). Kaplan-Meier survival curves depict the survival associated with patients stratified into low-risk and high-risk groups according to the signature in the Rosetta (A) and MetaData (B) datasets. In all plots, black color is associated with low-risk and red color is associated with high-risk.

FIGS. 10A-10B. Risk-predicting ability of individual members of the ABI1 based prognostic signature in metastasis disease-free survival (MDFS). Kaplan-Meier survival curves depict the survival associated with patients stratified into low-risk and high-risk groups according to the signature in the Rosetta (A) and MetaData (B) datasets. In all plots, black color is associated with low-risk and red color is associated with high-risk.

FIGS. 11A-11D. Commonly used clinical variables are insufficient for robust patient risk stratification. Kaplan-Meier survival curves for the Rosetta dataset were stratified based on (A) estrogen receptor (ESR) status (red: positive vs. black: negative; Log-rank p=0.0022) and (B) lymph node status (red: positive vs. black: negative; Log-rank p=0.52). Kaplan-Meier survival curves for the Rosetta dataset were stratified based on (C) estrogen receptor (ESR) status (red: positive vs. black: negative; Log-rank p=0.0.85) and (D) lymph node status (red: positive vs. black: negative; Log-rank p<0.001).

FIG. 12. Survival predictive analysis (RFS time) at transcription and protein level suggests a pro-oncogenic role of ABI1 in BCa progression and outcome. (A) Microarray mRNA expression dataset of the 3951 BC patients suggests a pro-oncogenic role of ABI1 in BC progression and outcome. p=0.0001, Log-rank statistics test; FDR=5%, Median survival time (months): low expression: 216.7; high expression: 185.2. Expression cut-off value: 746, expression range: 121-4621. Dataset source and method:https://kmplot.com/analysis/index.php?p=service&cancer=breast. (B) Protein level analysis support ABI1 as a survival prognostic marker in BC patient samples. Survival prediction analysis (Data: GEO/NCBI GSE39004) (2): http://kmplot.com/analysis/index.php?p=service&cancer=breast_protein

FIGS. 13A-13D. The implementation of 2D-DDg survival prediction to Rosetta data (DFS and DMFS). Panels (A) and (C) show the K-M function plots for low- and high-risk groups defined by ABI1 expression paired with the expression of the other 6 genes (as potential interaction partners) for DFS and DMFS respectively. Panels (B) and (D) provide a visual presentation of bi-bivariate distributions of the expression data in the case of DFS and DMFS respectively. The scatter plots give a visual representation of the separation of patients according to the individual cut-off values associated with each gene in the synergistic gene pairs, for Rosetta (B) and MetaData (D) datasets. Each circle represents a tumor sample; circles with red outlines are associated with relatively high risk and circles with black outlines are associated with lower-risk survival outcome groups. In (A) and (C) the red color indicates the survival time of the high-risk group, while the color black indicated the survival function of the low-risk group. In panels (B) and (D), the points colored with red correspond to patients in the high-risk group, and the black points refer to patients in the low-risk group.

FIG. 14. Schematic representation of the role of ABI1, BRK1, WASF3, CYFIP1, CYFIP2, RAC1 and NDEL1 genes in metastasis.

### DETAILED DESCRIPTION OF THE DISCLOSURE

The present disclosure provides the ABI1-associated gene expression signature, which predicts the disease metastasis free survival (DMFS) of patients with primary breast cancer. The signature includes a subset of WAVE complex genes (ABI1, BRK1, WASF3, CYFIP1, CYFIP2), and the direct interactors of WAVE complex (RAC1 and NDEL1). ABI1 is an essential component of the signature. To model the role of Abi1 in breast cancer tumor progression and metastasis, Abi1 gene expression was conditionally depleted in the mammary epithelium of PyMT breast cancer mice using the mammary-specific Cre recombinase mouse. The analysis shows that Abi1 knockout (KO) mice, both with homozygous and heterozygous deletion had more diverse tumor growth kinetics compared to the controls. In KO animals, a significant proportion (between 54% to 64%) of the primary tumors grew slower or not at all. However, the number of identified metastatic foci in lung and their size were significantly reduced in both homozygous and heterozygous KO mice; with the more significant metastasis suppression effect observed in the former. These results indicate that Abi1 gene dosage in primary tumors is critical for the progression of metastasis in breast cancer. Western blotting analysis of primary tumors supports that ABI2 protein expression is increased in animals with homozygous deletion of Abi1. Collectively, the analyses utilizing both human breast cancer gene expression data and genetically engineered Abi1 knockout breast cancer mouse models support the critical role of ABI1 and ABI1-based gene prognostic signature as novel biomarkers of breast cancer metastases.

The critical role of actin polymerization in breast tumor progression and invasion is well established, but the underlying mechanisms remain to be elucidated. Candidate mechanisms of tumor progression involving actin include cell-matrix interactions, invadopodia formation, and increased cell motility, which can all be attributed to increased actin polymerization in invading cells [3, 4]. The WAVE complex is a heteropentameric nucleation-promoting factor of F-actin polymerization and comprises WAVE proteins (1/2/3), Abelson interactor (1/2/3), SRA1/CYFIP1, NAP1, and BRK1/HSPC300 [5-7]. These proteins are encoded by genes WASF(1,2,3), ABI (1/2/3), CYFIP(1,2), NCKAP1, and BRK1 respectively [7]. The WAVE regulatory complex in response to RAC1 activation has been proposed to act as a regulator of cell motility by promoting ARP2/3-dependent actin polymerization at the leading cell edge [8, 9]. Importantly, increased levels of ARP2/3 and WAVE2 are correlated with an increased risk of invasive breast cancer [10].

The integrity and activity of the WAVE complex are reliant on the presence of all complex members; the loss of any single constituent can lead to altered cell phenotypes [11]. Upstream pathway signaling partners of WAVE complex such as RAC1 [12, 6, 13] and NUDEL modifies its activity [14]. Abelson interactor 1 (ABI1) is crucial for WAVE complex stability and regulation of specific actin-dependent processes such as cell motility and adhesion, macropinocytosis, and embryonic development [15-17]. Constitutive Abi1 loss results in murine embryonic lethality [16]. ABI1 is an adaptor protein that promotes phosphorylation of substrates, such as WAVE2, by ABL kinase and has also been shown to be important for capping of F-actin filaments, thus highlighting its regulatory role in cellular homeostasis and actin turnover [18]. WAVE1 and WAVE2 have differential roles in actin polymerization output resulting in distinct effect on actin meshwork at the plasma membrane [19] [20].

In cancers, WAVE complex's molecular composition is dynamic and can be represented by distinct molecular sub-complexes due to deregulation of component levels [11, 7, 14]. Furthermore, several cell context-dependent WAVE/ABI1 sub-complexes can form and exhibit distinct functions activated and maintained through different mechanisms [11, 7, 20, 14]. For instance, enhanced levels of WASF3 gene expression could promote cancer cell invasiveness and are associated with the highly aggressive breast cancer subtypes [21, 22]. However, recent studies also demonstrate potential tumor suppressor function of Wasf3 upon overexpression in PyMT breast cancer cells [22] thus indicating heterogeneity of WAVE3-based complex signaling through differential effect on actin cytoskeleton and cell proliferation [23, 24].

WAVE complex dysregulation in cancer provides input into cell cycle progression and warrants the study of its role in breast cancer [25]. Although the specific molecular mechanism has yet to be uncovered, WAVE2 was linked to regulation of cell cycle progression through RAC1, Arp2/3 and ARPIN. Upregulation of the Arp2/3 subunit, ARPC1B, is associated with very poor metastasis-free survival of breast cancer patients, but inhibition of ARP2/3 prevents cycle progression through RAC1 transformation [7, 25].

Alterations in ABI1 expression have been associated with tumor initiation and progression in human cancers, thus indicating that ABI1 protein levels must be tightly regulated in cells. ABI1 dysregulation has been implicated in several cancers, such as breast, brain, colon, stomach, ovarian, and prostate cancers [26-29]. Notably, the role of ABI1 in cancer is not always the same; in some cancers, such as PMF, glioblastoma and prostate cancer, ABI1 expression is downregulated [26, 27, 30, 31], whereas in breast cancer ABI1 expression is enhanced [32], thus suggesting the tissue and disease-involving pathway specificity of the role of ABI1 in oncogenic transformation and indicating the importance of mechanistic studies. The important role of ABI1 in breast cancer has been established in clinical samples. Previously, immunohistochemical studies of over 900 human breast tumor samples showed that ABI1 overexpression is positively correlated with poor survival and a shorter relapse time in human breast cancer patients [32]. Indeed, the analysis revealed that invasive breast tumors have higher ABI1 protein expression than poorly invasive tumor samples and that increased ABI1 protein levels are significantly correlated with earlier recurrence and shortened survival. These findings have been supported by xenograft models of highly aggressive breast cancer cells (MDA-MB-231) lacking ABI1, which were unable to grow into large tumors in immunocompromised mice [33]. Taken together, previous data suggest that ABI1 plays a driving role in the progression of metastatic breast cancers [33, 34, 32].

Several in vitro studies have shown the impact of ABI1 in driving breast cancer cell motility, division, and invasiveness; however, its exact role during in vivo tumor initiation, progression, and metastasis remains to be elucidated. The present disclosure provides the impact of Abi1 loss on mammary tumor initiation and progression using the polyoma middle T (PyMT) breast cancer mouse model. The PyMT antigen is a transmembrane scaffolding protein with key tyrosine residues that, upon phosphorylation, can activate signaling pathways involved in cell proliferation and survival (e.g., PI3K/AKT and MAPK), making it a reliable model for aggressive breast tumor formation [35]. The PyMT breast cancer model has been well characterized and recapitulates human breast cancer pathology, especially that of the triple-negative subtype [36].

High levels of ABI1 have been associated with the risks of metastasis of primary tumors and breast cancer mortality, as well as associated with the metastatic phenotype of human breast cancer cell lines in vitro [37, 38, 3, 34, 32]. The deficiency of ABI1 has been shown to reduce cell migration and invasiveness of aggressive breast cancer cells and is associated with activity in pathways such as PI3 kinase/AKT and SRC [33, 32].

The present disclosure determines the mechanisms involving the protein ABI1 in the progression of metastatic disease and to develop novel strategies for breast cancer treatment. The present disclosure leads to novel treatment paradigms in breast cancer and contribute to personalized medicine and survival prediction.

The term “subject” as used herein refers to any mammal. The mammal can be any mammal, although the methods herein are more typically directed to humans. In specific embodiments, the subject includes a human cancer patient. In some embodiments, the subject has breast cancer or has an elevated risk of metastasis of breast cancer.

The term “cancer”, as used herein, includes any disease caused by uncontrolled division and growth of abnormal cells, including, for example, the malignant and metastatic growth of tumors. The term “cancer” also includes pre-cancerous conditions or conditions characterized by an elevated risk of a cancerous or pre-cancerous condition.

The term “metastasis” as used herein refers to the movement or spreading of cancer cells from one organ or tissue to another. Cancer cells usually spread through the blood or the lymph system. If a cancer spreads, it is said to have metastasized.

The term “stratification” as used herein, refers to classification of something into different groups. In specific embodiments, stratification of the subjects includes stratifying the subject in a high, moderate, or low risk group based on the risk of metastasis of breast cancer.

Breast cancer is heterogenous disease characterized by biomarker hormone receptors/HER2 expression. Estrogen Receptor (ER) and Progesterone Receptor (PR) are key hormones, whose expression distinguish breast cancer subtypes. In addition to hormone expression, the presence of HER2 is critical for initial prognosis and therapy selection. The presence/absence of ER, PR, and/or HER2 characterize Luminal A, B, and HER2 positive breast cancer subtypes, respectively and guide therapy. Luminal A are ER/PR+ and HER2 negative, with Ki67+ in less than 20% of cells. They comprise 50-60% of all breast cancers. Luminal A tumors are treated with third generation hormonal aromatase inhibitors (AI), estrogen receptor inhibitors such tamoxifen or selective ER regulators. Luminal B tumors have high K167+, are ER/PR+ and can be HER2+ or HER2−. The latter responds better to neoadjuvant chemotherapy, while HER2 positive tumors are sensitive to trastuzumab. Across Luminal A and B, and HER2+ tumors, the metastatic phenotype is associated with high ABI1, but its expression is the highest in Basal-like/TNBC phenotype.

Triple Negative Breast Cancer (TNBC) is the deadliest subtype of breast cancer comprising 10-20% of all and characterized by its metastatic phenotype. TNBC tumors do not express ER, PR or HER2 resulting in them also being known as triple negative breast cancer (TNBC). Majority of TNBC tumors are basal-like, with reported 60-90% overlap between the subgroups but the terms TNBC and basal-like are not interchangeable. Basal-like tumors are characterized by genes present in normal breast myoepithelial cells such as cytokeratins CK 5, CK17, P-cadherin, nestin, caveolin 1-2, CD44 and EGFR and have an increased incidence of p53 and BRCA1 mutations, which result in high genomic instability, tumor aggressiveness, and poor prognosis. Basal-like tumors appear at an early age having a large tumor size and high histological grade. They also have a high mitotic index, and the pattern of metastatic relapse is aggressive, which mainly occurs in visceral organs such as lungs, central nervous system and the lymph nodes. The ABI1 gene is overexpressed or amplified in basal-like breast cancer. ABI1 gene has regulatory elements that bind ER and AR. Moreover, ABI1 is a co-regulator of the AR-mediated transcription. ABI1 vs ER/AR are negatively correlated in breast cancer. This leads to the hypothesis that upregulation of ABI1 is associated with development of hormone receptor independence during breast cancer progression. The present disclosure determines how ABI1 regulates hormone receptors during breast cancer progression.

Androgen receptor is a novel target in TNBC. “Luminar AR” (LR) is a subtype of TNBC with better prognosis but resistance to neoadjuvant chemotherapy. There are four molecular subtypes of TNBC based on their transcriptional profiles: two basal subtypes (BL1 and BL2), a mesenchymal (M) subtype lacking immune cells, and a luminal androgen receptor subtype (LAR) that is enriched in AR expression and follows its transcription program. Initial results from clinical trials indicated potential clinical benefit for targeting the AR pathway in breast cancer. Retrospective studies in patients demonstrated that LAR tumors are less responsive to standard chemotherapy than other TNBC tumors, underscoring the need for identification of novel therapeutic strategies. The present disclosure defines the role of ABI1 as a therapeutic target in LAR. Claudin-low subtype of breast cancer tumors are characterized by low expression of genes involved in tight junctions and intercellular junctions such as claudin, occludin and E-cadherin. Claudin-low phenotype is associated with EMT, and markers of stemness and immune response. These tumors are difficult to treat. These tumors are also characterized by high ABI1 expression and metastatic phenotype.

The present disclosure provides the new viable target for metastatic breast cancer, namely ABI. The present disclosure shows that targeting ABI1 abrogates lung metastasis in the preclinical Abi1/PyMT mouse model. Moreover, using gene dose experiments, the present disclosure demonstrates the differential effect on metastasis: disruption of one copy of the Abi1 gene produced less significant effect than complete inactivation of Abi1 gene, thus clearly demonstrating the cause-effect relationship of Abi1 gene in promoting metastasis. Identification of Abi1 as clear target for therapy is significant. In addition, the novel mouse model of breast cancer metastasis is established as resource for research community.

The present disclosure determines breast cancer tumor sensitivities based on ABI1 levels, which leads to establishment of ABI1 as predictive biomarker for personalized treatment. The stratification of patients based on the Abi1 levels leads to development of diagnostic modalities in future treatments of breast cancers and leads to viable drug development.

The present description is further illustrated by the following examples, which should not be construed as limiting in any way. The contents of all cited references (including literature references, issued patents, and published patent applications as cited throughout this application) are hereby expressly incorporated by reference.

### EXAMPLES

### Example 1. Reannotation and Legacy Comparison Microarray Datasets

The updated and re-annotated Rosetta microarray dataset [39] and the Metadata dataset [40, 41] were used for the statistical testing and survival prediction analyses. The Metadata dataset is comprised of Uppsala and Stockholm data cohorts, which totals 249 samples (Affymetrix U133A, U133B) [40, 41]. Rosetta expression microarray dataset of 295 primary breast cancer samples has been downloaded [39] and re-processed. Probe sequences (60 bp) obtained from the Rosetta dataset were aligned using NCBI's command line blastn program with the following arguments: -reward 2-penalty-3-word_size 11-gapopen 5-gapextend 2.

Coordinates with the most significant e-value were used for each sequence. Ensembl GRCh38.p13 was used to annotate each probe's given genome coordinates. RefSeq gene symbols were used to annotate probes that were not annotated by Ensembl and contained RefSeq IDs. A total of 32439 expression data points are present with 24479 unique probes (GSE159956).

The newly updated Rosetta probe set annotation was compared to the original probe set annotation. A total of 11847/24479 (48.4%) of the probe's gene symbols exactly matched the original gene symbol. A total of 804/24479 (3.2%) probes that have identical gene symbols were on the opposite strand of the given gene. Of the 12632/24479 (51.6%) unique probes that were not an exact match, there were instances where the original set either had a false negative, a false positive, or an alternative gene symbol was used (FIG. 7). An example of a false negative was probe ‘Contig44690_RC’. In the original probe set annotation, there was no gene symbol for this probe. However, it was found that the gene symbol for this probe was PTEN, which was confirmed with the UCSC genome browser.

A total of 7375/24479 (30.1%) probes matched this characteristic. An example of a false positive was probe ‘Contig52193_RC’. In the original probe set annotation, there was a gene symbol present for this probe. However, it was found that the location of the probe was neighboring the gene body rather than overlapping the gene body. A total of 92/24479 (0.003%) probes matched this characteristic. The remaining percentage of probes that did not exactly match were either instances where the official gene symbol had been updated or instances where a single probe mapped to a locus containing multiple genes. An example of an updated gene symbol was probe ‘NM_017546’. In the original probe set annotation, this probe mapped to gene C40; however, the method mapped this probe to CNOT11. Using Gene Cards, it was found that CNOT11 and C40 were the same genes. An example of a probe that mapped to multiple genes was probe ‘NM_006340’. This probe mapped to both BAIAP2 and AATK. The original probe annotation only linked this probe to BAIAP2. The original probe set annotation was improved by providing gene symbols for a significant portion of false negatives (FIG. 7). Additionally, increased consistency of ABI1 expression values was shown between groups following KS-weighted means batch effect correction (FIG. 8).

### Example 2. Characterization of ABI1 Expression, Copy Number Alterations and Associations with Breast Cancer Clinical Data

To analyze ABI1 expression, copy number alterations and associations of these characteristics with breast cancer clinical data, The Molecular Taxonomy of Breast Cancer International Consortium (METABRIC) was used ([42] observed in cBioPortal for Cancer Genomics). ABI1 profiles from 1904 breast cancer patients including microarray expression, copy number variation, clinical and cancer samples were downloaded and analyzed.

### Example 3. Survival Prediction Analysis and Multigene Prognostic Signature Identification

The data-driven grouping (DDg) methods (one-dimensional (univariate), 1D-DDg, two-dimensional (bivariate) 2D-DDg) and statistically weighted voting grouping (SVWg) algorithms were used for patient's risk stratification onto two and three survival groups representing Kaplan-Meier survival functions (K-M functions) [37, 43-45]. These are well-established statistically based computational methods to identify optimized cut-off values of high-dimensional variable domains that transform large-scale variables to low-dimensional (discrete) scale-independent statistically weighted variables allowing for the selection of the most informative, robust, and reproducible categorical variables with the ability to stratify patient survival risk. In this study, an advanced version of the previously published software and algorithm [37, 43] was used. The following was a general description of how patient stratification in the risk groups can be utilized as a measure of survival prediction and as a method of selecting survival predictors (genes) for multivariate prognostic model.

### Example 4. 1D-Data Driven Grouping (1D-DDg)

A gene expression data set was assumed with i=1, 2, . . . , N genes whose intensities were measured for k=1, 2, . . . , K patients. The log-transformed intensities of gene i and patient k were denoted as y_{i,k}. Associated with each patient were a clinical outcome continuous data (e.g., survival time) and a nominal (yes/no) clinical event (e.g., tumor recurrence). Assuming that K clinical outcomes were negatively correlated with the vector of expression signal intensity yᵢ of gene i, patient k could be assigned to the high-risk or the low-risk group according to

$${x}_{k}^{i} = \{ \begin{matrix}1 ( high risk ) , & if {y}_{i , k} > {c}^{ℓ} , \\ 2 ( low risk ) , & if {y}_{i , k} < {c}^{i} ,\end{matrix} \qquad ( 1 )$$

where cⁱ denoted the predefined cutoff of the ith gene's intensity level. The clinical outcomes or events were subsequently fitted to the patients' groups by the Cox proportional hazard regression model [46]:

log hₖⁱ(tₖ|xₖⁱ,βᵢ)=αᵢ(tₖ)+βᵢ·xₖⁱ, (2)

where hₖⁱ was the hazard function and αᵢ(tₖ)=log h₀ⁱ(tₖ) represented the unspecified log-baseline hazard function; β was the 1×N regression parameters vector; and tₖ was the patients' survival time. To assess the ability of each gene to discriminate the patients into two distinct genetic classes (defined by Eq. (1)), the Wald statistic (W) [46] of the βᵢ coefficient of the model Eq. (2) was estimated by using the univariate Cox partial likelihood function [47], estimated for each gene i as

$$L ( {β}_{i} ) = {\prod }_{k = 1}^{K} {\{ \frac{exp ( {β}_{i}^{T} {x}_{k}^{i} )}{{\sum }_{j ∈ R ( {t}_{k} )} exp ( {β}_{i}^{T} {x}_{j}^{i} )} \}}^{{e}_{k}} , \qquad ( 3 )$$

where R(tₖ)={j: tⱼ≥tₖ} was the risk set at the time tₖ and eₖ was the clinical event at the time tₖ. The actual fitting of the model Eqs. (2-3) was conducted by the survival package in R (https://cran.r-project.org/web/packages/survival/index.html). The genes with the largest βᵢ Wald statistics were assumed to have better group discrimination ability and thus called survival significant genes. These genes were selected for further confirmatory analysis or inclusion in a prospective gene signature set. The log-rank statistics were also included in the algorithm and shown similar or in some cases slightly better p-value.

The stratification of patients in Eq. (1) depended on predefined cut-off values (cⁱ). In most real-world scenarios such values were not known in advance. The 1D-DDg method built on the described workflow, by identifying the ideal cut-off without needing any prior information. First, for each gene I, the tenth quantile (qⁱ₁₀) and the 90th quantile (qⁱ₉₀) of the distribution of K* signal intensity values were computed. For every value, the algorithm performed the splitting of patients (1), fitted the clinical event to the patient groups Eq. (2), and finally calculated the Wald statistic of βᵢ Eq. (3). In other words, within (qⁱ₁₀, qⁱ₉₀), the value* was searched which corresponded to the minimum βᵢ^{z} p-value (here z=1, . . . , Q) and that most successfully discriminated the two unknown risk groups.

It was noted that at the time of patient stratification it could not be told which group was associated with higher or lower risk. The 1D-DDg method predicted risk by analyzing the survival times of the groups. The group with lower mean survival times would be classified as “higher risk”, while the group with higher mean survival would be labeled as “lower risk”. According to this classification, two possible relationships existed between patient risk (lower risk, higher risk) and the expression pattern of a given gene (higher expressed, lower expressed). In the case of a parallel pattern, “higher risk—higher expression” or “low risk—low expression”, the relatively higher prognostic gene expression level was associated with the poorer prognosis (a gene exhibits pro-oncogenic behavior). In the case of anti-parallel pattern “higher risk—low expression” or “low risk—high expression”, the relatively higher prognostic gene expression level was associated with better prognosis (a gene exhibits tumor suppressor-like behavior).

The Rosetta and Metadata cohort datasets were used that contained both expression microarray data and the corresponding clinical information. The survival prediction analysis was focused on the identification of the shortlist of survival significant genes of the WAVE complex, RAC1 and NDEL1, all of which encoded proteins constituting or interacting with the WAVE complex. Input list of the genes included genes WASF (1,2,3), ABI (1/2/3), CYFIP (1,2), NCKAP1, BRK1, RAC1 and NDEL1, represented by the probe and probe sets localized in the 3′UTR of the selected genes on both microarray platforms.

The mRNA expression profiles of the selected genes were considered as putative predictors of the disease outcome. The 1D-DDg analyzed the survival prediction property of the Rosetta and Metadata expression microarray signals corresponding to WAVE complex members, and also RAC1 and NDEL1 as independent variables. An expression signal, called prognostic variable, was selected for further analysis if in both cohorts the DDg cut-off value(s) provided discrimination of the patients onto survival risk groups at p≤0.05. To keep a reasonable compromise between sample size, the imbalance of distinct risk groups in a patient cohort, reproducibility across the different cohort and prognostic significance of the putative prognostic variables selection step, it was also allowed to include in the prognostic variables set up to two variables, if for a given variable in one dataset (e.g., DFS, Metadata) p≤0.15. Thus, the output of 1D-DDg analysis for Metadata or Rosetta cohorts included the same list of reproducible prognostic variables (gene IDs) defined by the same gene lists, a similar survival prediction pattern of the identical variable (gene ID), cohort-specific gene expression cut-off values dichotomizing the patients on to relatively low-risk (code 1) and high-risk (code 2) groups [48, 43, 49, 45].

Using the results of 1D-DDg, the two-dimensional grouping (2D-DDg) method and statistically weighted voting grouping (SWVg), the robust and synergistic multi-gene prognostic signature was constructed. The ability of individual prognostic variables (voting weight) to stratify patients in risk groups was represented by the p-values associated with log-rank statistics.

### Example 5. Statistically Weighted Voting Grouping (SWVg)

SWVg is an automatic method of prognostic feature selection and disease risk prediction that allows the construction of an optimized, multivariable, prognostic classifier [48]. The input data was provided by the 1D-DDg method. The ability of individual prognostic variables to stratify patients was represented by the p-values associated with the Wald statistic (calculated in the 1D-DDg). These p-values were used to calculate the relative weight of individual variables in the multivariable classifier. This information was used to construct a decision rule and to assign a patient to one of the risk subgroups.

In practice, the list of genes was ordered in ascending order according to the p-values generated from 1-DDg. The weight wⱼ was calculated by the formula

$${w}_{j} = \frac{- log ( {p}_{j} )}{{\sum }_{m = 1}^{N} ( - log ( {p}_{m} ) )} , \qquad ( 4 )$$

where pⱼ was the p-value of gene in the 1D-DDg procedure. Then, the new numeric grouping value for sample i could be calculated by the formula

Gᵢ^{N}=Σⱼ₌₁^{N}wⱼGᵢⱼ, (5)

where N was the number of genes and Gᵢⱼ was the group allocation for sample i assigned by gene j in the 1D-DDg. In the case that samples were divided into two groups, patient i could be separated into two groups (2=“high-risk”, 1=“low-risk”) at a pre-defined cutoff value (G_{C}) of Gᵢ^{N} with the following:

$${y}_{i}^{N} = \{ \begin{matrix}1 ( high - risk ) , & if {G}_{1}^{N} > {G}_{C} \\ 0 ( {low}_{risk} ) , & if {G}_{i}^{n} \leq {G}_{C}\end{matrix} \qquad ( 6 )$$

A Cox proportional hazard regression model was estimated by using a univariate Cox partial likelihood function with the method described in the 1D-DDg procedure. Wald statistic of \hat{β}ⁱ was estimated and served as an indicator to evaluate the ability of group discrimination for gene j at cutoff G_{C}. The searching space of Gr was from 0.2 to 0.8, with an increment of 0.01 for each step. The G_{C} that provides the minimum log-rank p-values in the searching space was the optimized G_{C}. The above-described procedure was repeated for different N, which varied from 3 to the number of genes assigned. The number (Nₒₚₜ) and combination of genes were optimized for minimum log-rank p-values. A similar procedure was applied when the samples are divided into three groups. Two cutoff values (G_{C1}, G_{C2}, G_{C1}<G_{C2}) of F₁^{N} were selected and then used to calculate the grouping variable according to the following formula**:

$${y}_{i}^{N} = \{ \begin{matrix}1 ( l ow risk ) & if {G}_{i}^{N} > {G}_{C 2} \\ 2 ( intermediate risk ) & if {G}_{C 1} < {G}_{i}^{N} \leq {G}_{C 2} \\ 3 ( high risk ) & if {G}_{i}^{N} \leq {G}_{C 1}\end{matrix} \qquad ( 7 )$$

A Cox proportional hazard regression model and log-rank statistic estimates were computed. G_{C1} in Eq. (7) was searched in the range of 0.2 and 0.44, with an increment of 0.01 for each step; while G_{C2} was searched in the range 0.56 and 0.8, with an increment of 0.01 for each step. G_{C1}, G_{C2} were optimized for the minimum value of summation of pair-wise log-rank p-values of three survival curves.

The most significant and robust cut-off value did not always result in balanced groups (i.e., one group might only contain a few patients). Here the aim was to define risk groups that the smallest group contained at least 10% of the total patients. In cases where ‘the best’ cut-off value resulted in unbalanced groups, and other values could stratify patients with statistically significant Wald statistics, it was opted to use these alternatives.

Before the execution of Eq. (7), the group values of the high-risk group were recoded from 2 to 0. Using the modified values to calculate Gᵢ^{N} in Eq. (5), would result in Gⱼ^{N} closer to 0 for patients with higher risk. Conversely, patients with lower risk would have Gᵢ^{N} closer to 1. As a result, patients for whom Gᵢ^{N}>G_{C2} was true, would constitute the lower risk group. Patients with Gᵢ^{N} that was below G_{C1} would be in the high-risk group. Patients whose Gᵢ^{N} fell between G_{C1} and G_{C2} were classified as moderate risk.

To construct the multivariate prognostic signature, the SVWg started with paired gene expression data using the two-dimensional grouping (2D-DDg) method [48, 49, 45]. For the given two variables domain and the 1D-DDg determined cut-off values of these variables, the 2D-DDg identified two mutually excluded sub-domains in the 2D domain that maximized discrimination of all subjects (patients) onto low- and high-risk groups. The possible distinct sub-domain combinations in the 2D domain were called ‘designs/models’ of the patient's grouping.

SWVg added the next prognostic variable that increased differentiation between risks of the groups and allowed a selection of a synergistic multi-variable signature based on the summation of the statistically weighting variables in a stepwise multivariable fashion. Less stringent statistical criteria (weights) were used by SWVg when the next most significant prognostic variable was added to the survival prediction model. The sample size and data quality constraints were included in the algorithm allowing the SWVg to minimize the number of prognostic variables (predictors) and to reduce the signature identification overfitting risk keeping high confidence and reproducible prognostic multivariate model.

The multivariate method started with the most significant prognostic variable (1-st rank predictor) paired with the next most significant predictor. These features in combination provided a synergistic effect, robust prognostic signature, and provided consistency between the signatures derived in the cohorts.

### Example 6. Optimization of the 2D-DDg Method for Correlated Covariates (Gene Expression Value Pairs)

In many datasets, the gene pairs (A and B) expressions might be correlated positively or negatively due to some context regulatory mechanisms (interaction due to common medical condition(s) and similar treatment). The paired correlation analysis could be used to improve the significance and robustness of patient's risk group stratification. In this section, an extension of the 2D-DDg method was described.

It was assumed that N denoted the number of non-duplicated samples of the population (patient cohort). It was assumed that {X, Y} denoted the N random variable (r.v.) pairs (e.g., gene expression levels in the N samples that associated with N patient survival data (event and time after disease diagnostics or last follow up), where the expression levels X and Y of the genes A and B respectively. If the correlation measure between r.v. X and Y significant, the DDg defined risk group separation cut-off value (gene expression value determined a patient to the given risk groups) of bivariate r.v., could be optimized due to the variable's dependence. In such cases, the ‘interaction effect’ (synergy) between A and B data into 2D-DDg prediction analysis can be defined as follows.

The method calculated the Kendal tau (or Spearman) correlation coefficient between all possible paired of r.v., specified significantly correlated pairs, and then parametrized the linear regression model quantifying the stochastic association between two r.v.

Y=α+βX+ε (8)

where x was the vector of gene A expression values, x={x₁, x₂, . . . , x_{N}}; y was the vector of gene B expression values, y={y₁, y₂, . . . , y_{N}}; ε represented an additive error term that might stand un-modeled determinants or random statistical noise: ε={ε₁, ε₂, . . . ε_{N}}. N was the number of samples; α and β were parameters of the linear regression model. α was a y-intersect of the line and β was a slope of the line. The parameters were estimated using the least squares method. The estimated parameter values denoted as â and \hat{β}.

Using parametrized Eq. 8 for the vector component pair (x,y−\hat{α}) defined in the form

(yᵢ−\hat{α})=\hat{β}ₓ_{ᵢ}, i=1,2, . . . ,N, (9)

the shortest distance of a particular point Q (x, y) from the regression line was calculated. To do this a rotation of orthogonal coordinate system formula of point Q {x,y−\hat{α}} was used as the following

x̅ᵢ=xᵢ cos γ+(yᵢ−\hat{α})sin γ; (10)

y̅ᵢ=xᵢ sin γ+(yᵢ−\hat{α})cos γ; (11)

where {x̅ᵢ,y̅ᵢ} were the coordinates of point Q in the new orthogonal coordinate system rotated on the angle γ. Using trigonometric formula, \hat{β}=tan γ, and it was obtained

x̅ᵢ=(xᵢ+\hat{β}(yᵢ−\hat{α}))/(1+\hat{β}²)^{1/2}, (12)

y̅ᵢ=(−\hat{β}xₜ+(yₜ−\hat{α}))/(1+\hat{β}²)^{1/2}. (13)

Eqs. (12)-(13) were used in the study for the calculation of new coordinates of the objects and corrected cut-off values C{x̅*,y̅*} defined by DDg for prediction of the low- and high-risk groups in the patient cohort.

The rotation of orthogonal coordinate system approach was included and used in the 2D-DDg method to improve the significance of the patient's separation on the relatively low- and high-risk groups. The analysis showed that in the high-correlated genes, this method improved the statistical significance of results obtained in DDg methods, but also could lead to more robust grouping and reproducibility of the risk model across distinct patient cohorts. For instance, in the case of ABI1-BRIK1 pair of the Rosetta cohort, the standard 2D-DDg survival prediction analysis of DMFS provided near the borderline statistical significance of patients grouping (p<0.05). However, a strong positive correlation between expressions of these two genes was found (p<0.0001), suggesting common co-regulatory mechanisms.

### Example 7. Prognostic Models, Correlations, and Reproducibility of the ABI1-Based Prognostic Signature Genes

According to the selection criteria of prognostic variables (Methods), 1D-DDg selected 5 genes of WAVE complex (ABI1, BRK1, CYFIP1, CYFIP2, and WAVE3) and 2 genes (RAC1 and NDEL1) encoding the proteins RAC1 and NADEL which exhibited ‘interaction’ with WAVE complex components. The 7 genes were representative be unique probe sets on Affymetrix U133 A&B and Rosetta microarray platforms (Table 2). FIGS. 9 and 10 and Table 3 showed that across different microarray platforms DFS and DMFS survival patterns ABI1, BRK1, CYFIP1 and RAC1 were commonly reproducible and classified as pro-oncogenic, while CYFIP2, NDEL1 and WAVE3 were mostly classified as tumor suppressor-like genes. However, because the system of interactive molecules was open, stochastic, and non-linear for some genes (e.g., WAV3) the variations of these prognostic properties (as a component of the system) could be unstable and expressed alternative functions.

It was noted that over data sets and event types (e.g., DFS, DMFS) the prognostic pattern of expression changes for some individual genes (e.g., WASF3) was in some cases not the same (classified as proto-oncogene or tumor suppressor like). However, the pro-oncogenic pattern (upregulated expression—poor prognosis) of ABI1, BRK1 and RAC1 or the ‘tumor suppressor’ pattern (upregulated expression—good prognosis) of NDEL1 and CYFIP2 expression was highly reproducible between the datasets.

In the context of co-expression, the METABRIC, Rosetta and Metadata data, ABI1 expression was positively correlated with the expression of BRC1, CYFP1, NDEL1. It was also not correlated with RAC1 expression and was negatively correlated with CYFP2 expression.

### Example 8. Materials and Methods

Mouse Primary Tumors RNA-seq: Gene expression profiles from primary breast tumors of PyVT heterozygous and homozygous mice with and with Abi1 disruption were detected with the Illumina NextSeq platform (GSE162815). Two tumors from a single mouse from each of the four groups were sequenced. Two runs were performed on consecutive days for increased depth. Illumina's breast cancerl2fastq program was used for the conversion of base calls to FASTQ files. This resulted in two read files due to the paired-end sequencing protocol. STAR was used to align sequences to the GRCm38/mm10 mouse genome. STAR was also used for the quantification of reads per gene. Raw counts between tumors and days for each genotyped were summed for maximum depth. Fold change was calculated between Abi1 wild-type and Abi1 knockout mice for each genotype.

Animals: Transgenic PyMT mice (JAX no. 022974; C57BL6) and mammary-specific Cre mice (JAX no. 003553, Line D; mixed strain) were purchased from Jackson Laboratory. Abi1-floxed mice were generated by the Kotula Laboratory [16] (MGI:4950557; Abi1^{tm1.1Lko}, C57BL6). Female PyMT mice with conditional Abi1 knockout were generated by crossing PyMT transgenic males to homozygous Abi1 females to produce PyMT transgenic males heterozygous for Abi1 floxed allele (PyMT;Abi1 fl/wt). PyMT;Abi1(fl/wt) males were backcrossed to homozygous Abi1 females to produce PyMT;Abi1(fl/fl) males. In parallel, transgenic Cre animals were crossed with homozygous Abi1 animals to generate transgenic Cre animals heterozygous for Abi1 [MMTV-Cre;Abi1(fl/wt)]. To generate experimental animals, male PyMT;Abi1(fl/fl) were crossed to female MMTV-Cre;Abi1(fl/wt). All breeders used were at least 8 weeks of age. Genotyping was performed using ear snips (Transnetyx, Cordova, TN). As mammary glands were the tissue of interest, only female experimental animals were analyzed. Female animals were sacrificed at designated timepoints (5, 7, and 12 weeks, for developmental studies, n=5 animals per genotype; or seven weekly time points starting with the tumor detection (at week 0, 1, 2, 3, 4, 5, and 6, n≥6 mice), when tumors reached 2.0 cm, or when animals displayed signs of distress as per the guidelines of the National Research Council Committee on Recognition and Alleviation of Distress in Laboratory Animals. For primary and lung metastasis tumor studies animals (n≥6 mice) were sacrificed age between 17-26 weeks. All animals used in the studies described herein were housed in ventilated microisolator caging under HEPA-controlled environmental conditions and maintained under the supervision of the SUNY UMU Institutional Animal Care and Use Committee (IACUC no. 393).

Tumor Palpation and Measurements: Starting at weaning age, all female PyMT animals were palpated and measured for tumors bi-weekly. Tumor measurements and volume calculations were performed as previously described [50]. Total tumor burden over time was calculated for each animal (n=6/genotype) and was plotted against the time since primary breast tumor was detected by palpation.

Mathematical Models and Estimated Parameters in Analyses of Primary Tumor Kinetics and Pulmonary Metastatic Node's Size Frequency Distribution: The parameters of the tumor volume kinetics were estimated using the exponential function

ƒ(t;a,b)=(a−b)*exp(ax),

where t was time, parameter a was the rate of cell volume growth and (a−b) was the initial tumor volume. SigmaPlot-13 software was used to perform nonlinear regression analysis and the results visualization.

Proliferative Activity and Histological Parameters of the Mouse Primary Tumors: Mouse mammary parenchyma less than (<20 wks) or greater than 20 weeks (>20 wks) of age were examined by a blinded pathologist. Histologic sections of healthy control, homozygous control, heterozygous control, homozygous Abi1(KO, −/−), and heterozygous Abi1(KO,−/+) breast parenchyma were compared. The murine grades were determined according to published histologic criteria [35]. The Ki-67 index was expressed as percent positivity from 500 nuclei counted in areas of highest positivity. The comparative analysis was performed for each group of mice vs normal and corresponding negative contours (breast parenchyma) of heterozygous Abi1 (KO,−/+) and homozygous Abi1 (KO,−/−) samples. Unpaired non-parametric Mann-Whitney U test was performed at p<0.05.

Lung Metastasis Quantification: To quantify the metastatic area throughout the lung tissue, three 5-μm sections from formalin-fixed paraffin-embedded mouse lungs (sectioned every 50 μm) were collected from each group (n≥6 animals per genotype), stained with hematoxylin and eosin and imaged using an Omnyx digital pathology scanner (GE Healthcare) [51]. Images were quantified for the total number of metastatic foci using ImageJ software (NIH) and subjected to statistical analyses.

Western Blot Analyses: Western blots were performed as previously described [16]. Blots were probed with the following primary antibodies: ABI1 (Rockland; 1:1000), ABI2 (P-20, Santa Cruz Biotechnology; 1:500), ABI3 (GeneTex; 1:1000), WAVE1 (K91/36, Millipore; 1:1000), WAVE2 (H-110, Santa Cruz Biotechnology; 1:1000), WAVE3 (W4642, Sigma Aldrich; 1:1000), or β-actin (AC-15, Sigma Aldrich; 1:10,000). Blots were incubated with SuperSignal West Pico or Femto ECL reagents (Thermo Fisher) and imaged using a PxiTouch imaging system (SynGene).

Immunohistochemistry, Histology, and Whole Mount Analysis: Immunohistochemical staining was performed with antigen retrieval following standard protocols. Tissue sections of normal mammary tissue were stained with anti-CK8 (TROMA-I, DSHB, Iowa, 1.1000) and anti-CK14 (PRB-155P, Covance, 1.250). Tumor sections (≥3 animals/genotype) were stained with the following antibodies: ABI2 (P-20, Santa Cruz Biotechnology, 1.250), WAVE1 (K91/36, Millipore, 1.250), WAVE2 (H-110, Santa Cruz Biotechnology, 1.250), and WAVE3 (Abreast canceram ab110739, 1.100). Stained sections were mounted on coverslips using Cytoseal XYL (Fisher) and imaged using a Nikon Eclipse Ci-L upright microscope. Formalin-fixed tumor specimens were stained with hematoxylin and eosin for histopathologic review. Grading of murine tumors were performed according to Fluck and Schaffhausen's review of the model pathology [35]. Briefly, tumors were assigned a score of 0 (normal breast parenchyma), 1 (mammary hyperplasia consisting of dense lobules), 2 (mammary intraepithelial neoplasia; the murine correlate of ductal carcinoma in situ), 3 (early carcinoma characterized by early stromal invasion), or 4 (late carcinoma). The mitotic rate was determined by counting the number of mitotic cells in 10 high-power fields (hpf). The mitotic rate was calculated for the areas of the tumor with the highest grade. Tumor sections were also stained with Ki-67, with nuclei in cells in the highest-grade areas counted to determine expression, which was reported as the percentage of positivity.

For whole mount staining, mammary glands were processed as previously described [52]. Stained whole-mounted tissues were imaged using a Nikon D610 camera, and images were subjected to morphometry using ImageJ software (NIH). Terminal end buds, ductal length, and ductal branching were quantified as previously described [53, 54].

Statistics: Each cellular or biochemical experiment had technical (n≥3) and biological (n≥3) repeats. To determine statistically significant differences involving more than 2 biological groups, 1-way and 2-way ANOVA were used followed by t-test, non-parametric tests, generalized univariate and multivariate linear models, correlations other analyses as stated elsewhere in the manuscript using Statistica 13, StatSoft); p-value less than 0.05 was considered significant. Categorical data analyses were carried out using Sytel Studio-9 software (Sytel Inc. Pume). Kinetic analysis and non-linear model's parameterization were done using SigmaPlot-13 (Systat Software) software.

Ethics Approval and Consent to Participate: All animal studies were performed according to guidelines approved by the Institutional Animal Care and Use Committee of SUNY Upstate Medical University (Protocol no. 393). Publicly available datasets were used for all patient-associated bioinformatics analyses in this manuscript.

### Results

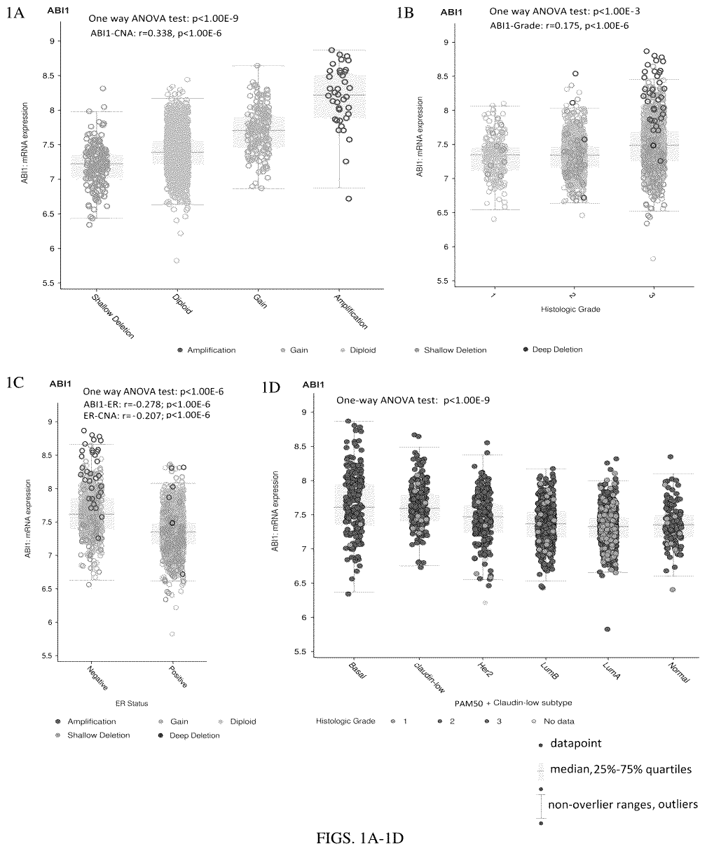

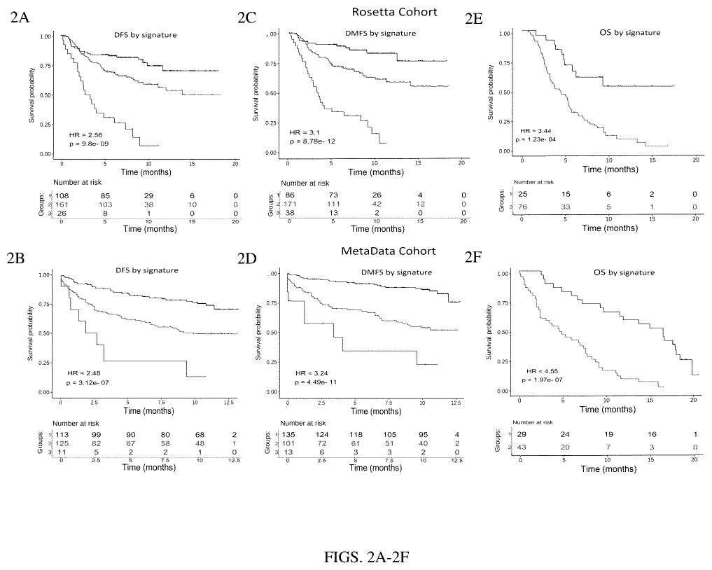

Upregulation of ABI1 Gene Expression in Primary Breast Cancers Correlates with Aggressive, Basal-Like Phenotype and Metastatic Predisposition. ABI1 is an essential part of the WAVE regulatory complex, a major promoter of actin filament nucleation is often exploited by invasive tumor cells [55]. To elucidate the significance of the ABI1 in the pathobiology of human breast cancer, a retrospective analysis of METABRIC data of 1904 breast cancer patients were carried out (FIG. 1). It was found that expression of ABI1 in primary tumors is strongly associated with copy number alterations (CNA) (FIG. 1A), overexpression with histologic grade 3 (FIG. 1B). There was a significant negative correlation ABI1 mRNA as well as ABI1 CNA with ER (+) status (FIG. 1C) and no correlation with the lymph node (LN) status of the patients (FIG. 11).

Moreover, ABI1 overexpression is associated with highly aggressive (grade 3) basal-like and claudin-low breast cancer subtypes (FIG. 1D). Additionally, using cBioPortal for Cancer Genomics tools, it was observed that high-expressed and gained & amplified CNA ABI1 are significantly enriched in the high genome instability integrative cluster 10 [42]. The cluster 10 molecular subtype is enriched by basal-like cancer subtype tumors and clinically defined as triple-negative, highly aggressive, drug resistance and high-risk metastasis tumor genes, that includes numerous signaling molecules, transcription factors, mitotic and other cell division genes associated in trans with this deletion event in the basal cancers, including alterations in AURKB, BCL2, BUB1, FOXM1, KIF2C, KIFC1, RAD51AP1, TTK and UBE2C. Notably, many of these molecules are included genetic grade and poor survival outcome signatures [56, 42, 40, 57]. For instance, TTK (MPS1), a dual-specificity kinase that assists AURKB in chromosome alignment during mitosis and promotes aneuploidy in breast cancer [42].

Thus, ABI1 expression shows strong positive correlates with histologic grading, negative correlation with ER status, and represents correctly the known ranked-order of breast cancer subtypes according to their genetic grading classification (FIGS. 1A-D; [56, 42, 40, 57], 54]. These findings allow to consider ABI1 transcription level as a functional score of indicating i) this gene locus instability, ii) ER(−) status of the primary tumor, iii) histologic grading system estimator and iv) a genetic variable that represents correctly known ranked-order of breast cancer subtypes that reflect genetic grading and drug sensitivity/resistance of the tumor subtypes/groups.

Additionally, multivariate testing ABA1 expression variation as a random function of CNA, ER status and tumor subtypes showed that CNA in basal-like tumor subtype samples provides a major explanatory contribution of ABI1 expression variation in our data (p<1.00E-6; Two-way ANOVA, Statistica 13).

Survival Prediction Analysis Identifies ABI1 as Breast Cancer Metastasis Prognostic Marker and an Important Component of the Multigene Metastasis Prognostic Signature. Associations of survival data were analyzed with microarray gene expression profiles of well-established publicly available breast cancer datasets [40, 41, 39]. These datasets were used to construct the Metadata and Rosetta microarray datasets.

It was focused on the identification of the role of ABI1 expression in breast cancer survival associated with cancer progression/recurrence (defined DFS time) and metastatic process (DMFS time). Table 2 provides ABI1 annotation and unique probe sets on Affymetrix U133 A&B and Rosetta microarray platforms utilized in the analysis. For stratification of the patients onto risk groups, 1D-DDg was utilized, which approximates patient risks by analyzing the survival time functions of two (or more) patient groups given by the prognostic variable cut-off value(s) estimated statistically in a given patient cohort. The examples of implementation of 1D-DDg results for Rosetta and Metadata cohorts are presented in FIGS. 9 and 10. Each figure shows the gene panels of two K-M plots of disease-free survival (DFS) (FIG. 9) and distant metastasis-free survival (DMFS) (FIG. 10), respectively. The groups of the patients assigned to relatively low risk (step function line indicated by black color) and high risk (step function line indicated by red color) K-M survival functions are defined by the gene expression cut-off value calculated by 1D-DDg. The group with higher mean survival time is labeled as ‘low risk’, while the group with lower mean survival time is labeled as ‘high risk’. According to this classification, two possible relationships exist for the patients with lower and higher risks and the expression pattern of a given gene (higher expressed, lower expressed). In the case of a parallel pattern, “higher risk—the higher the expression” or “low risk—the lower the expression”, the relatively higher prognostic gene expression level is associated with the poorer prognosis (a gene exhibits pro-oncogenic behavior). In the case of anti-parallel pattern “higher risk—the lower the expression” or “lower risk—the higher the expression”, the relatively higher prognostic gene expression level is associated with better prognosis (a gene exhibits tumor-suppressive-like behavior).

Importantly, the prognostic association “higher risk—the higher the expression” of ABI1 was statistically significant and reproducible over breast cancer cohorts (FIGS. 9-10). These results consist of the 1D-DDg analysis of the gene expression of the ABI1 gene and ABI1 protein in breast cancer patients found in RNA-seq and proteomics databases (FIG. 12).

The high-risk group of patients in the cohorts is proposed to be associated with a higher frequency of metastatic events. The metastasis event enrichment analysis (Table 4) showed that in both cohorts the higher risk group was significantly enriched by metastatic events vs. the lower-risk group. The fold change (FC) enrichment of metastatic event and p-value was calculated using the exact test of two binomial distributions, that showed FC=1.38, p=0.05 in Rosetta and FC=1.96, p=0.033 in Metadata dataset, respectively.

Next, the results generated by the 1D-DDg survival prediction method, which automatically selects survival significant prognostic variables (survival significant genes represented by microarray probes), were used as the input data for the 2D-DDg [29,32] that identifies the interaction effect between paired prognostic variables (gene pairs) [29,32]. FIG. 13 shows the result of the implementation of 2D-DDg survival prediction to Rosetta data (DFS and DMFS, respectively). These results show that in most gene pairs ABI1 improves the balance between risk groups and in some cases the bi-variate partition of the patients provides more confident risk group differentiation. Similar results were observed for the Metadata data set (not shown).

In both the cohorts, ABI1 expression is positively correlated with the expression of BRK1, CYFP1 and NDEL1, but is not significantly correlated with the expression of CYFP2, WASF3, and RAC1 (p<0.05, Spearman). These findings in most cases consist of the correlation analysis of ABI1 and other genes expression from the METABRIC datasets (Table 5). It is noted that WAVE3, CYFIP1 prognostic models may be more data variation- and noise-sensitive.

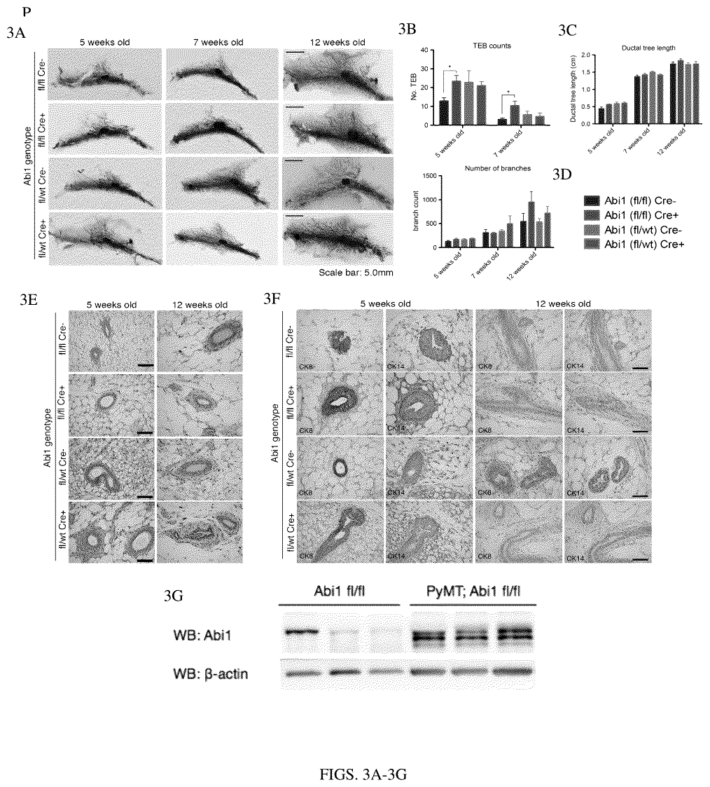

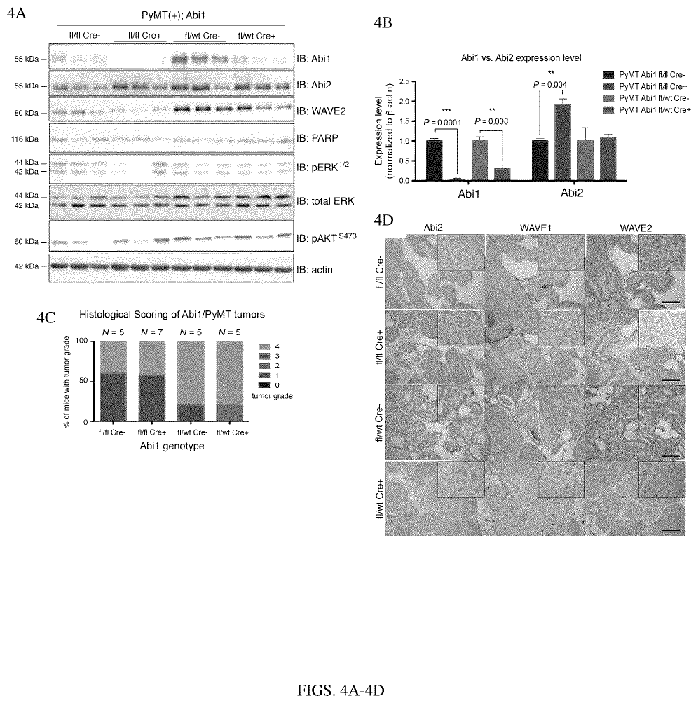

The SWVg algorithm was used to construct a survival group prediction model based on the combinations of 1DDg-defined gene expression level models. FIGS. 2A-D shows that in both Rosetta and Metadata cohorts the method revealed a high confidence stratification of the patients onto three risk groups with high-, intermediate- and low-metastasis-free survival time, called the ABI1-based 7-gene prognostic signature. Similar results were obtained for DFS time (results are not shown). Overall, the genes of the ABI1-based 7-gene prognostic signature provide robust functional associations and high-confidence survival prediction properties.

ABI1-Based Prognostic Signature as a Predictive Tool for a Metastatic Event of Breast Cancers. Importantly, the ABI1-based prognostic signature could serve as a predictive tool for a metastatic event of breast cancer patients. Table 6 shows that in the case of DMFS the high- and intermediate-risk groups are highly enriched for patients with metastatic breast cancer events compared to the low-risk group (62% and 44% vs. 15% for Metadata and 79% and 34% vs 15% for Rosetta cohorts). The median and mean time values of metastatic events showed an inverse order in these risk groups. These findings suggest that the signature values defined in primary breast cancer samples can be used for the quantitative prediction of distant metastasis events and the time interval of metastatic event occurrence.

Using the ABI1-based prognostic signature genes and specifying their expression cut-off values as done before, the patients can be further stratified with metastatic events into relatively lower and higher OS time risk groups (FIGS. 2E-F). These results suggest that the ABI1 and other genes of the prognostic signature are involved in the progression towards metastatic disease and may be mechanistic regulators of a subset of metastatic breast cancers.

To compare the prognostic significance, Rosetta and Metadata cohort's clinical and gene expression data were used and were compared the ABI1-based 7-gene prognostic signature with commonly used clinical markers: estrogen receptor (ESR) and lymph node (LN) status (FIG. 11). While ESR status shows significant differences in DMFS in the survival of the Rosetta cohort (low vs. high expression) (FIG. 11A), it was not a predictive factor in the Metadata cohort (FIG. 11C). An opposite prognostic pattern was observed for LN status: it is not significant in the Rosetta cohort (FIG. 11B) but shows prognostic significance in the Metadata cohort (FIG. 11D). Additionally, univariate and multivariate analyses showed that LN and ER status is insufficient for reliable prediction of 3 risk groups (not shown).

Overall, the ABI1-based prognostic signature provided robust, reproducible and high confidence prediction models of DFS, DMFS and OS (FIG. 1-2, FIG. 9-10, Tables 3, 6) and demonstrates high performance across different cohorts (FIG. 2, Table 3). Reproducibility of risk stratification of the patients with metastases in the Rosetta and Metadata datasets based on OS time supports this statement. Furthermore, the Abi1-based signature predicts distant metastatic events more accurately than commonly used clinical factors (Table 7).

Loss of Abi1 Does not Grossly Affect the Long-Term Development of Normal Mammary Glands. While implicated in breast tumor progression, the role of ABI1 in normal mammary tissue remains unknown. To ensure that phenotypes that may be observed in the Abi1 knockout (KO) breast tumor model result from the effects of ABI1 protein loss on tumor progression and not from an otherwise global effect on breast tissue, Abi1 was conditionally deleted from mammary epithelial cells of non-tumor-bearing animals. As with most mammals, mouse mammary gland development occurs postnatally [58]. Mice are born with rudimentary mammary fat pads that develop into functional mammary glands upon the onset of puberty. Beginning at five weeks of age, the ductal tree begins to penetrate the mammary fat pad and continues until sexual maturation. This dynamic tissue reconstruction allows for examination of classical mammary structures such as ductal branches and terminal end buds (TEBs) (for an extensive review of mammary gland development [58]). Expression of the mammary-specific CRE recombinase is under the control of the murine mammary tumor virus (MMTV) promoter and begins at ˜21 days, allowing to observe phenotypic changes in normal mammary gland tissue upon ABI1 loss [59].

To determine the effects of ABI1 loss on the development and structural integrity of normal mammary tissue, a whole mount analysis was performed on the inguinal mammary gland. Gross examination revealed a modest impact of ABI1 loss on mammary gland development (FIG. 3A). Changes in the total number of terminal end buds (TEBs) as well as the number of ductal branches and total ductal tree length were examined. TEBs are highly proliferative, tear-shaped structures found at the distal end of the ductal tree that penetrate the mammary fat pad to facilitate ductal tree elongation and are involute upon completion of ductal tree extension [58]. Morphometry of mammary gland whole mounts showed a significant increase in the number of TEBs upon homozygous ablation of the Abi1 gene and a trend towards increased branching, the latter of which did not reach significance (FIGS. 3A and C); however, this does not seem to impact long-term gland development, as ductal tree elongation remained unaffected (FIG. 3D). Heterozygous Abi1 KO glands showed sustained TEB counts in 5- and 7-week-old whole mounts (FIG. 3B-D).

In addition to dynamic tissue reorganization, mammary glands also have classically defined ductal structures. Murine mammary ducts are defined as lumens lined by an inner layer of luminal epithelial cells and an outer layer of myoepithelial cells [58]. Thus, analysis of this cellular organization would indicate whether there are organizational defects within the mammary duct upon Abi1 deletion. Gross pathological examination of hematoxylin and eosin (H&E)-stained mammary gland sections show the unaltered organization of epithelial cells and connective tissue within ducts (FIG. 3E). Immunohistochemical staining for cytokeratins 8 and 14, which mark myoepithelial and luminal epithelial cells, respectively, shows similar staining patterns in control and Abi1-null mice (FIG. 3F) [60]. Taken together, it is shown that ABI1 loss does not affect the long-term mammary gland development of healthy mice.

ABI1 Protein Level and Gene Dose Regulate Tumor Growth in PyMT Animals. ABI1 overexpression has been implicated in promoting an aggressive breast cancer phenotype; however, its exact role in mammary tumor progression is still unclear [33, 34, 32]. First, it is established that PyMT transgene induces expression of Abi1 in primary tumors vs. normal mammary gland epithelium of Abi1 floxed mice (FIG. 3G), therefore it is concluded that PyMT mouse recapitulates overexpression of ABI1 observed in human tissue, and thus it is an appropriate model to examine the role of ABI1 in breast cancer tumor progression. To determine efficiency of Abi1 gene loss in the Abi1 KO PyMT animals, deep RNA-seq analysis of representative primary tumors of each genotype was performed (Table 1). It was found that Abi1 gene expression follows gene dosage effect as expected: 15.4-fold in homozygotes and 2-fold in heterozygotes vs. their respective controls. Several members of the WAVE complex were modestly downregulated or retained their expression in Abi1 KO tumors vs. controls, while Wave3 (Wasf3) was upregulated and Cyfip2 was downregulated. An opposite effect on several WAVE complex genes expression in the heterozygous vs. homozygous animals was apparent (Table 1).

Interestingly, comparative analysis of the basal-like vs. luminal breast cancer cell type markers in ABI1 KO mice showed that the genes of basal-like cells (Krt14, Vim) are responded to Abi1 depletion, however luminal cell type genes markers (Krt8, Krt18, Sox9, Estr1) do not (Table 8). The directionality of gene expression of Krt14 and Vim in heterozygous and homozygous mice was different.

It is shown that Abi1 KO mouse embryonic fibroblasts reliably show downregulation of WAVE2 [16]. Consistent with this finding, Western blot analysis of Abi1 KO breast tumors (tumor lysates from 3 mice/genotype) showed an appreciable reduction in WAVE2 expression in the absence of ABI1, recapitulating previously observed WAVE complex dynamics and dependence of complex stability on Abi1 gene status (FIG. 4A) [16, 17]. Interestingly, WAVE2 expression remains relatively stable in heterozygous Abi1 KO tumors, suggesting that a single copy of Abi1 is enough to sustain WAVE complex stability to some degree, noting that there is still a noticeable loss in WAVE2 expression. Also, densitometric analysis of the Western blots revealed significant upregulation of ABI2, another member of the ABI family, only in homozygous Abi1 KO animals, in agreement with the previous findings (FIG. 4B) [16].

Based on the Western blot findings, next it was examined whether altered WAVE complex expression in the absence of ABI1 was recapitulated by immunohistochemical staining of tumor tissue (FIG. 4D). Similar to the Western blot results, Abi1-null tissue shows increased ABI2 expression in the cytosol while WAVE2 shows moderate downregulation overall. WAVE1 is modestly expressed regardless of ABI1 status; therefore, it may not play a role in breast tumorigenesis in this model. Due to their ubiquitous expression, ABI1-WAVE2 complexes are considered canonical WAVE complexes that drive F-actin polymerization during cell processes [61]. As there is a concomitant loss of WAVE2 upon Abi1 KO but sustained tumor growth in PyMT mammary tumors, it is possible that other factors contribute to ARP2/3-mediated actin polymerization. Moreover, overall primary mammary tumor histopathology was not affected upon ABI1 loss (FIG. 4C, Table 9). While most of the primary tumors in either control or homozygous Abi1 knockout animals remain in grades 3 or 4, some tumors in the heterozygous Abi1 knockout appear to be in grade 2, further highlighting the impact of single copy Abi1 deletion as opposed to homozygous deletion and suggesting other mechanisms may be induced in the complete genetic absence of Abi1.

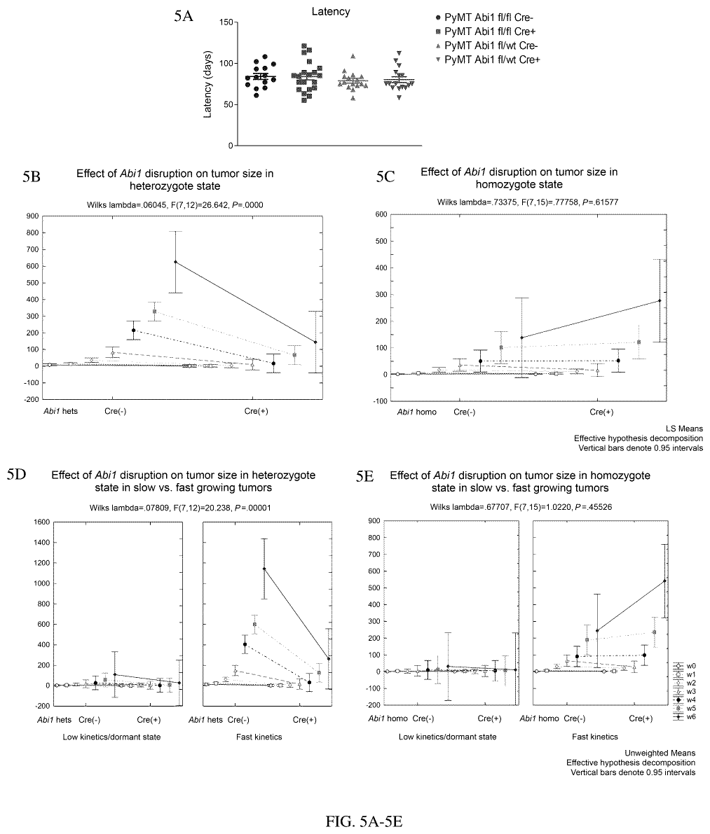

ABI1 Gene Dose Regulates Primary PyMT Tumors Growth Kinetics. To determine the impact of Abi1 disruption on mammary tumor initiation and progression, the Abi1 KO mouse model was used to study the impact of Abi1 loss on tumor progression and characteristics in the PyMT-driven breast cancer. The PyMT model initiates spontaneous tumor formation with most mammary glands developing tumor nodes. Interestingly, KO mice Abi1 does not significantly impact primary tumor latency (FIG. 5A). To determine the effects of Abi1 expression on breast cancer progression, heterozygous and homozygous KO mice were used to study the growth kinetics (i.e., tumor volume changes over time) of sporadically occurring tumors. Tumor size was measured bi-weekly, starting from first day of tumor palpation. Datasets from Abi1 homozygous Cre(+) (n=11), Abi1 heterozygous Cre(+) (n=11), Abi1 homozygous Cre(−) (n=13) and Abi1 heterozygous Cre(−) (n=13) samples were collected and analyzed (FIGS. 5B-E). The tumor kinetics showed two growth patterns. The analysis of tumor volume kinetic data in mice identified two tumor growth patterns, which are growing with very low or fast rates across all four genotypes (FIGS. 5B-E, Table 10). The fraction of tumors exhibiting the slower growth varied between 54% and 64% across the four experimental groups. The breast tumor samples showed stable exponential growth with either moderate or high growth rates (FIGS. 5B-E, Table 10). Using the one-way ANOVA test, it was found that in Abi1 heterozygous KO model samples the tumor growth kinetics was strongly suppressed vs. control (FIG. 5D), while no significant effect was found in Abi1 homozygous KO model tumor samples with some positive trend in the opposite direction in faster-growing tumors (FIG. 5E).

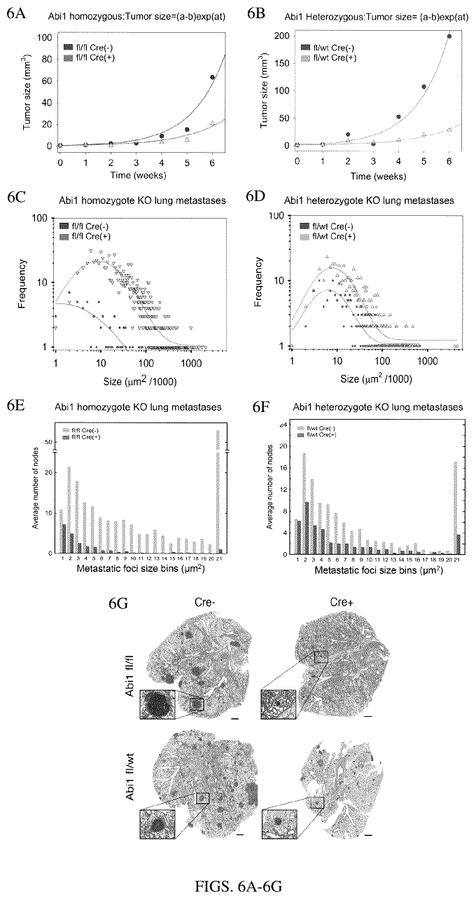

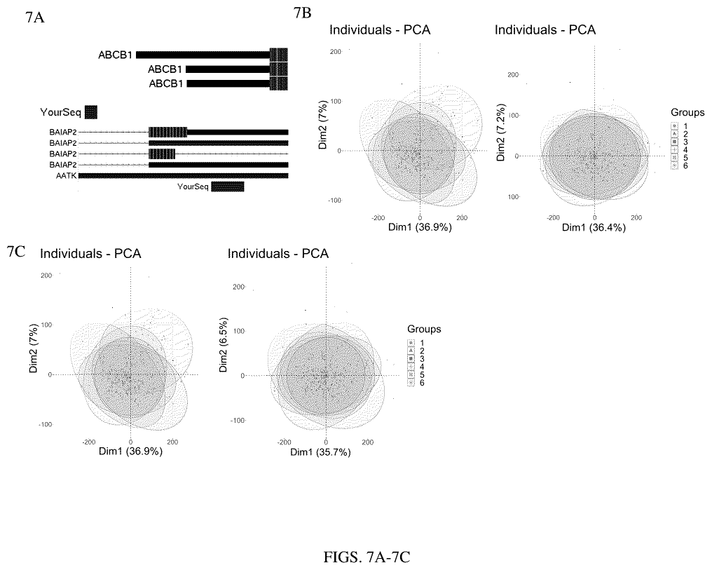

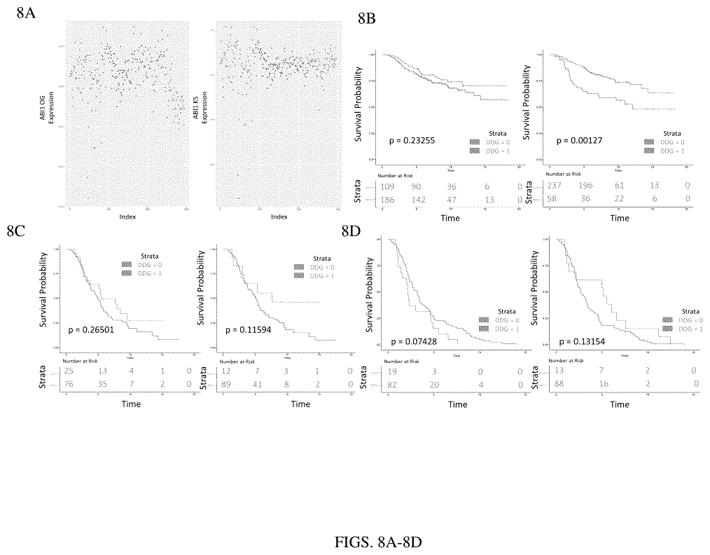

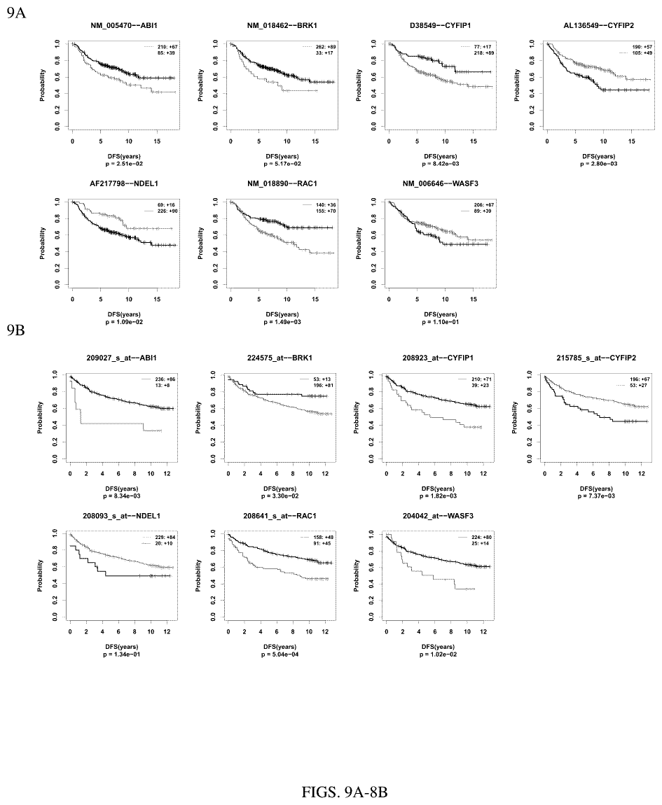

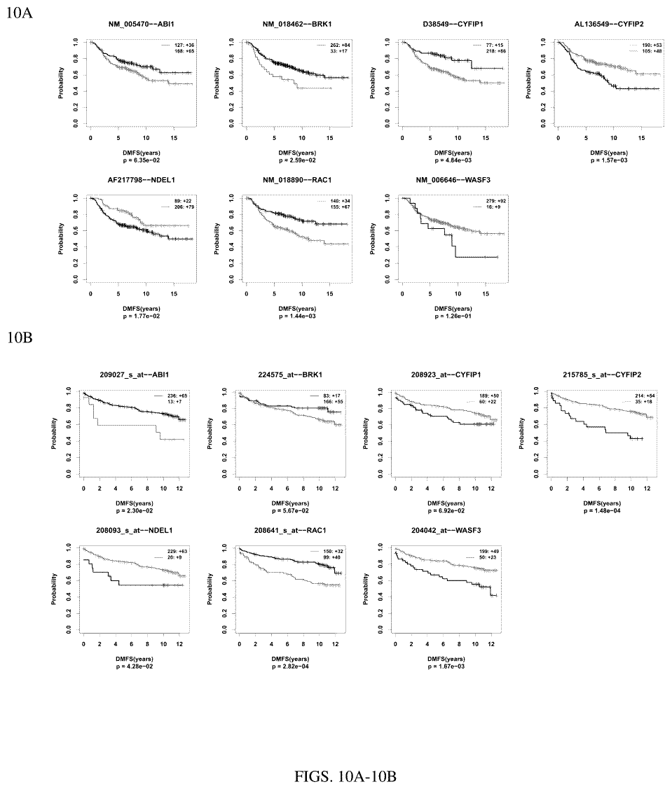

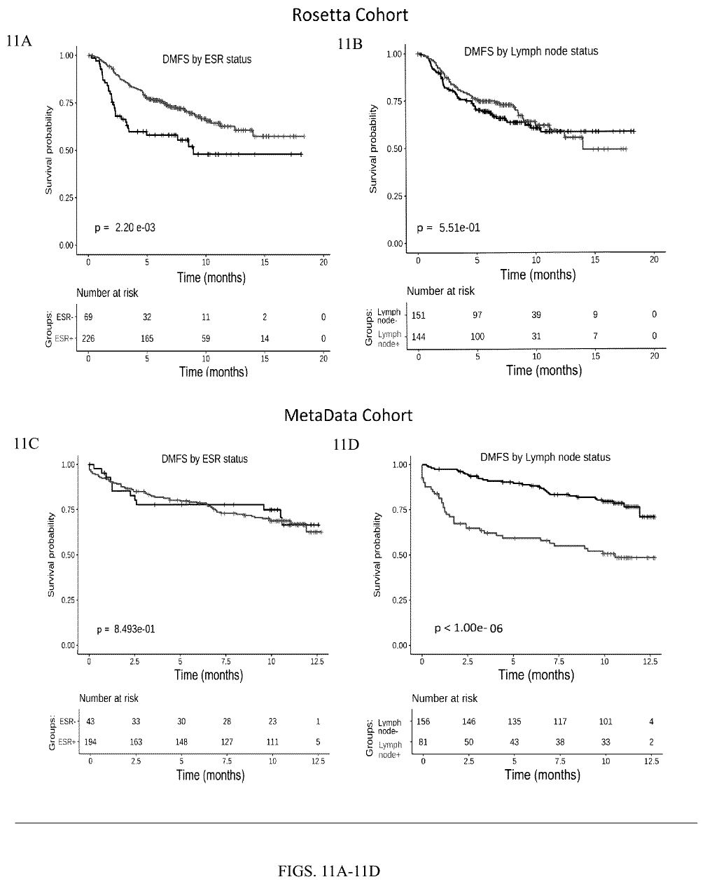

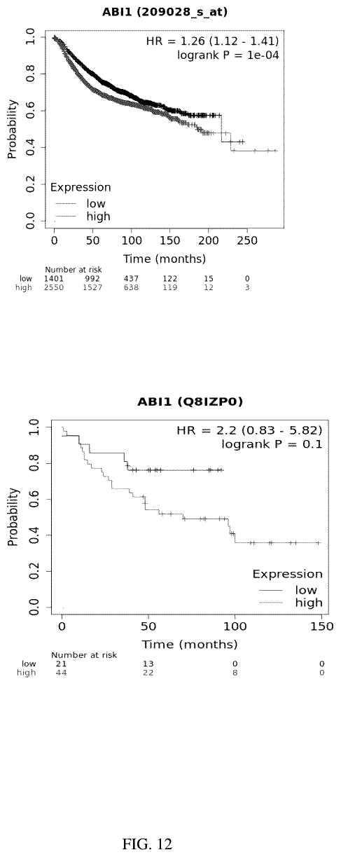

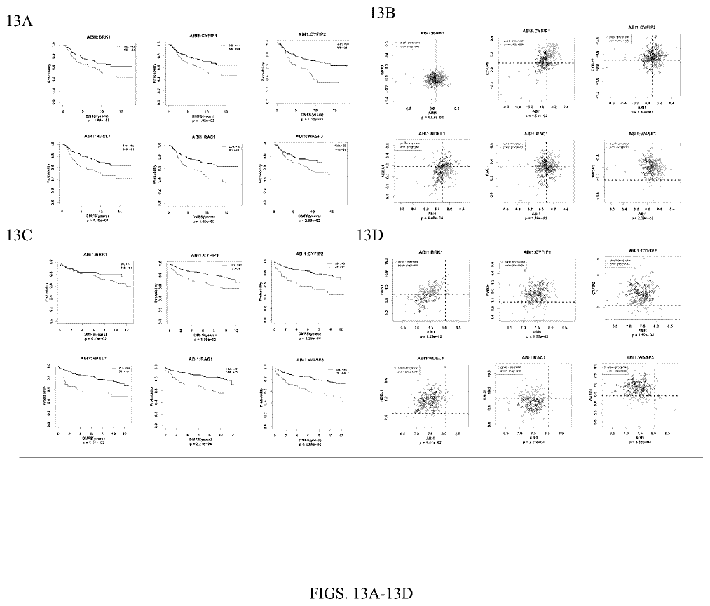

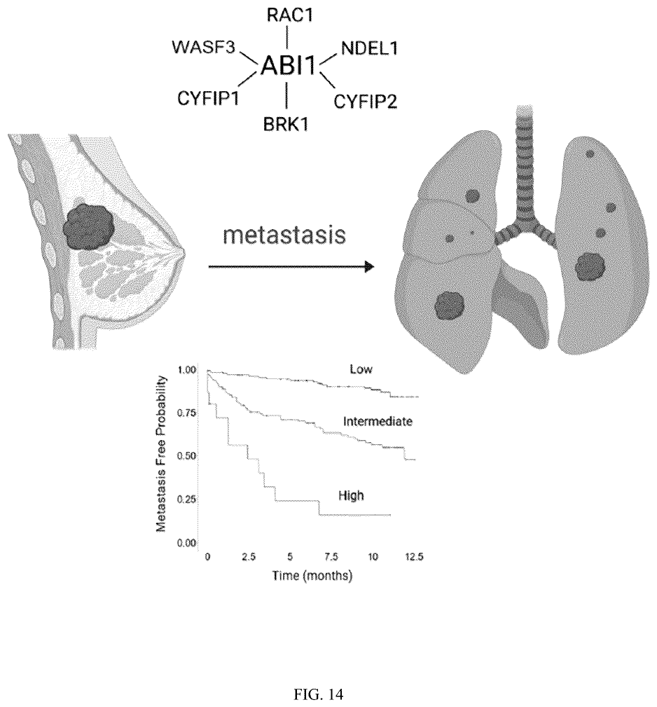

ABI1 Promotes the Number and Size of Lung Metastases in a Gene Dose-Dependent Manner. Most Abi1 KO PyMT mice demonstrated pulmonary metastasis within 6 months of the primary tumor detection. It was noted that mice with fast-growing tumors showed a positive trend for association with multiple metastatic events and large size metastatic foci in both Cre (−) control groups (FIG. 6). To elucidate the role of Abi1 gene dosage effect in lung metastasis, the tumor kinetic rates of the primary tumor growth vs. the largest tumor metastatic foci at 6 months within the same mice in Abi1 KO homozygous and heterozygous tumor groups were analyzed (FIGS. 6A-B). FIGS. 5A-B shows a weak gene dose effect in both homo- and heterozygous primary tumor kinetics. To be more conclusive, the parameters of the tumor volume kinetics were estimated using the exponential fit function ƒ (t; a, b)=(a−b*exp(ax), where parameter a is the rate of cell volume growth and (a−b) is the initial tumor volume.

No statistical differences between exponent rates in control and treatment were found. However, a comparison of the number and size of metastatic foci of Abi1 KO animals indicated a strong gene dose effect (FIGS. 6C-G). FIGS. 6C-D shows the frequency distribution of pulmonary metastatic foci that exhibited the highest number of metastatic foci and largest metastasis size in the Abi1 fl/fl and fl/wt lung tissues. In each lung sample, the frequency distribution of the metastatic foci size shows the skewed form with long tails. It was found that for each case, the frequency distribution of pulmonary metastatic foci size is fitted well by a discrete analog of shifted log-normal distribution function (for better visualization the function approximated by continuous curves) (Tables 10A-B). Estimated parameters of the distribution function were used to define significant differences between the shapes of the distribution functions shown in FIGS. 6C-D (Table 10B). In particular, parameter x₀ estimates a mode of the frequency distribution function which is most frequent size of micro-metastasis foci. For Abi1 fl/fl Cre−, fl/fwt Cre−, fl/wt Cre+ data is variated between 6.3-8.6 μm²; but for fl/fl Cre+ focus size equals 1.3 βm² (Table 10B). A comparison of x₀ and the parameter b (basal (smallest) foci size at x=0), Table 10B) of the best-fit distribution function draw in FIGS. 6C-D suggests a significant reduction of the multiple metastatic foci size and their numbers in the treatment cases fl/wt Cre+ and fl/fl Cre+. Additionally, statistical testing using the Wilcoxon Signed-Rank method demonstrated significant differences between the observed frequency distributions of treatment v.s. control datasets (p<0.0001). Comparison of the frequency distributions of the treatment groups provided a significant difference (reduction of median value in fl/fl Cre+ vs median value in fl/wt Cre+) (p<0.0001). These results indicate a strong Abi1 gene dose effect promoting lung metastases in both homozygous and heterozygous PyMT models but the effect in homozygous mice was stronger. Similar results were observed for pulmonary metastasis foci size bins (50 μm²) frequency distribution that includes all defined pulmonary metastatic foci datasets (FIGS. 6E-F). Representative lung tumor images are shown in FIG. 6G.

### Discussion

This disclosure, for the first time, demonstrates the metastasis driver role of ABI1 in breast cancer tumor progression using the PyMT mouse model and clinical data from breast cancer patients. The bioinformatics analyses revealed the significant role of human ABI1 and a subset of the WAVE complex genes in the context of breast cancer progression and metastatic process.

In the Metadata and Rosetta cohorts, the high expression of ABI1 demonstrated poor survival time patterns as indicated by survival time and is significantly associated with metastatic events. Moreover, in the large METABRIC cohort the ABI1 expression is positively correlated with DNA CNA, histologic grade 3, and basal-like phenotype, but negatively correlated with ER status and does not correlate with LN status. The present disclosure identified the high confidence and reproducible multigene survival prognosis signature comprised of ABI1 and six other genes: BRK1, CYFIP1, CYFIP2, and WASF3, which are the genes encoding WAVE complex members; and RAC1 and NDEL1 genes, which are upstream interactors and regulators of the WAVE complex [5, 62, 14]. Both RAC1 and NUDEL participate in the EMT pathway and play key roles in the metastatic migration of epithelial cells via the interaction with WAVE family proteins and the regulation of cancer-determined pathways [5, 12, 63-65].

Collectively the tumor progression and metastatic prognostic signatures allow for the identification of optimal gene expression cut-off values to stratify patients on low-, moderate- and high-risk subgroups based on DFS and DMFS times. The survival prediction analyses establish the significance of ABI1 gene expression as a pro-oncogenic factor of primary tumor formation and metastasis in breast cancer patients. These findings support the experimentally testable working hypothesis that genetic mechanisms of ABI1 are key components in the metastatic breast cancer process.

Univariate and multivariate analyses and comparisons between Kaplan-Meier survival curves generated with the prognostic signature and those generated with either estrogen receptor (ESR) or lymph node status reveal that the signature outperforms these clinically used variables and could lead to better personalized and predictable treatment selection. This conclusion is supported by the co-expression analysis between ABI1 and other members of the ABI1 survival (prediction) signature and the observed significant positive correlation between ABI1 expression, CNA, histologic grades and basal-like phenotype vs. ER (+) luminal cancer phenotype—the clinical markers of aggressiveness, metastasis and drug resistance frequency.

The availability of a genetically engineered conditional Abi1 KO mouse permitted to investigate the role of Abi1 downstream from the PyMT oncogene. By comparing the effects of one- and two-allele inactivation of the Abi1 gene, it was determined that ABI1 expression levels play an important pro-oncogenic role in breast cancer tumor progression and metastatic disease. The two-allele inactivation of the gene (in Abi1 homozygote KO mice) and one-allele inactivation of Abi1 (in Abi1 heterozygote KO mice) led to lower metastatic burden in the lungs.

Disruption of Abi1 in normal mammary epithelium led to a significant increase in terminal end buds at weeks 5 and 7 (FIG. 3B), but beyond that time point, the development of mammary glands was not affected (FIGS. 3B-D). The increase in the TEB number, as well as the trend toward increased branching in tissue with Abi1-disruption, warrants further investigation to determine whether ABI1 or other ABI proteins play a role in normal murine mammary gland development. To corroborate the findings of Abi1, the disruption of Wasf3 gene also demonstrated no significant effect on mammary gland development [22]. WASF3 is part of ABI1 7-gene signature.

The complete loss of ABI1 yields no difference in primary mammary tumor growth kinetics (FIGS. 5C, 5E) and that lung metastasis is severely abrogated in both homozygous and heterozygous Abi1 KO (FIGS. 6C-F). Thus, the findings strongly suggest that ABI1 is critical for pulmonary metastasis of aggressive breast tumors due to its essential role in sustaining WAVE complex dynamics. The WAVE complex is assembled from intimate interactions of five obligatory components: a WAVE, an ABI, a CYFIP, an NAP, and BRK protein, which are altogether products of 11 genes [6, 8, 9]. The study by Kirschner's group demonstrated that the presence of all five WAVE complex proteins is required to form the functional WAVE complex in vitro [6]. Genetic inactivation of Abi1 led to overall WAVE complex downregulation in MEF cells, but deregulation of individual WAVE complex proteins was also evident. These included the relative upregulation of ABI2. Similarly, upregulation of ABI2 is observed in breast tissue lacking ABI1 (FIGS. 4A-B). Despite their homology and similarities in function, upregulation of ABI2 cannot sustain pulmonary metastases in homozygous Abi1 KO animals (FIGS. 6C, E, G), strongly indicating that ABI1 is critical for lung metastases in this model.

The lack of local effect on primary tumor growth in ABI1 homozygous mice is difficult to explain in the context of the effect on lung metastases but raises the possibility for potential tumor suppressor role for ABI1 in breast epithelial cells in some genetic contexts such as here downstream from the PyMT oncogene. ABI1 acts as tumor suppressor in several other tissues such as prostate [30].

Focus is a pathologic term describing cells that can grow as a colony and be seen only microscopically. In the present disclosure, the differences in the number of multiple metastatic foci and the sizes of the breast cancer metastases were quantified. It was found essential differences for both characteristics in the breast cancer metastases in the ABI1 gene dosage-dependent manner. The experimental model results demonstrate the important role of ABI1 gene dosage and expression in the lung metastasis process which may model metastatic potential of CNA and gene expression of ABI1 in patient's primary breast tumors (FIG. 1A), consistent with histologic high-aggressive breast cancers (FIB. 1B), and basal-like subtype (FIG. 1D)—hallmarks of high aggressive invasive breast cancer with polyclonal metastases potential. Also, the experimental findings consist of high ABI1 protein expression in human invasive breast carcinoma associated with high risks of tumor recurrence and overall survival (FIGS. 2, 7 and 9, [32]).

It was observed that protein interaction combinations of WASF3 with some members of WAVE complex and RAC1 are responsible for breast cancer aggressiveness and metastasis [22]. In the present disclosure, it was found that an association of WASF3 and some other WAVE complex components (that are part of the prognostic signature) with invasive breast cancer that molecular pattern is associated with aggressive (basal-like) breast cancer subtype. Interestingly, heterogeneity and instability of Wave complexes without Abi1 protein could contribute to the heterogeneity in latency, the size and number of lung metastatic lesions as observed in Wasf3 KO mice [22].

The present data adhere to previously published findings regarding the impact of ABI1 protein in driving aggressive mammary oncogenesis in mouse xenograft models of breast cancer [17, 34]. ABI1 has been cited in several cancer types, such as ovarian cancer [66, 29], hepatocellular carcinoma [67], and colorectal carcinoma [68]. Notably, all studies to date examined the role of ABI1 in breast cancer using cancer cell lines. The present disclosure provides the first genetic study examining the role of Abi1 in vivo using the mouse model of aggressive breast cancer. The critical role of Abi1 in the lung metastasis in the mouse not only provides preclinical evidence for the role of Abi1 in metastatic progression but also supports ABI1-based 7-gene prognostic signature as both a prognostic marker and a prospective therapeutic target.

Univariate and multivariate analyses and comparison of Kaplan-Meier survival curves generated with the ABI1 gene expression signature to those generated with either estrogen receptor (ESR) or lymph node status reveal that the gene signature is indeed a more robust prognostic predictor than other clinically used variables and could lead to better treatment selection.

The findings of the present disclosure indicate the significant predictive value of the ABI1-based 7-gene prognostic signature derived from primary tumors in the metastatic risk of breast cancer patients. Moreover, targeting ABI1 may provide a beneficial therapeutic effect in preventing metastases.

### REFERENCES

1. Siegel R L, Miller K D & Jemal A (2020) Cancer statistics, 2020. C A Cancer J Clin 70, 7-30, doi: 10.3322/caac.21590.
2. Redig A J & McAllister S S (2013) Breast cancer as a systemic disease: a view of metastasis. J Intern Med 274, 113-126, doi: 10.1111/joim.12084.
3. Spence H J, Timpson P, Tang H R, Insall R H & Machesky L M (2012) Scar/WAVE3 contributes to motility and plasticity of lamellipodial dynamics but not invasion in three dimensions. The Biochemical journal 448, 35-42, doi: 10.1042/BJ20112206.
4. Yokotsuka M, Iwaya K, Saito T, Pandiella A, Tsuboi R, Kohno N, Matsubara O & Mukai K (2011) Overexpression of HER2 signaling to WAVE2-Arp2/3 complex activates MMP-independent migration in breast cancer. Breast Cancer Res Treat 126, 311-318, doi: 10.1007/s10549-010-0896-x.
5. Chen Z, Borek D, Padrick S B, Gomez T S, Metlagel Z, Ismail A M, Umetani J, Billadeau D D, Otwinowski Z & Rosen M K (2010) Structure and control of the actin regulatory WAVE complex. Nature 468, 533-538, Research Support, N.I.H., Extramural
Research Support, Non-U.S. Gov't
Research Support, U.S. Gov't, Non-P.H.S., doi: 10.1038/nature09623.
6. Gautreau A, Ho H Y, Li J, Steen H, Gygi S P & Kirschner M W (2004) Purification and architecture of the ubiquitous Wave complex. Proceedings of the National Academy of Sciences of the United States of America 101, 4379-4383.
7. Molinie N & Gautreau A (2018) The Arp2/3 Regulatory System and Its Deregulation in Cancer. Physiol Rev 98, 215-238, doi: 10.1152/physrev.00006.2017.
8. Miki H, Suetsugu S & Takenawa T (1998) WAVE, a novel WASP-family protein involved in actin reorganization induced by Rac. EMBO J 17, 6932-6941, doi: 10.1093/emboj/17.23.6932.
9. Suetsugu S, Miki H & Takenawa T (1999) Identification of two human WAVE/SCAR homologues as general actin regulatory molecules which associate with the Arp2/3 complex. Biochem Biophys Res Commun 260, 296-302.
10. Iwaya K, Norio K & Mukai K (2007) Coexpression of Arp2 and WAVE2 predicts poor outcome in invasive breast carcinoma. Mod Pathol 20, 339-343, doi: 10.1038/modpathol.3800741.
11. Litschko C, Linkner J, Bruhmann S, Stradal T E B, Reinl T, Jansch L, Rottner K & Faix J (2017) Differential functions of WAVE regulatory complex subunits in the regulation of actin-driven processes. Eur J Cell Biol 96, 715-727, doi: 10.1016/j.ejcb.2017.08.003.
12. Eden S, Rohatgi R, Podtelejnikov A V, Mann M & Kirschner M W (2002) Mechanism of regulation of WAVE1-induced actin nucleation by Rac1 and Nck. Nature 418, 790-793, doi: 10.1038/nature00859.
13. Lebensohn A M & Kirschner M W (2009) Activation of the WAVE complex by coincident signals controls actin assembly. Mol Cell 36, 512-524, doi: 10.1016/j.molcel.2009.10.024.
14. Wu S, Ma L, Wu Y, Zeng R & Zhu X (2012) Nudel is crucial for the WAVE complex assembly in vivo by selectively promoting subcomplex stability and formation through direct interactions. Cell Res 22, 1270-1284, doi: 10.1038/cr.2012.47.
15. Dubielecka P M, Cui P, Xiong X, Hossain S, Heck S, Angelov L & Kotula L (2010) Differential regulation of macropinocytosis by Abi1/Hssh3bp1 isoforms. PloS one 5, e10430, doi: 10.1371/journal.pone.0010430.
16. Dubielecka P M, Ladwein K I, Xiong X, Migeotte I, Chorzalska A, Anderson K V, Sawicki J A, Rottner K, Stradal T E & Kotula L (2011) Essential role for Abi1 in embryonic survival and WAVE2 complex integrity. Proceedings of the National Academy of Sciences of the United States of America 108, 7022-7027, doi: 10.1073/pnas.1016811108.
17. Innocenti M, Zucconi A, Disanza A, Frittoli E, Areces L B, Steffen A, Stradal T E, Di Fiore P P, Carlier M F & Scita G (2004) Abi1 is essential for the formation and activation of a WAVE2 signalling complex. Nature cell biology 6, 319-327, doi: 10.1038/ncb1105.
18. Roffers-Agarwal J, Xanthos J B & Miller J R (2005) Regulation of actin cytoskeleton architecture by Eps8 and Abi1. BMC Cell Biol 6, 36, doi: 10.1186/1471-2121-6-36.
19. Sweeney M O, Collins A, Padrick S B & Goode B L (2015) A novel role for WAVE1 in controlling actin network growth rate and architecture. Mol Biol Cell 26, 495-505, doi: 10.1091/mbc.E14-10-1477.
20. Tang Q, Schaks M, Koundinya N, Yang C, Pollard L W, Svitkina T M, Rottner K & Goode B L (2020) WAVE1 and WAVE2 have distinct and overlapping roles in controlling actin assembly at the leading edge. Mol Biol Cell 31, 2168-2178, doi: 10.1091/mbc.E19-12-0705.
21. Loveless R & Teng Y (2021) Targeting WASF3 Signaling in Metastatic Cancer. Int J Mol Sci 22, doi: 10.3390/ijms22020836.
22. Qin H, Lu S, Thangaraju M & Cowell J K (2019) Wasf3 Deficiency Reveals Involvement in Metastasis in a Mouse Model of Breast Cancer. The American journal of pathology 189, 2450-2458, doi: 10.1016/j.ajpath.2019.08.012.
23. Teng Y, Ngoka L & Cowell J K (2017) Promotion of invasion by mutant RAS is dependent on activation of the WASF3 metastasis promoter gene. Genes Chromosomes Cancer 56, 493-500, doi: 10.1002/gcc.22453.
24. Teng Y, Qin H, Bahassan A, Bendzunas N G, Kennedy E J & Cowell J K (2016) The WASF3-NCKAP1-CYFIP1 Complex Is Essential for Breast Cancer Metastasis. Cancer Res 76, 5133-5142, doi: 10.1158/0008-5472.Can-16-0562.
25. Molinie N, Rubtsova S N, Fokin A, Visweshwaran S P, Rocques N, Polesskaya A, Schnitzler A, Vacher S, Denisov E V, Tashireva L A, Perelmuter V M, Cherdyntseva N V, Bieche I & Gautreau A M (2019) Cortical branched actin determines cell cycle progression. Cell Res 29, 432-445, doi: 10.1038/s41422-019-0160-9.
26. Chorzalska A, Morgan J, Ahsan N, Treaba D O, Olszewski A J, Petersen M, Kingston N, Cheng Y, Lombardo K, Schorl C, Yu X, Zini R, Pacilli A, Tepper A, Coburn J, Hryniewicz-Jankowska A, Zhao T C, Oancea E, Reagan J L, Liang O, Kotula L, Quesenberry P J, Gruppuso P A, Manfredini R, Vannucchi A M & Dubielecka P M (2018) Bone marrow-specific loss of ABI1 induces myeloproliferative neoplasm with features resembling human myelofibrosis. Blood 132, 2053-2066, doi: 10.1182/blood-2018-05-848408.
27. Kumar S, Lu B, Dixit U, Hossain S, Liu Y, Li J, Hornbeck P, Zheng W, Sowalsky A G, Kotula L & Birge R B (2015) Reciprocal regulation of Abl kinase by Crk Y251 and Abi1 controls invasive phenotypes in glioblastoma. Oncotarget 6, 37792-37807, doi: 10.18632/oncotarget.6096.
28. Steinestel K, Bruderlein S, Steinestel J, Markl B, Schwerer M J, Arndt A, Kraft K, Propper C & Moller P (2012) Expression of Abelson interactor 1 (Abi1) correlates with inflammation, KRAS mutation and adenomatous change during colonic carcinogenesis. PloS one 7, e40671, doi: 10.1371/journal.pone.0040671.
29. Zhang J, Tang L, Chen Y, Duan Z, Xiao L, Li W, Liu X & Shen L (2015) Upregulation of Abelson interactor protein 1 predicts tumor progression and poor outcome in epithelial ovarian cancer. Hum Pathol 46, 1331-1340, doi: 10.1016/j.humpath.2015.05.015.
30. Nath D, Li X, Mondragon C, Post D, Chen M, White J R, Hryniewicz-Jankowska A, Caza T, Kuznetsov V A, Hehnly H, Jamaspishvili T, Berman D M, Zhang F, Kung S H Y, Fazli L, Gleave M E, Bratslavsky G, Pandolfi P P & Kotula L (2019) Abi1 loss drives prostate tumorigenesis through activation of EMT and non-canonical WNT signaling. Cell communication and signaling: CCS 17, 120, doi: 10.1186/s12964-019-0410-y.
31. Xiong X, Chorzalska A, Dubielecka P M, White J R, Vedvyas Y, Hedvat C V, Haimovitz-Friedman A, Koutcher J A, Reimand J, Bader G D, Sawicki J A & Kotula L (2012) Disruption of Abi1/Hssh3bp1 expression induces prostatic intraepithelial neoplasia in the conditional Abi1/Hssh3bp1 K O mice. Oncogenesis 1, e26, doi: 10.1038/oncsis.2012.28.
32. Wang C, Tran-Thanh D, Moreno J C, Cawthorn T R, Jacks L M, Wang D Y, McCready D R & Done S J (2011) Expression of Abl interactor 1 and its prognostic significance in breast cancer: a tissue-array-based investigation. Breast Cancer Res Treat 129, 373-386, doi: 10.1007/s10549-010-1241-0.
33. Sun X, Li C, Zhuang C, Gilmore W C, Cobos E, Tao Y & Dai Z (2009) Abl interactor 1 regulates Src-Id1-matrix metalloproteinase 9 axis and is required for invadopodia formation, extracellular matrix degradation and tumor growth of human breast cancer cells. Carcinogenesis 30, 2109-2116, doi: 10.1093/carcin/bgp251.
34. Wang C, Navab R, Iakovlev V, Leng Y, Zhang J, Tsao M S, Siminovitch K, McCready D R & Done S J (2007) Abelson interactor protein-1 positively regulates breast cancer cell proliferation, migration, and invasion. Molecular cancer research: MCR 5, 1031-1039, doi: 10.1158/1541-7786.MCR-06-0391.
35. Fluck M M & Schaffhausen B S (2009) Lessons in signaling and tumorigenesis from polyomavirus middle T antigen. Microbiology and molecular biology reviews: MMBR 73, 542-563, doi: 10.1128/MMBR.00009-09.
36. Lin E Y, Jones J G, Li P, Zhu L, Whitney K D, Muller W J & Pollard J W (2003) Progression to malignancy in the polyoma middle T oncoprotein mouse breast cancer model provides a reliable model for human diseases. The American journal of pathology 163, 2113-2126, Research Support, Non-U.S. Gov't Research Support, U.S. Gov't, P.H.S., doi: 10.1016/50002-9440(10)63568-7.
37. Chen L, Jenjaroenpun P, Pillai A M, Ivshina A V, Ow G S, Efthimios M, Zhiqun T, Tan T Z, Lee S C, Rogers K, Ward J M, Mori S, Adams D J, Jenkins N A, Copeland N G, Ban K H, Kuznetsov V A & Thiery J P (2017) Transposon insertional mutagenesis in mice identifies human breast cancer susceptibility genes and signatures for stratification. Proc Natl Acad Sci USA 114, E2215-2224, doi: 10.1073/pnas.1701512114.
38. Kulkarni S, Augoff K, Rivera L, McCue B, Khoury T, Groman A, Zhang L, Tian L & Sossey-Alaoui K (2012) Increased expression levels of WAVE3 are associated with the progression and metastasis of triple negative breast cancer. PloS one 7, e42895, Research Support, N.I.H., Extramural
Research Support, Non-U.S. Gov't
Research Support, U.S. Gov't, Non-P.H.S., doi: 10.1371/journal.pone.0042895.
39. van′t Veer L J, Dai H, van de Vijver M J, He Y D, Hart A A, Mao M, Peterse H L, van der Kooy K, Marton M J, Witteveen A T, Schreiber G J, Kerkhoven R M, Roberts C, Linsley P S, Bernards R & Friend S H (2002) Gene expression profiling predicts clinical outcome of breast cancer. Nature 415, 530-536, doi: 10.1038/415530a.
40. Ivshina A V, George J, Senko O, Mow B, Putti T C, Smeds J, Lindahl T, Pawitan Y, Hall P, Nordgren H, Wong J E, Liu E T, Bergh J, Kuznetsov V A & Miller L D (2006) Genetic reclassification of histologic grade delineates new clinical subtypes of breast cancer. Cancer Res 66, 10292-10301, doi: 10.1158/0008-5472.Can-05-4414.
41. Miller L D, Smeds J, George J, Vega V B, Vergara L, Ploner A, Pawitan Y, Hall P, Klaar S, Liu E T & Bergh J (2005) An expression signature for p53 status in human breast cancer predicts mutation status, transcriptional effects, and patient survival. Proc Natl Acad Sci USA 102, 13550-13555, doi: 10.1073/pnas.0506230102.
42. Curtis C, Shah S P, Chin S F, Turashvili G, Rueda O M, Dunning M J, Speed D, Lynch A G, Samarajiwa S, Yuan Y, Gräf S, Ha G, Haffari G, Bashashati A, Russell R, McKinney S, Langerød A, Green A, Provenzano E, Wishart G, Pinder S, Watson P, Markowetz F, Murphy L, Ellis I, Purushotham A, Bsrresen-Dale A L, Brenton J D, Tavard S, Caldas C & Aparicio S (2012) The genomic and transcriptomic architecture of 2,000 breast tumours reveals novel subgroups. Nature 486, 346-352, doi: 10.1038/nature10983.
43. de Carvalho L P, Tan S H, Ow G-S, Tang Z, Ching J, Kovalik J-P, Poh S C, Chin C-T, Richards A M, Martinez E C, Troughton R W, Fong A Y-Y, Yan B P, Seneviratna A, Sorokin V, Summers S A, Kuznetsov V A & Chan M Y (2018) Plasma Ceramides as Prognostic Biomarkers and Their Arterial and Myocardial Tissue Correlates in Acute Myocardial Infarction. JACC: Basic to Translational Science 3, 163-175, doi: 10.1016/j.jacbts.2017.12.005.
44. Kuznetsov V A, Tang Z & Ivshina A V (2017) Identification of common oncogenic and early developmental pathways in the ovarian carcinomas controlling by distinct prognostically significant microRNA subsets. BMC Genomics 18, 692, doi: 10.1186/s12864-017-4027-5.
45. Motakis E, Ivshina A V & Kuznetsov V A (2009) Data-driven approach to predict survival of cancer patients: estimation of microarray genes' prediction significance by Cox proportional hazard regression model. IEEE Eng Med Biol Mag 28, 58-66, doi: 10.1109/memb.2009.932937.
46. Enderlein G (1987) Cox, D. R.; Oakes, D.: Analysis of Survival Data. Chapman and Hall, London-New York 1984, 201 S., £12,-. Biometrical Journal 29, 114-114, doi: https://doi.org/10.1002/bimj.4710290119.
47. Breslow N (1974) Covariance analysis of censored survival data. Biometrics 30, 89-99.
48. Chen L, Jenjaroenpun P, Pillai A M, Ivshina A V, Ow G S, Efthimios M, Zhiqun T, Tan T Z, Lee S C, Rogers K, Ward J M, Mori S, Adams D J, Jenkins N A, Copeland N G, Ban K H, Kuznetsov V A & Thiery J P (2017) Transposon insertional mutagenesis in mice identifies human breast cancer susceptibility genes and signatures for stratification. Proceedings of the National Academy of Sciences of the United States of America 114, E2215-e2224, doi: 10.1073/pnas.1701512114.
49. Kuznetsov V A, Tang Z & Ivshina A V (2017) Identification of common oncogenic and early developmental pathways in the ovarian carcinomas controlling by distinct prognostically significant microRNA subsets. BMC Genomics 18, 692, doi: 10.1186/s12864-017-4027-5.
50. Euhus D M, Hudd C, LaRegina M C & Johnson F E (1986) Tumor measurement in the nude mouse. J Surg Oncol 31, 229-234, doi: 10.1002/jso.2930310402.
51. Thorpe L M, Spangle J M, Ohlson C E, Cheng H, Roberts T M, Cantley L C & Zhao J J (2017) PI3K-p110alpha mediates the oncogenic activity induced by loss of the novel tumor suppressor PI3K-p85alpha. Proceedings of the National Academy of Sciences of the United States of America 114, 7095-7100, doi: 10.1073/pnas.1704706114.
52. Plante I, Stewart M K & Laird D W (2011) Evaluation of mammary gland development and function in mouse models. J Vis Exp 53, 2828, doi: 10.3791/2828.
53. Roarty K & Serra R (2007) Wnt5a is required for proper mammary gland development and TGF-beta-mediated inhibition of ductal growth. Development 134, 3929-3939, doi: 10.1242/dev.008250.
54. Wali V B, Gilmore-Hebert M, Mamillapalli R, Haskins J W, Kurppa K J, Elenius K, Booth C J & Stern D F (2014) Overexpression of ERBB4 JM-a CYT-1 and CYT-2 isoforms in transgenic mice reveals isoform-specific roles in mammary gland development and carcinogenesis. Breast Cancer Res 16, 501, doi: 10.1186/s13058-014-0501-z.
55. Frugtniet B, Jiang W G & Martin T A (2015) Role of the WASP and WAVE family proteins in breast cancer invasion and metastasis. Breast Cancer 7, 99-109, doi: 10.2147/BCTT.S59006.
56. Aswad L, Yenamandra S P, Siong Ow G, Grinchuk O, Ivshina A V & Kuznetsov V A (2015) Genome and transcriptome delineation of two major oncogenic pathways governing invasive ductal breast cancer development. Oncotarget 6.
57. Kuznetsov V A, Motakis E & Ivshina A V, 2008. Low- and high-agressive genetic breast cancer subtypes and significant survival gene signatures, 2008 IEEE International Joint Conference on Neural Networks (IEEE World Congress on Computational Intelligence), pp. 4151-4156.
58. Inman J L, Robertson C, Mott J D & Bissell M J (2015) Mammary gland development: cell fate specification, stem cells and the microenvironment. Development 142, 1028-1042, doi: 10.1242/dev.087643.
59. Wagner K, McAllister K, Ward T, Davis B, Wiseman R & Hennighausen L (2001) Spatial and temporal expression of the Cre gene under the control of the MMTV-LTR in different lines of transgenic mice. Transgenic Res 10, 545-553.
60. Ouderkirk-Pecone J L, Goreczny G J, Chase S E, Tatum A H, Turner C E & Krendel M (2016) Myosin 1e promotes breast cancer malignancy by enhancing tumor cell proliferation and stimulating tumor cell de-differentiation. Oncotarget 7, 46419-46432, doi: 10.18632/oncotarget.10139.
61. Kurisu S & Takenawa T (2009) The WASP and WAVE family proteins. Genome Biol 10, 226, doi: 10.1186/gb-2009-10-6-226.
62. Niethammer M, Smith D S, Ayala R, Peng J, Ko J, Lee M S, Morabito M & Tsai L H (2000) NUDEL is a novel Cdk5 substrate that associates with LIS1 and cytoplasmic dynein. Neuron 28, 697-711, doi: 10.1016/s0896-6273(00)00147-1.
63. Fang D, Chen H, Zhu J Y, Wang W, Teng Y, Ding H F, Jing Q, Su S B & Huang S (2017) Epithelial-mesenchymal transition of ovarian cancer cells is sustained by Rac1 through simultaneous activation of MEK1/2 and Src signaling pathways. Oncogene 36, 1546-1558, doi: 10.1038/onc.2016.323.
64. Kaneto N, Yokoyama S, Hayakawa Y, Kato S, Sakurai H & Saiki I (2014) RAC1 inhibition as a therapeutic target for gefitinib-resistant non-small-cell lung cancer. Cancer Sci 105, 788-794, doi: 10.1111/cas.12425.
65. Zhang W, Xing L, Xu L, Jin X, Du Y, Feng X, Liu S & Liu Q (2019) Nudel involvement in the high-glucose-induced epithelial-mesenchymal transition of tubular epithelial cells. Am J Physiol Renal Physiol 316, F186-f194, doi: 10.1152/ajprenal.00218.2018.
66. Yu X, Liang C, Zhang Y, Zhang W & Chen H (2019) Inhibitory short peptides targeting EPS8/ABI1/SOS1 tri-complex suppress invasion and metastasis of ovarian cancer cells. BMC Cancer 19, 878, doi: 10.1186/s12885-019-6087-1.
67. Wang J L, Yan T T, Long C & Cai W W (2017) Oncogenic function and prognostic significance of Abelson interactor 1 in hepatocellular carcinoma. Int J Oncol 50, 1889-1898, doi: 10.3892/ijo.2017.3920.
68. Steinestel K, Bruderlein S, Lennerz J K, Steinestel J, Kraft K, Propper C, Meineke V & Moller P (2014) Expression and Y435-phosphorylation of Abelson interactor 1 (Abi1) promotes tumour cell adhesion, extracellular matrix degradation and invasion by colorectal carcinoma cells. Mol Cancer 13, 145, doi: 10.1186/1476-4598-13-145.

### Tables

TABLE 1
Gene expression variability upon Abi1 depletion in primary
PyMT breast cancer tumors defined by RNA-seq.

| Mouse ID | | G144 | G164 | G184 | G174 | Fold Change Depletion | | | |
|---|---|---|---|---|---|---|---|---|---|
| Genotype | | fl/wt | fl/wt | fl/fl | fl/fl | Hetero- | Homo- | | Treatment |
| Cre | | − | + | − | + | zygous | zygous | Ratio | Effect* |
| Gene | Abi1 | 14389 | 7343 | 15682 | 1018 | 1.96 | 15.40 | 7.86 | yes |
| expression | Abi2 | 8173 | 7189 | 8031 | 9719 | 1.14 | 0.83 | 0.73 | no |
| (RNA-seq | Abi3 | 146 | 271 | 343 | 195 | 0.54 | 1.76 | 3.26 | yes |
| from | Nckap1 | 36435 | 34964 | 40591 | 38802 | 1.04 | 1.05 | 1.00 | no |
| primary | Wasf1 | 88 | 66 | 85 | 118 | 1.33 | 0.72 | 0.54 | no |
| tumors) | Wasf2 | 8563 | 10349 | 13894 | 11087 | 0.83 | 1.25 | 1.51 | no |
| | Wasf3 | 261 | 151 | 95 | 221 | 1.73 | 0.43 | 0.25 | yes |
| | Brk1 | 10240 | 9103 | 11091 | 9920 | 1.12 | 1.12 | 0.99 | no |
| | Cyfip1 | 23448 | 22528 | 31708 | 23030 | 1.04 | 1.38 | 1.32 | no |
| | Cyfip2 | 521 | 1289 | 1633 | 580 | 0.40 | 2.82 | 6.97 | yes |
| | Rac1 | 25732 | 22621 | 27209 | 24431 | 1.14 | 1.11 | 0.98 | no |
| | Ndel1 | 10923 | 10842 | 15082 | 13030 | 1.01 | 1.16 | 1.15 | no |

*Treatment effect is “positive” (yes) if the fold change of gene expression for heterozygous and homozygous mice changed more than 1.5 times in any direction and “negative” (no) in other cases. RNA-seq. expression profiles of WAVE complex, and Rac1 and Ndel1 genes (involved in WAVE complex stability and functionality) show the differences between heterozygous vs. homozygous Abi1 KO PyMT mammary tumors.

TABLE 2
Gene list for prognostic signatures and associated probes/probsets representing by Rosetta (Merk) and Affymetrix U133-A & B microarrays.

| Gene | Refseq | Entrez | Ensembl | | | Rosetta | Affy |
|---|---|---|---|---|---|---|---|
| Symbol | ID | ID | ID | Description | Gene_chrom | probe IDs | probe IDs |
| ABI1 | NM_005470 | 10006 | ENSG00000136754 | abI-interactor 1 | chr10 | 10012674234 | 209027_s_at |
| BRK1 | NM_018462 | 55845 | ENSG00000254999 | BRICK1 Subunit Of SCAR/WAVE | chr3 | 10012685694 | 224575_at |
| | | | | Actin Nucleating Complex | | | |
| CYFIP1 | NM_014608 | 23191 | ENSG00000273749 | cytoplasmic FMR1 interacting | chr15 | 10012680036 | 208923_at |
| | | | | protein 1 | | | |
| CYFIP2 | NM_001037333 | 26999 | ENSG00000055163 | cytoplasmic FMR1 interacting | chr5 | 10012678764 | 215785_s_at |
| | | | | protein 2 | | | |
| NDEL1 | NM_001025579 | 81565 | ENSG00000166579 | Nuclear Distribution | chr17 | 10012684491 | 208093_s_at |
| | | | | Protein NudE-Like 1 | | | |
| RAC1 | NM_006908 | 5879 | ENSG00000136238 | Rac Family Small | chr7 | 10012688805 | 208641_s_at |
| | | | | GTPase 1 | | | |
| WASF3 | NM_006646 | 10810 | ENSG00000132970 | WAS protein family, | chr13 | 10012678244 | 204042_at |
| | | | | member 3 | | | |

Some genes were represented by more then one probes/probsets; only one most reliable an significal prob set was selected.

TABLE 3
Results of 1D DDg survival prediction (DFS and DMFS) for Rosetta and Metadata sets

| | | | | | | | |
|---|---|---|---|---|---|---|---|
| Rosetta | | | Entrez | | p-value | | |
| probe IDs | Gene | Refseq | ID | Description | (Wald test) | Rank | Weights |

Rosetta_DFS

| | | | | | | | |
|---|---|---|---|---|---|---|---|
| 10012674234 | ABI1 | NM_005470 | 10006 | abI-interactor 1 | 0.0251 | 5 | 0.121 |
| 10012680036 | CYFIP1 | NM_014608 | 23191 | cytoplasmic FMR1 | 0.0084 | 3 | 0.156 |
| | | | | interacting protein 1 | | | |
| 10012678764 | CYFIP2 | NM_001037333 | 26999 | cytoplasmic FMR1 | 0.0028 | 2 | 0.193 |
| | | | | interacting protein 2 | | | |
| 10012678244 | WASF3 | NM_006646 | 10810 | WAS protein family, | 0.1100 | 7 | 0.072 |
| | | | | member 3 | | | |
| 10012685694 | BRK1 | NM_018462 | 55845 | BRICK1 Subunit Of | 0.0517 | 6 | 0.097 |
| | | | | SCAR/WAVE Actin | | | |
| | | | | Nucleating Complex | | | |
| 10012688805 | RAC1 | NM_006908 | 5879 | ras-related C3 | 0.0015 | 1 | 0.213 |
| | | | | botulinum toxin | | | |
| | | | | substrate 1 (rho | | | |
| | | | | family, small GTP | | | |
| | | | | binding protein Rac1) | | | |
| 10012684491 | NDEL1 | NM_001025579 | 81565 | nudE nuclear | 0.0109 | 4 | 0.148 |
| | | | | distribution gene E | | | |
| | | | | homolog (A. | | | |
| | | | | nidulans)-like 1 | | | |

Rosetta_DMFS

| | | | | | | | |
|---|---|---|---|---|---|---|---|
| 10012674234 | ABI1 | NM_005470 | 10006 | abI-interactor 1 | 0.061 | 6 | 0.089 |
| 10012680036 | CYFIP1 | NM_014608 | 23191 | cytoplasmic FMR1 | 0.005 | 3 | 0.173 |
| | | | | interacting protein 1 | | | |
| 10012678764 | CYFIP2 | NM_001037333 | 26999 | cytoplasmic FMR1 | 0.0016 | 2 | 0.209 |
| | | | | interacting protein 2 | | | |
| 10012678244 | WASF3 | NM_006646 | 10810 | WAS protein family, | 0.126 | 7 | 0.067 |
| | | | | member 3 | | | |
| 10012685694 | BRK1 | NM_018462 | 55845 | BRICK1 Subunit Of | 0.026 | 5 | 0.118 |
| | | | | SCAR/WAVE Actin | | | |
| | | | | Nucleating Complex | | | |
| 10012688805 | RAC1 | NM_006908 | 5879 | ras-related C3 | 0.0014 | 1 | 0.212 |
| | | | | botulinum toxin | | | |
| | | | | substrate 1 (rho | | | |
| | | | | family, small GTP | | | |
| | | | | binding protein Rac1) | | | |
| 10012684491 | NDEL1 | NM_001025579 | 81565 | nudE nuclear | 0.018 | 4 | 0.131 |
| | | | | distribution gene E | | | |
| | | | | homolog (A. | | | |
| | | | | nidulans)-like 1 | | | |

Metadata_DFS

| | | | | | | | |
|---|---|---|---|---|---|---|---|
| 209027_s_at | ABI1 | NM_005470 | 10006 | abI-interactor 1 | 0.0083 | 4 | 0.142 |
| 208923_at | CYFIP1 | NM_014608 | 23191 | cytoplasmic FMR1 | 0.0018 | 2 | 0.188 |
| | | | | interacting protein 1 | | | |
| 215785_s_at | CYFIP2 | NM_001037333 | 26999 | cytoplasmic FMR1 | 0.0074 | 3 | 0.146 |
| | | | | interacting protein 2 | | | |
| 204042_at | WASF3 | NM_006646 | 10810 | WAS protein family, | 0.0102 | 5 | 0.136 |
| | | | | member 3 | | | |
| 224575_at | BRK1 | NM_018462 | 55845 | BRICK1 Subunit Of | 0.0330 | 6 | 0.101 |
| | | | | SCAR/WAVE Actin | | | |
| | | | | Nucleating Complex | | | |
| 208641_s_at | RAC1 | NM_006908 | 5879 | ras-related C3 | 0.0005 | 1 | 0.226 |
| | | | | botulinum toxin | | | |
| | | | | substrate 1 (rho | | | |
| | | | | family, small GTP | | | |
| | | | | binding protein Rac1) | | | |
| 208093_s_at | NDEL1 | NM_001025579 | 81565 | nudE nuclear | 0.1343 | 7 | 0.060 |
| | | | | distribution gene E | | | |
| | | | | homolog (A. | | | |
| | | | | nidulans)-like 1 | | | |

Metadata_DMFS

| | | | | | | | |
|---|---|---|---|---|---|---|---|
| 209027_s_at | ABI1 | NM_005470 | 10006 | abI-interactor 1 | 0.023 | 4 | 0.105 |
| 208923_at | CYFIP1 | NM_014608 | 23191 | cytoplasmic FMR1 | 0.069 | 7 | 0.074 |
| | | | | interacting protein 1 | | | |
| 215785_s_at | CYFIP2 | NM_001037333 | 26999 | cytoplasmic FMR1 | 0.00015 | 1 | 0.246 |
| | | | | interacting protein 2 | | | |
| 204042_at | WASF3 | NM_006646 | 10810 | WAS protein family, | 0.002 | 3 | 0.178 |
| | | | | member 3 | | | |
| 224575_at | BRK1 | NM_018462 | 55845 | BRICK1 Subunit Of | 0.057 | 6 | 0.080 |
| | | | | SCAR/WAVE Actin | | | |
| | | | | Nucleating Complex | | | |
| 208641_s_at | RAC1 | NM_006908 | 5879 | ras-related C3 | 0.00028 | 2 | 0.228 |
| | | | | botulinum toxin | | | |
| | | | | substrate 1 (rho | | | |
| | | | | family, small GTP | | | |
| | | | | binding protein Rac1) | | | |
| 208093_s_at | NDEL1 | NM_001025579 | 81565 | nudE nuclear | 0.043 | 5 | 0.088 |
| | | | | distribution gene E | | | |
| | | | | homolog (A. | | | |
| | | | | nidulans)-like 1 | | | |

| Rosetta | | model | mean | mean | p-value | n | n |
|---|---|---|---|---|---|---|---|
| probe IDs | cutoff | design | group | gro [symbol] | of [symbol] | group 1 | group 2 |
| Rosetta_DFS | | | | | | | |
| 10012674234 | 0.153 | Oncogene- | 0.039 | 0.217 | 3.09E−41 | 210 | 85 |
| | | like | | | | | |
| 10012680036 | 0.089 | Oncogene- | 0.026 | 0.203 | 7.00E−39 | 77 | 218 |
| | | like | | | | | |
| 10012678764 | −0.689 | Tumor | −0.595 | −0.773 | 7.00E−46 | 190 | 105 |
| | | suppressor- | | | | | |
| | | like | | | | | |
| 10012678244 | −1.077 | Tumor | −0.923 | −1.188 | 2.63E−42 | 206 | 89 |
| | | suppressor- | | | | | |
| | | like | | | | | |
| 10012685694 | −0.456 | Oncogene- | −0.556 | −0.374 | 7.98E−21 | 262 | 33 |
| | | like | | | | | |
| 10012688805 | 0.347 | Oncogene- | 0.267 | 0.418 | 9.45E−50 | 140 | 155 |
| | | like | | | | | |
| 10012684491 | 0.307 | Tumor | 0.358 | 0.220 | 3.05E−36 | 69 | 226 |
| | | suppressor- | | | | | |
| | | like | | | | | |
| Rosetta_DMFS | | | | | | | |
| 10012674234 | 0.194 | Oncogene- | 0.057 | 0.245 | 1.10E−29 | 243 | 52 |
| | | like | | | | | |
| 10012680036 | 0.089 | Oncogene- | 0.026 | 0.203 | 7.00E−39 | 77 | 218 |
| | | like | | | | | |
| 10012678764 | −0.689 | Tumor | −0.595 | −0.773 | 7.00E−46 | 190 | 105 |
| | | suppressor- | | | | | |
| | | like | | | | | |
| 10012678244 | −1.268 | Tumor | −0.982 | −1.376 | 1.76E−11 | 279 | 16 |
| | | suppressor- | | | | | |
| | | like | | | | | |
| 10012685694 | −0.456 | Oncogene- | −0.556 | −0.374 | 7.98E−21 | 262 | 33 |
| | | like | | | | | |
| 10012688805 | 0.347 | Oncogene- | 0.267 | 0.418 | 9.45E−50 | 140 | 155 |
| | | like | | | | | |
| 10012684491 | 0.295 | Tumor | 0.345 | 0.212 | 2.63E−42 | 89 | 206 |
| | | suppressor- | | | | | |
| | | like | | | | | |
| Metadata_DFS | | | | | | | |
| 209027_s_at | 8.040 | Oncogene- | 7.42 | 8.18 | 1.31E−09 | 236 | 13 |
| | | like | | | | | |
| 208923_at | 9.110 | Oncogene- | 8.85 | 9.22 | 3.60E−23 | 210 | 39 |
| | | like | | | | | |
| 215785_s_at | 6.320 | Tumor | 7.02 | 5.97 | 6.09E−29 | 196 | 53 |
| | | supressor- | | | | | |
| | | like | | | | | |
| 204042_at | 7.300 | Oncogene- | 6.71 | 7.51 | 2.47E−16 | 224 | 25 |
| | | like | | | | | |
| 224575_at | 9.040 | Oncogene- | 8.83 | 9.41 | 6.04E−29 | 53 | 196 |
| | | like | | | | | |
| 208641_s_at | 9.810 | Oncogene- | 9.59 | 10.01 | 2.04E−39 | 158 | 91 |
| | | like | | | | | |
| 208093_s_at | 7.100 | Tumor | 7.48 | 7.02 | 1.23E−13 | 229 | 20 |
| | | supressor- | | | | | |
| | | like | | | | | |
| Metadata_DMFS | | | | | | | |
| 209027_s_at | 8.040 | Oncogene- | 7.42 | 8.18 | 1.31E−09 | 236 | 13 |
| | | like | | | | | |
| 208923_at | 8.750 | Tumor | 8.99 | 8.65 | 1.89E−31 | 189 | 60 |
| | | supressor- | | | | | |
| | | like | | | | | |
| 215785_s_at | 6.110 | Tumor | 6.95 | 5.83 | 2.55E−21 | 214 | 35 |
| | | supressor- | | | | | |
| | | like | | | | | |
| 204042_at | 6.420 | Tumor | 6.96 | 6.11 | 8.58E−28 | 199 | 50 |
| | | supressor- | | | | | |
| | | like | | | | | |
| 224575_at | 9.210 | Oncogene- | 8.94 | 9.46 | 7.67E−38 | 83 | 166 |
| | | like | | | | | |
| 208641_s_at | 9.780 | Oncogene- | 9.58 | 9.99 | 1.18E−40 | 150 | 99 |
| | | like | | | | | |
| 208093_s_at | 7.100 | Tumor | 7.48 | 7.02 | 1.23E−13 | 22 | 20 |
| | | supressor- | | | | | |
| | | like | | | | | |

[symbol]

indicates data missing or illegible when filed

TABLE 4
1D-DDg by ABI1 expression level suggests significant metastasis events enrichment in high risk group (DMFS time)
A: Rosetta

| | | | 10012674234 | ABI1 (1: no | | |
|---|---|---|---|---|---|---|
| | Metastasis | EVENT: | (expression | metastases; 2: | | |
| Sample_id | TIME(year) | metastais | signal) | metasatases) | Gene | ABI1 |
| 123 | 14.261465 | 0 | 0.222723048 | 2 | Probe | 10012674234 |
| 135 | 9.330595 | 0 | 0.306723048 | 2 | Refseq | NM_005470 |
| 140 | 5.555099 | 0 | 0.202723048 | 2 | EntrezID | 10006 |
| 147 | 1.609856 | 1 | 0.217723048 | 2 | Description | abI-interactor 1 |
| 148 | 18.340862 | 0 | 0.260723048 | 2 | K-M: p-value | [symbol] |
| | | | | | (Log-rank test) | |
| 150 | 0.960986 | 1 | 0.198723048 | 2 | Rank | 6 |
| 151 | 14.01232 | 1 | 0.302723048 | 2 | Weights | 0.089 |
| 159 | 4.44627 | 1 | 0.198723048 | 2 | cutoff | 0.194 |
| 163 | 15.819302 | 0 | 0.219723048 | 2 | survival model | [symbol] |
| | | | | | design | |
| 164 | 5.664613 | 0 | 0.217723048 | 2 | mean_group1 | 0.0573 |
| 166 | 1.612594 | 1 | 0.202723048 | 2 | mean_group2 | 0.2451 |
| 167 | 15.323751 | 0 | 0.225723048 | 2 | p-value_of_means_difference | 1.10E−29 |
| 169 | 14.885695 | 0 | 0.226723048 | 2 | n_group1 | 243 |
| 177 | 8.925394 | 1 | 0.208143048 | 2 | n_group2 | 52 |
| 202 | 3.378445 | 1 | 0.223143048 | 2 | Metasatasis | |
| | | | | | enrichment analysis | |
| 210 | 11.203285 | 0 | 0.282143048 | 2 | one-sided p-value | [symbol] |
| 212 | 12.145106 | 0 | 0.206143048 | 2 | (by Exact Ratio of Two | |
| | | | | | Binomial Proportions test) | |
| 214 | 7.477071 | 1 | 0.201143048 | 2 | 1. Binomial Proportions 1 | 0.321 |
| | | | | | (BP-1) no metastaes | |
| 215 | 10.351814 | 0 | 0.383143048 | 2 | 2. Binomial Proportions 2 | 0.442 |
| | | | | | (BP-2) metastaes | |
| 220 | 10.327173 | 0 | 0.247143048 | 2 | Ratio of Proportions: | [symbol] |
| | | | | | BP-2/BP-1 | |
| 221 | 10.376454 | 0 | 0.224143048 | 2 | Std. Error: (pooled | 0.073 |
| | | | | | estimate of stdev of | |
| | | | | | piHat_2-piHa [symbol] | |
| 222 | 2.253251 | 1 | 0.221143048 | 2 | | |
| 226 | 2.661191 | 0 | 0.363143048 | 2 | | |
| 227 | 3.356605 | 1 | 0.246143048 | 2 | | |
| 228 | 1.223819 | 1 | 0.264143048 | 2 | | |
| 229 | 1.61807 | 1 | 0.279143048 | 2 | | |
| 230 | 0.271047 | 1 | 0.216143048 | 2 | | |
| 231 | 3.581109 | 1 | 0.262143048 | 2 | | |
| 235 | 6.516085 | 0 | 0.203083048 | 2 | | |
| 236 | 2.483231 | 0 | 0.233083048 | 2 | | |
| 238 | 1.845311 | 1 | 0.242083048 | 2 | | |
| 239 | 8.093087 | 0 | 0.215083048 | 2 | | |
| 241 | 2.004107 | 1 | 0.194083048 | 2 | | |
| 249 | 5.316906 | 0 | 0.201083048 | 2 | | |
| 251 | 9.407255 | 0 | 0.202083048 | 2 | | |
| 270 | 2.962355 | 0 | 0.247083048 | 2 | | |
| 306 | 10.201232 | 0 | 0.282863048 | 2 | | |
| 307 | 1.965777 | 1 | 0.324863048 | 2 | | |
| 326 | 8.298426 | 0 | 0.279863048 | 2 | | |
| 328 | 5.577002 | 0 | 0.203863048 | 2 | | |
| 329 | 2.130048 | 0 | 0.206863048 | 2 | | |
| 338 | 6.3436 | 0 | 0.196863048 | 2 | | |
| 363 | 4.971937 | 1 | 0.197763048 | 2 | | |
| 371 | 1.968515 | 1 | 0.233763048 | 2 | | |
| 385 | 1.946612 | 1 | 0.299763048 | 2 | | |
| 387 | 8.213552 | 0 | 0.244763048 | 2 | | |
| 402 | 7.378508 | 0 | 0.492143048 | 2 | | |
| 103 | 4.952772 | 1 | 0.194143048 | 2 | | |
| 110 | 2.168378 | 1 | 0.206143048 | 2 | | |
| 113 | 0.996578 | 1 | 0.280143048 | 2 | | |
| 117 | 5.303217 | 0 | 0.217143048 | 2 | | |
| 120 | 10.097194 | 0 | 0.316143048 | 2 | | |
| 122 | 14.817248 | 0 | | 1 | | |
| 124 | 6.644764 | 0 | | 1 | | |
| 125 | 7.748118 | 0 | | 1 | | |
| 126 | 6.31896 | 1 | | 1 | | |
| 127 | 4.66256 | 1 | | 1 | | |
| 128 | 8.73922 | 0 | | 1 | | |
| 129 | 7.56742 | 0 | | 1 | | |
| 130 | 7.296372 | 0 | | 1 | | |
| 131 | 4.66256 | 0 | | 1 | | |
| 132 | 5.867214 | 0 | | 1 | | |
| 133 | 8.648871 | 0 | | 1 | | |
| 134 | 6.995209 | 1 | | 1 | | |
| 136 | 3.438741 | 1 | | 1 | | |
| 137 | 15.329227 | 0 | | 1 | | |
| 138 | 3.474333 | 1 | | 1 | | |
| 139 | 12.766598 | 0 | | 1 | | |
| 141 | 1.40178 | 1 | | 1 | | |
| 142 | 15.134839 | 0 | | 1 | | |
| 144 | 14.12731 | 0 | | 1 | | |
| 145 | 5.486653 | 0 | | 1 | | |
| 146 | 3.655031 | 1 | | 1 | | |
| 149 | 17.240246 | 0 | | 1 | | |
| 153 | 1.177276 | 1 | | 1 | | |
| 154 | 15.104723 | 0 | | 1 | | |
| 155 | 0.930869 | 1 | | 1 | | |
| 156 | 17.659138 | 0 | | 1 | | |
| 157 | 7.874059 | 0 | | 1 | | |
| 158 | 2.811773 | 1 | | 1 | | |
| 160 | 16.147844 | 0 | | 1 | | |
| 161 | 8.128679 | 1 | | 1 | | |
| 162 | 15.312799 | 0 | | 1 | | |
| 165 | 10.442163 | 1 | | 1 | | |
| 170 | 13.34976 | 0 | | 1 | | |
| 172 | 1.38809 | 1 | | 1 | | |
| 174 | 13.749487 | 0 | | 1 | | |
| 175 | 7.594798 | 1 | | 1 | | |
| 176 | 12.572211 | 0 | | 1 | | |
| 178 | 13.174538 | 0 | | 1 | | |
| 179 | 12.76386 | 0 | | 1 | | |
| 180 | 2.614648 | 1 | | 1 | | |
| 181 | 10.817248 | 0 | | 1 | | |
| 182 | 11.318275 | 0 | | 1 | | |
| 183 | 11.86037 | 0 | | 1 | | |
| 184 | 1.21013 | 1 | | 1 | | |
| 185 | 7.334702 | 0 | | 1 | | |
| 186 | 11.739904 | 0 | | 1 | | |
| 187 | 12.503765 | 0 | | 1 | | |
| 188 | 11.263518 | 0 | | 1 | | |
| 189 | 12.073922 | 0 | | 1 | | |
| 190 | 7.449692 | 0 | | 1 | | |
| 191 | 12.736482 | 0 | | 1 | | |
| 192 | 2.696783 | 1 | | 1 | | |
| 193 | 11.832991 | 0 | | 1 | | |
| 194 | 12.465435 | 1 | | 1 | | |
| 195 | 2.05065 | 0 | | 1 | | |
| 196 | 11.195072 | 0 | | 1 | | |
| 197 | 11.047228 | 0 | | 1 | | |
| 198 | 9.338809 | 0 | | 1 | | |
| 199 | 10.907598 | 0 | | 1 | | |
| 200 | 10.767967 | 0 | | 1 | | |
| 201 | 11.200548 | 0 | | 1 | | |
| 203 | 11.036277 | 0 | | 1 | | |
| 205 | 10.138261 | 0 | | 1 | | |
| 207 | 9.653662 | 0 | | 1 | | |
| 208 | 10.67488 | 0 | | 1 | | |
| 209 | 6.565366 | 1 | | 1 | | |
| 213 | 1.97399 | 1 | | 1 | | |
| 217 | 1.716632 | 1 | | 1 | | |
| 218 | 2.340862 | 1 | | 1 | | |
| 219 | 9.831622 | 0 | | 1 | | |
| 224 | 10.020534 | 0 | | 1 | | |
| 233 | 14.121834 | 0 | | 1 | | |
| 237 | 1.152635 | 1 | | 1 | | |
| 240 | 4.095825 | 1 | | 1 | | |
| 243 | 9.982204 | 0 | | 1 | | |
| 245 | 11.545517 | 0 | | 1 | | |
| 246 | 4.17796 | 0 | | 1 | | |
| 247 | 5.637235 | 0 | | 1 | | |
| 248 | 4.933607 | 0 | | 1 | | |
| 250 | 5.568789 | 0 | | 1 | | |
| 252 | 9.122519 | 1 | | 1 | | |
| 254 | 4.588638 | 1 | | 1 | | |
| 256 | 8.988364 | 1 | | 1 | | |
| 257 | 2.297057 | 1 | | 1 | | |
| 258 | 5.117043 | 1 | | 1 | | |
| 259 | 5.516769 | 1 | | 1 | | |
| 260 | 8.303901 | 1 | | 1 | | |
| 261 | 8.594114 | 0 | | 1 | | |
| 263 | 2.223135 | 1 | | 1 | | |
| 264 | 7.252567 | 0 | | 1 | | |
| 265 | 6.78987 | 0 | | 1 | | |
| 266 | 7.011636 | 0 | | 1 | | |
| 267 | 6.9295 | 0 | | 1 | | |
| 268 | 7.088296 | 0 | | 1 | | |
| 269 | 0.936328 | 1 | | 1 | | |
| 271 | 7.022587 | 0 | | 1 | | |
| 272 | 7.252567 | 0 | | 1 | | |
| 273 | 6.997947 | 0 | | 1 | | |
| 274 | 5.924709 | 0 | | 1 | | |
| 275 | 0.054757 | 0 | | 1 | | |
| 276 | 0.648871 | 1 | | 1 | | |
| 277 | 5.114305 | 0 | | 1 | | |
| 278 | 5.311431 | 0 | | 1 | | |
| 280 | 0.024641 | 0 | | 1 | | |
| 281 | 7.340178 | 0 | | 1 | | |
| 282 | 5.744011 | 0 | | 1 | | |
| 283 | 5.32512 | 1 | | 1 | | |
| 284 | 3.915127 | 0 | | 1 | | |
| 285 | 5.771389 | 0 | | 1 | | |
| 286 | 4.944559 | 0 | | 1 | | |
| 287 | 6.067077 | 1 | | 1 | | |
| 288 | 0.353183 | 0 | | 1 | | |
| 290 | 4.971937 | 0 | | 1 | | |
| 291 | 11.652293 | 0 | | 1 | | |
| 292 | 8.366872 | 0 | | 1 | | |
| 293 | 6.313484 | 0 | | 1 | | |
| 294 | 6.143737 | 0 | | 1 | | |
| 295 | 5.555099 | 0 | | 1 | | |
| 296 | 0.325804 | 0 | | 1 | | |
| 297 | 9.596167 | 0 | | 1 | | |
| 298 | 9.456537 | 0 | | 1 | | |
| 300 | 2.852841 | 1 | | 1 | | |
| 301 | 9.330595 | 0 | | 1 | | |
| 302 | 1.782341 | 0 | | 1 | | |
| 303 | 9.193703 | 0 | | 1 | | |
| 304 | 6.710472 | 1 | | 1 | | |
| 305 | 9.549624 | 0 | | 1 | | |
| 308 | 9.322382 | 0 | | 1 | | |
| 309 | 8.561259 | 1 | | 1 | | |
| 310 | 2.655715 | 0 | | 1 | | |
| 311 | 4.219028 | 1 | | 1 | | |
| 312 | 9.103354 | 0 | | 1 | | |
| 313 | 6.056126 | 1 | | 1 | | |
| 314 | 3.219713 | 1 | | 1 | | |
| 315 | 8.240931 | 0 | | 1 | | |
| 317 | 2.138261 | 1 | | 1 | | |
| 318 | 2.335387 | 1 | | 1 | | |
| 319 | 6.370979 | 1 | | 1 | | |
| 320 | 9.894593 | 0 | | 1 | | |
| 321 | 1.500342 | 1 | | 1 | | |
| 322 | 6.704997 | 0 | | 1 | | |
| 323 | 8.80219 | 0 | | 1 | | |
| 324 | 8.859685 | 0 | | 1 | | |
| 325 | 8.854209 | 0 | | 1 | | |
| 327 | 4.621492 | 1 | | 1 | | |
| 330 | 5.199179 | 0 | | 1 | | |
| 331 | 2.157426 | 1 | | 1 | | |
| 332 | 7.991786 | 0 | | 1 | | |
| 333 | 8.495551 | 0 | | 1 | | |
| 334 | 7.693361 | 0 | | 1 | | |
| 335 | 7.477071 | 0 | | 1 | | |
| 336 | 7.408624 | 0 | | 1 | | |
| 337 | 2.086242 | 0 | | 1 | | |
| 339 | 16.591376 | 0 | | 1 | | |
| 340 | 3.12115 | 1 | | 1 | | |
| 341 | 1.73306 | 1 | | 1 | | |
| 342 | 15.351129 | 0 | | 1 | | |
| 343 | 6.609172 | 0 | | 1 | | |
| 344 | 6.874743 | 0 | | 1 | | |
| 345 | 6.995209 | 0 | | 1 | | |
| 346 | 7.12115 | 0 | | 1 | | |
| 347 | 4.720055 | 0 | | 1 | | |
| 348 | 6.171116 | 0 | | 1 | | |
| 349 | 6.464066 | 0 | | 1 | | |
| 350 | 3.285421 | 0 | | 1 | | |
| 351 | 6.527036 | 0 | | 1 | | |
| 352 | 5.809719 | 0 | | 1 | | |
| 353 | 4.843258 | 0 | | 1 | | |
| 354 | 6.160164 | 0 | | 1 | | |
| 355 | 6.045175 | 0 | | 1 | | |
| 356 | 6.214921 | 0 | | 1 | | |
| 357 | 5.823409 | 0 | | 1 | | |
| 358 | 6.239562 | 0 | | 1 | | |
| 359 | 6.017796 | 0 | | 1 | | |
| 360 | 5.549624 | 0 | | 1 | | |
| 361 | 5.347023 | 0 | | 1 | | |
| 362 | 5.259411 | 0 | | 1 | | |
| 364 | 18.080767 | 0 | | 1 | | |
| 365 | 17.486653 | 0 | | 1 | | |
| 366 | 17.152635 | 0 | | 1 | | |
| 367 | 0.572211 | 1 | | 1 | | |
| 368 | 9.568789 | 1 | | 1 | | |
| 369 | 3.258042 | 1 | | 1 | | |
| 370 | 9.998631 | 1 | | 1 | | |
| 373 | 7.772758 | 0 | | 1 | | |
| 374 | 2.680356 | 1 | | 1 | | |
| 375 | 17.420945 | 0 | | 1 | | |
| 377 | 8.528405 | 1 | | 1 | | |
| 378 | 13.919233 | 0 | | 1 | | |
| 379 | 13.864476 | 0 | | 1 | | |
| 380 | 12.73922 | 0 | | 1 | | |
| 381 | 7.772758 | 0 | | 1 | | |
| 383 | 11.08282 | 0 | | 1 | | |
| 388 | 7.225188 | 0 | | 1 | | |
| 389 | 3.419576 | 1 | | 1 | | |
| 390 | 6.803559 | 0 | | 1 | | |
| 391 | 3.509925 | 0 | | 1 | | |
| 392 | 6.171116 | 0 | | 1 | | |
| 393 | 5.574264 | 0 | | 1 | | |
| 394 | 5.708419 | 0 | | 1 | | |
| 395 | 11.211499 | 1 | | 1 | | |
| 396 | 10.231348 | 0 | | 1 | | |
| 397 | 4.766598 | 1 | | 1 | | |
| 398 | 8.424367 | 0 | | 1 | | |
| 401 | 1.527721 | 1 | | 1 | | |
| 403 | 6.754278 | 0 | | 1 | | |
| 404 | 7.570157 | 0 | | 1 | | |
| 107 | 2.543463 | 1 | | 1 | | |
| 109 | 3.195072 | 1 | | 1 | | |
| 111 | 1.270363 | 1 | | 1 | | |
| 118 | 5.232033 | 0 | | 1 | | |
| 4 | 12.996578 | 0 | | 1 | | |
| 6 | 11.156742 | 0 | | 1 | | |
| 7 | 10.138261 | 0 | | 1 | | |
| 8 | 8.80219 | 0 | | 1 | | |
| 9 | 10.294319 | 0 | | 1 | | |
| 11 | 5.804244 | 0 | | 1 | | |
| 12 | 7.857632 | 0 | | 1 | | |
| 13 | 8.167009 | 0 | | 1 | | |
| 14 | 8.232717 | 0 | | 1 | | |
| 17 | 7.865845 | 0 | | 1 | | |
| 26 | 6.970568 | 0 | | 1 | | |
| 27 | 5.185489 | 0 | | 1 | | |
| 28 | 6.245038 | 0 | | 1 | | |
| 29 | 11.389459 | 0 | | 1 | | |
| 36 | 10.108145 | 0 | | 1 | | |
| 38 | 7.353867 | 0 | | 1 | | |
| 39 | 11.017112 | 0 | | 1 | | |
| 45 | 1.089665 | 1 | | 1 | | |
| 48 | 1.026694 | 1 | | 1 | | |
| 51 | 4.906229 | 1 | | 1 | | |
| 56 | 4.695414 | 1 | | 1 | | |
| 57 | 2.297057 | 1 | | 1 | | |
| 58 | 1.122519 | 1 | | 1 | | |
| 59 | 4.629706 | 1 | | 1 | | |
| 60 | 4.892539 | 1 | | 1 | | |
| 61 | 2.680356 | 1 | | 1 | | |
| 62 | 0.807666 | 1 | | 1 | | |
| 71 | 1.982204 | 1 | | 1 | | |
| 72 | 3.028063 | 1 | | 1 | | |
| 73 | 2.149213 | 1 | | 1 | | |
| 75 | 2.209446 | 1 | | 1 | | |
| 76 | 2.12731 | 1 | | 1 | | |
| | | 78 | | 243 | | |

B. Metadata

| | | | X209027_s_at | ABI1 (1: no | | |
|---|---|---|---|---|---|---|
| | Metastasis | EVENT: | (log2 expression | metastases; 2: | | |
| Sample_id | TIME(year) | metastais | signal) | metasatases) | Gene | ABI1 |
| X146B39 | 0.67 | 1 | 8.1 | 2 | Probe Sets | 209027_s_at |
| X164B81 | 11.33 | 0 | 8.13 | 2 | Refseq | NM_005470 |
| X182B43 | 11.08 | 0 | 8.12 | 2 | EntrezID | 10006 |
| X187B36 | 0 | 1 | 8.06 | 2 | Description | abI-interactor 1 |
| X226C06 | 10.75 | 0 | 8.79 | 2 | K-M: p-value | [symbol] |
| | | | | | (Log-runk test) | |
| X256C45 | 1.25 | 1 | 8.05 | 2 | Rank | 4 |
| X269C68 | 0 | 0 | 8.05 | 2 | Weights | 0.105 |
| X279C61 | 10.17 | 0 | 8.2 | 2 | cutoff | 8.040 |
| X311A27 | 1.75 | 1 | 8.13 | 2 | survival model | [symbol] |
| | | | | | design | |
| X314B55 | 9.58 | 1 | 8.18 | 2 | mean_group1 | 7.42 |
| X50A91 | 9.08 | 1 | 8.11 | 2 | mean_group2 | 8.18 |
| X54A09 | 12.58 | 0 | 8.14 | 2 | p-value_of_means_difference | 1.31E−09 |
| X66A84 | 1.25 | 1 | 8.28 | 2 | n_group1 | 236 |
| X100B08 | 11.83 | 0 | 6.8 | 1 | n_group2 | 13 |
| X101B88 | 11.83 | 0 | 7.4 | 1 | Metasatasis | |
| | | | | | enrichment | |
| | | | | | analysis | |
| X102B06 | 11.83 | 0 | 7.05 | 1 | one-sided p-value | [symbol] |
| X103B41 | 11.75 | 0 | 7.26 | 1 | (by Exact Ratio of Two | |
| | | | | | Binomial Proportions test) | |
| X104B91 | 3.58 | 0 | 7.56 | 1 | 1. Binomial Proportions | 0.2754 |
| | | | | | 1 ( BP-1) no metastaes | |
| X105B13 | 11.75 | 0 | 7.15 | 1 | 2. Binomial Proportions | 0.5385 |
| | | | | | 2 ( BP-2) metastaes | |
| X106B55 | 6.92 | 1 | 7.14 | 1 | Ratio of Proportions: | 1.955 |
| | | | | | BP-2/BP-1 | |
| X10B88 | 11.33 | 0 | 7.87 | 1 | Std. Error: (pooled | 0.1292 |
| | | | | | estimate of stdev of | |
| | | | | | piHat_2-piHat_1) | |
| X110B34 | 11.67 | 0 | 8.02 | 1 | | |
| X111B51 | 0.5 | 1 | 7.66 | 1 | | |
| X112B55 | 0.92 | 1 | 6.91 | 1 | | |
| X113B11 | 3.08 | 1 | 7.23 | 1 | | |
| X114B68 | 11.17 | 0 | 6.96 | 1 | | |
| X11B47 | 7.42 | 0 | 7.54 | 1 | | |
| X120B73 | 11.58 | 0 | 7.21 | 1 | | |
| X122B81 | 11.17 | 0 | 7.58 | 1 | | |
| X124B25 | 5 | 1 | 7.63 | 1 | | |
| X127B00 | 1.92 | 1 | 7.3 | 1 | | |
| X128B48 | 4.58 | 0 | 7.64 | 1 | | |
| X130B92 | 4.42 | 1 | 7.21 | 1 | | |
| X131B79 | 1.75 | 1 | 7.73 | 1 | | |
| X134B33 | 2 | 1 | 7.33 | 1 | | |
| X135B40 | 9.5 | 1 | 7.41 | 1 | | |
| X136B04 | 11.5 | 0 | 7.54 | 1 | | |
| X137B88 | 10.5 | 1 | 7.76 | 1 | | |
| X138B34 | 11.5 | 0 | 7.47 | 1 | | |
| X139B03 | 0.33 | 1 | 7.52 | 1 | | |
| X13B79 | 10.83 | 0 | 7.69 | 1 | | |
| X140B91 | 11.5 | 0 | 7.32 | 1 | | |
| X142B05 | 11.5 | 0 | 7.06 | 1 | | |
| X143B81 | 2.17 | 0 | 7.23 | 1 | | |
| X144B49 | 11.5 | 0 | 7.57 | 1 | | |
| X145B10 | 11.42 | 0 | 7.9 | 1 | | |
| X147B19 | 5.08 | 0 | 7.17 | 1 | | |
| X14B98 | 10.83 | 0 | 7.59 | 1 | | |
| X150B81 | 11.42 | 0 | 6.51 | 1 | | |
| X151B84 | 11.42 | 0 | 6.98 | 1 | | |
| X152B99 | 2.08 | 0 | 7.41 | 1 | | |
| X153B09 | 11.42 | 0 | 7.58 | 1 | | |
| X154B42 | 3.42 | 1 | 7.59 | 1 | | |
| X155B52 | 11.42 | 0 | 7.34 | 1 | | |
| X156B01 | 11.42 | 0 | 7.22 | 1 | | |
| X158B84 | 8.5 | 0 | 7.68 | 1 | | |
| X159B47 | 6.5 | 1 | 7.23 | 1 | | |
| X15C94 | 4.42 | 0 | 7.63 | 1 | | |
| X160B16 | 5.5 | 0 | 7.23 | 1 | | |
| X161B31 | 11.42 | 0 | 7.12 | 1 | | |
| X162B98 | 8.92 | 1 | 7.1 | 1 | | |
| X163B27 | 11.33 | 0 | 7.2 | 1 | | |
| X165B72 | 1.5 | 1 | 7.61 | 1 | | |
| X166B79 | 11.33 | 0 | 7.43 | 1 | | |
| X168B51 | 5.33 | 0 | 7.77 | 1 | | |
| X169B79 | 11.33 | 0 | 7.91 | 1 | | |
| X16C97 | 3.58 | 1 | 7.68 | 1 | | |
| X170B15 | 4.08 | 1 | 7.76 | 1 | | |
| X171B77 | 7 | 1 | 7.22 | 1 | | |
| X172B19 | 11.25 | 0 | 7.67 | 1 | | |
| X173B43 | 11.25 | 0 | 7.51 | 1 | | |
| X174B41 | 3.33 | 1 | 7.07 | 1 | | |
| X175B72 | 0 | 1 | 7.56 | 1 | | |
| X176B74 | 6 | 0 | 7.22 | 1 | | |
| X177B67 | 6.83 | 1 | 7.62 | 1 | | |
| X178B74 | 7.42 | 0 | 7.67 | 1 | | |
| X179B28 | 2.33 | 1 | 7.56 | 1 | | |
| X17C40 | 1.92 | 0 | 7.73 | 1 | | |
| X180B38 | 7.25 | 0 | 7.65 | 1 | | |
| X181B70 | 11.08 | 0 | 7.68 | 1 | | |
| X183B75 | 7 | 1 | 7.55 | 1 | | |
| X184B38 | 11 | 0 | 6.69 | 1 | | |
| X185B44 | 11 | 0 | 7.33 | 1 | | |
| X186B22 | 0.17 | 1 | 7.71 | 1 | | |
| X188B13 | 11 | 0 | 7.51 | 1 | | |
| X189B83 | 11 | 0 | 7.89 | 1 | | |
| X18C56 | 10.75 | 0 | 7.26 | 1 | | |
| X191B79 | 11 | 0 | 7.65 | 1 | | |
| X192B69 | 7.92 | 0 | 6.23 | 1 | | |
| X193B72 | 10.92 | 0 | 7.85 | 1 | | |
| X194B60 | 3.58 | 0 | 7.48 | 1 | | |
| X195B75 | 3 | 0 | 7.48 | 1 | | |
| X196B81 | 10.92 | 0 | 7.25 | 1 | | |
| X197B95 | 10.92 | 0 | 7.53 | 1 | | |
| X198B90 | 10.92 | 0 | 7.47 | 1 | | |
| X199B55 | 10.92 | 0 | 7.98 | 1 | | |
| X19C33 | 0 | 1 | 7.36 | 1 | | |
| X200B47 | 10.92 | 0 | 7.37 | 1 | | |
| X201B68 | 10.92 | 0 | 7.81 | 1 | | |
| X202B44 | 10.83 | 0 | 7.8 | 1 | | |
| X203B49 | 10.83 | 0 | 7.08 | 1 | | |
| X204B85 | 10.58 | 0 | 7.66 | 1 | | |
| X205B99 | 4.42 | 0 | 7.43 | 1 | | |
| X206C05 | 6.42 | 0 | 7.43 | 1 | | |
| X207008 | 10.83 | 0 | 7.41 | 1 | | |
| X208C06 | 0.08 | 0 | 7.45 | 1 | | |
| X209C10 | 10.83 | 0 | 7.42 | 1 | | |
| X210C72 | 10.83 | 0 | 7.84 | 1 | | |
| X211C88 | 1.5 | 0 | 7.69 | 1 | | |
| X212C21 | 10.83 | 0 | 7.78 | 1 | | |
| X213C36 | 10.08 | 0 | 7.36 | 1 | | |
| X216C61 | 10.75 | 0 | 7.64 | 1 | | |
| X217C79 | 10.75 | 0 | 7.6 | 1 | | |
| X218C29 | 10.75 | 0 | 7.64 | 1 | | |
| X21C28 | 10.67 | 0 | 7.31 | 1 | | |
| X220C70 | 10.75 | 0 | 7.39 | 1 | | |
| X221C14 | 10.67 | 0 | 7.03 | 1 | | |
| X222C26 | 0.08 | 1 | 7.11 | 1 | | |
| X223C51 | 9.92 | 1 | 7.71 | 1 | | |
| X224C93 | 7.92 | 0 | 7.03 | 1 | | |
| X225C52 | 10.75 | 0 | 7.83 | 1 | | |
| X227C50 | 10.75 | 0 | 7.2 | 1 | | |
| X229044 | 10.67 | 0 | 7.15 | 1 | | |
| X22C62 | 4.83 | 0 | 7.68 | 1 | | |
| X230047 | 0.5 | 0 | 7.73 | 1 | | |
| X231C80 | 6.42 | 0 | 7.57 | 1 | | |
| X232C58 | 1.75 | 0 | 7.92 | 1 | | |
| X233C91 | 10.67 | 0 | 7.35 | 1 | | |
| X234C15 | 10.67 | 0 | 7.48 | 1 | | |
| X235C20 | 10.67 | 0 | 7.77 | 1 | | |
| X236C55 | 10.67 | 0 | 7.55 | 1 | | |
| X237C56 | 10.67 | 0 | 7.56 | 1 | | |
| X238C87 | 10.67 | 0 | 7.38 | 1 | | |
| X23C52 | 8.5 | 1 | 7.66 | 1 | | |
| X240C54 | 2.4 | 1 | 7.79 | 1 | | |
| X241C01 | 6.75 | 1 | 7.27 | 1 | | |
| X242C21 | 10.58 | 0 | 7.3 | 1 | | |
| X243C70 | 10.58 | 0 | 7.72 | 1 | | |
| X244C89 | 7.25 | 1 | 7.64 | 1 | | |
| X245C22 | 0 | 1 | 7.07 | 1 | | |
| X246C75 | 10.17 | 0 | 7.42 | 1 | | |
| X247C76 | 10.5 | 0 | 7.86 | 1 | | |
| X248C91 | 10.5 | 0 | 7.51 | 1 | | |
| X249C42 | 0.17 | 0 | 7.62 | 1 | | |
| X24C30 | 10.67 | 0 | 7.14 | 1 | | |
| X250C78 | 2.5 | 0 | 7.76 | 1 | | |
| X251C14 | 10.5 | 0 | 7.33 | 1 | | |
| X252C64 | 3.08 | 1 | 7.47 | 1 | | |
| X253C20 | 8 | 0 | 7.39 | 1 | | |
| X254C80 | 10.5 | 0 | 6.81 | 1 | | |
| X255006 | 10.5 | 0 | 7.51 | 1 | | |
| X257C87 | 10.5 | 0 | 7.7 | 1 | | |
| X258C21 | 10.33 | 0 | 7.25 | 1 | | |
| X259C74 | 10.42 | 0 | 7.3 | 1 | | |
| X260C91 | 10.42 | 0 | 7.87 | 1 | | |
| X261C94 | 10.42 | 0 | 6.92 | 1 | | |
| X262C85 | 10.42 | 0 | 7.63 | 1 | | |
| X263C82 | 8.08 | 1 | 7.26 | 1 | | |
| X265C40 | 10.42 | 0 | 7.21 | 1 | | |
| X266C51 | 10.42 | 0 | 6.97 | 1 | | |
| X267C04 | 10.33 | 0 | 7.61 | 1 | | |
| X268C87 | 10.33 | 0 | 7.77 | 1 | | |
| X26C23 | 1.17 | 1 | 7.81 | 1 | | |
| X270C93 | 10.33 | 0 | 7.28 | 1 | | |
| X271C71 | 1.17 | 1 | 7.62 | 1 | | |
| X272C88 | 10.33 | 0 | 7.24 | 1 | | |
| X274C81 | 10.33 | 0 | 7.14 | 1 | | |
| X275C70 | 10.25 | 0 | 7.34 | 1 | | |
| X277C64 | 8.58 | 0 | 7.33 | 1 | | |
| X278C80 | 10.25 | 0 | 7.25 | 1 | | |
| X27C82 | 6.8 | 0 | 7.6 | 1 | | |
| X280C43 | 10.08 | 0 | 7.54 | 1 | | |
| X282C51 | 9.92 | 0 | 7.3 | 1 | | |
| X284C63 | 10.08 | 0 | 7.57 | 1 | | |
| X286C91 | 10 | 0 | 7.35 | 1 | | |
| X287C67 | 10 | 0 | 7.25 | 1 | | |
| X288C57 | 10 | 0 | 6.61 | 1 | | |
| X289C75 | 10 | 0 | 7.12 | 1 | | |
| X28C76 | 10.5 | 0 | 7.58 | 1 | | |
| X290C91 | 10 | 0 | 7.31 | 1 | | |
| X291C17 | 0.92 | 0 | 7.05 | 1 | | |
| X292C66 | 10 | 0 | 7.61 | 1 | | |
| X294004 | 10 | 0 | 7.74 | 1 | | |
| X296C95 | 9.92 | 0 | 7.62 | 1 | | |
| X297C26 | 9.92 | 0 | 7.04 | 1 | | |
| X298C47 | 6.5 | 1 | 6.9 | 1 | | |
| X301C66 | 9.92 | 0 | 7.61 | 1 | | |
| X303C36 | 9.92 | 0 | 7.08 | 1 | | |
| X304C89 | 2.58 | 0 | 7.16 | 1 | | |
| X307C50 | 9.83 | 0 | 7.51 | 1 | | |
| X308C93 | 2.25 | 0 | 7.13 | 1 | | |
| X309049 | 9.83 | 0 | 7.67 | 1 | | |
| X313A87 | 10.17 | 0 | 7.61 | 1 | | |
| X316C65 | 10.75 | 0 | 7.23 | 1 | | |
| X33C30 | 10.17 | 0 | 7.22 | 1 | | |
| X34C80 | 10.17 | 0 | 7.45 | 1 | | |
| X35C29 | 6.08 | 0 | 7.88 | 1 | | |
| X36C17 | 10.08 | 0 | 7.38 | 1 | | |
| X37C06 | 10 | 0 | 7.28 | 1 | | |
| X39C24 | 10 | 0 | 7.3 | 1 | | |
| X40C57 | 10 | 0 | 7.39 | 1 | | |
| X41C65 | 9.92 | 0 | 7.46 | 1 | | |
| X42C57 | 9.92 | 0 | 7.1 | 1 | | |
| X43047 | 9.92 | 0 | 7.28 | 1 | | |
| X44A53 | 12.75 | 0 | 7.52 | 1 | | |
| X45A96 | 12.75 | 0 | 7.7 | 1 | | |
| X46A25 | 0 | 1 | 7.32 | 1 | | |
| X47A87 | 10.58 | 1 | 7.74 | 1 | | |
| X48A46 | 1.83 | 0 | 7.11 | 1 | | |
| X49A07 | 6.67 | 1 | 7.87 | 1 | | |
| X51A98 | 12.67 | 0 | 6.93 | 1 | | |
| X52A90 | 12.67 | 0 | 7.47 | 1 | | |
| X53A06 | 3.17 | 1 | 7.7 | 1 | | |
| X55A79 | 3.25 | 0 | 7.06 | 1 | | |
| X56A94 | 1.08 | 1 | 6.66 | 1 | | |
| X58A50 | 1.33 | 1 | 7.08 | 1 | | |
| X5B97 | 5.5 | 1 | 7.45 | 1 | | |
| X60A05 | 0.67 | 1 | 6.61 | 1 | | |
| X61A53 | 12.5 | 0 | 7.06 | 1 | | |
| X62A02 | 0.25 | 1 | 7.3 | 1 | | |
| X63A62 | 0.17 | 1 | 7.07 | 1 | | |
| X64A59 | 12.42 | 0 | 7.38 | 1 | | |
| X65A68 | 12.42 | 0 | 7.47 | 1 | | |
| X67A43 | 0.92 | 1 | 7.33 | 1 | | |
| X69A93 | 12.42 | 0 | 7.86 | 1 | | |
| X6B85 | 0.58 | 1 | 7.45 | 1 | | |
| X70A79 | 12 | 0 | 8.04 | 1 | | |
| X72A92 | 12.33 | 0 | 7.76 | 1 | | |
| X73A01 | 12.33 | 0 | 7.78 | 1 | | |
| X74A63 | 5.92 | 1 | 7.17 | 1 | | |
| X75A01 | 11.92 | 1 | 7.27 | 1 | | |
| X76A44 | 0 | 1 | 6.9 | 1 | | |
| X77A50 | 1.08 | 1 | 7.52 | 1 | | |
| X79A35 | 0.83 | 0 | 7.55 | 1 | | |
| X7B96 | 2.42 | 1 | 7.33 | 1 | | |
| X82A83 | 2.58 | 0 | 7.25 | 1 | | |
| X83A37 | 12.17 | 0 | 7.1 | 1 | | |
| X84A44 | 12.17 | 0 | 7.72 | 1 | | |
| X85A03 | 2.08 | 0 | 7.47 | 1 | | |
| X86A40 | 12.17 | 0 | 7.82 | 1 | | |
| X87A79 | 12.08 | 0 | 7.63 | 1 | | |
| X88A67 | 12.08 | 0 | 7.62 | 1 | | |
| X89A64 | 12.08 | 0 | 7.95 | 1 | | |
| X8B87 | 11.33 | 0 | 6.95 | 1 | | |
| X90A63 | 2.67 | 0 | 7.61 | 1 | | |
| X94A16 | 11.08 | 1 | 7.3 | 1 | | |
| X96A21 | 0.08 | 1 | 7.15 | 1 | | |
| X99A50 | 10.5 | 0 | 6.93 | 1 | | |
| X9B52 | 11.33 | 0 | 7.61 | 1 | | |

[symbol]

indicates data missing or illegible when filed

TABLE 5
Survival significant prognostic genes encoding WAVE
complex and NDEL gene are correlated with expression
of ABI1. METABRIC breast cancer Dataset (n = 1904)

| Correlated | Spearman | |
|---|---|---|
| Gene | Correlation | p-value |
| BRK1 | 0.0802 | 4.61E−04 |
| CYFIP1 | 0.114 | 5.09E−07 |
| CYFIP2 | −0.144 | 2.04E−10 |
| WASF3 | 0.115 | 5.87E−07 |
| NDEL1 | 0.0777 | 6.90E−04 |
| RAC1 | −0.036 | 0.133 |

TABLE 6
Significance of association between the survival stratification
grouping (by DMFS) and metastatic events (A, B) and our estimations
of the probability of metastasis risk events(C, D)
A
Rosetta cohort (NCI cohort van de Vijver al, 2002)

| Group, DMFS | all 7 probes, 3 groups |
|---|---|
| SWVg Score(log-rank p-value) | 8.78E−12 |
| coxph_g1_vs_g2 | 0.003377107 |
| coxph_g1_vs_g3 | 2.30E−10 |
| coxph_g2_vs_g3 | 2.58E−08 |
| n_group1 | 86 |
| n_group2 | 171 |
| n_group3 | 38 |
| Kruskal-Wallis Test | |
| 2-sided p-value | 7.02E−11 |

B
Metadata cohort (Ivshina et al, 2006; Miller et al, 2005)

| Group, DMFS | all 7 probes, 3 groups |
|---|---|
| SWVg Score(log-rank p-value) | 4.49E−11 |
| coxph_g1_vs_g2 | 6.48E−07 |
| coxph_g1_vs_g3 | 9.44E−08 |
| coxph_g2_vs_g3 | 0.0287 |
| n_group1 | 135 |
| n_group2 | 101 |
| n_group3 | 13 |
| Kruskal-Wallis Test | |
| 2-sided p-value | 2.772E−08 |

C

| K-M survival function | Low | Intermediate | High | Total |
|---|---|---|---|---|
| group 1 | 1 | 2 | 3 | |
| Meta+ | 13 | 58 | 30 | 101 |
| Meta− | 73 | 113 | 8 | 194 |
| Mets, total | 86 | 171 | 38 | 295 |
| Probability of | 0.15 | 0.34 | 0.79 | |
| Metastasis risk event | | | | |
| Median, year | 7.57 | 6.52 | 3.33 | |
| Mean, year | 7.97 | 7.15 | 4.35 | |
| Confidence −95.000% | 3.289 | 6.506 | 7.126 | |
| Confidence +95.000% | 5.409 | 7.798 | 8.809 | |

D

| K-M survival function | Low | Intermediate | High | Total |
|---|---|---|---|---|
| group 1 | 1 | 2 | 3 | |
| Meta+ | 20 | 44 | 8 | 72 |
| Meta− | 115 | 57 | 5 | 177 |
| Mets, total | 135 | 101 | 13 | 249 |
| Probability of | 0.15 | 0.44 | 0.62 | |
| Metastasis risk event | | | | |
| Median, year | 1.25 | 7.92 | 10.50 | |
| Mean, year | 3.364615 | 6.709208 | 9.287630 | |
| Confidence −95.000% | 0.842884 | 5.844983 | 8.746575 | |
| Confidence +95.000% | 5.886347 | 7.573433 | 9.828685 | |

TABLE 7
Abi1-based signature predicts distant metastatic events more accurately
than commonly used clinical factors. Cox univariate and multivariate
hazards proportional models analysis compars the ABI1-based 7-gene
metastasis risk classifier (low, moderate, high risks by SVWg),
ESR status (ER (+) , ER(−)), and lymph node status (LN(+),
LN(−)) to predict metastatic events in the Rosetta cohort.

| Univariate model | beta | HR (95% CI for HR) | | p-value |
|---|---|---|---|---|
| Lymph nodal status (0, 1) | −0.12 | 0.89 | (0.6-1.3) | 0.55 |
| ESR1 status (1, 0) | −0.65 | 0.52 | (0.34-0.8) | 0.0026 |
| ABI1-based 7-gene | 0.96 | 2.6 | (1.9-3.4) | 7.02E−11 |
| expression signature | | | | |
| metastasis risk | | | | |
| classifier (3 groups) | | | | |

| Multivariate model | | | | |
|---|---|---|---|---|
| (3 groups) | beta | HR (95% CI for HR) | | p-value |
| Lymph nodal status (0, 1) | −0.086 | 0.92 | (0.62-1.4) | 0.67 |
| ESR1 status (1, 0) | −0.63 | 0.53 | (0.35-0.82) | 0.004 |
| ABI1-based 7-gene | 0.92 | 2.4 | (1.7-3.5) | 2.20E−10 |
| expression signature | | | | |
| metastsis risk | | | | |
| classifier (1, 2, 3) | | | | |

TABLE 8
Basal-like vs. luminal cell type markers in primary breast tumors of Abi1 KO mice

| | Mouse ID | G144 | G164 | G184 | G174 | | | | | Breast |
|---|---|---|---|---|---|---|---|---|---|---|
| | Genotype | fl/wt | fl/wt | fl/fl | fl/fl | Fold Change Depletion | | | Treatment | cancer |
| | Cre | − | + | − | + | Heterozygous | Homozygous | Ratio | Effect* | subtype |
| Gene | Krt14 | 2425 | 7762 | 9447 | 3127 | 3.2 | 0.33 | 9.67 | yes | basal |
| expression | Vim | 23206 | 34115 | 49031 | 30226 | 1.47 | 0.62 | 2.38 | yes | basal |
| (RNA-seq | Krt8 | 96334 | 82757 | 104627 | 87764 | 0.86 | 0.84 | 1.02 | no | Luminal |
| from | Krt18 | 102915 | 93062 | 110645 | 99089 | 0.9 | 0.89 | 1.01 | no | Luminal |
| primary | Sox9 | 9053 | 9355 | 12069 | 11021 | 1.03 | 0.91 | 1.13 | no | Luminal |
| tumors) | | | | | | | | | | progenitor |
| | Estr1 | 3501 | 3685 | 5312 | 3067 | 1.05 | 0.58 | 1.82 | no | Luminal |

TABLE 9
Mouse breast pathology Ki67 and grading data

| | | | | | | | | | |
|---|---|---|---|---|---|---|---|---|---|
| | | | | | | | | Presence of | |
| Healthy | Mouse | Tissue | | Age of | Ki-67, | | Grade of worst | satellite | |
| controls | number | type | Genotype | mouse | % | Ki-67 | breast lesion* | nodules | Comments |

Healthy controls <20 weeks

| | | | | | | | | | |
|---|---|---|---|---|---|---|---|---|---|
| 42 | G279-4 | Breast | non tumor | 17 | 6 | no tumor | N/A | N/A | Normal breast, |
| | | | control | | | | | | intraparenchymal |
| | | | | | | | | | LN |
| 43 | G286-4 | Breast | non tumor | 17 | 6 | no tumor | N/A | N/A | Normal breast, |
| | | | control | | | | | | intraparenchymal |
| | | | | | | | | | LN |
| 44 | G293-4 | Breast | non tumor | 17.14 | 14 | no tumor | N/A | N/A | Normal breast, |
| | | | control | | | | | | intraparenchymal |
| | | | | | | | | | LN |
| 45 | G294-4 | Breast | non tumor | 17.14 | 16 | no tumor | N/A | N/A | Normal breast, |
| | | | control | | | | | | intraparenchymal |
| | | | | | | | | | LN |
| 46 | G300-4 | Breast | non tumor | 17.14 | 7 | no tumor | N/A | N/A | Normal breast, |
| | | | control | | | | | | intraparenchymal |
| | | | | | | | | | LN |
| 47 | G301-4 | Breast | non tumor | 17.14 | 3 | no tumor | N/A | N/A | Normal breast, |
| | | | control | | | | | | intraparenchymal LN |
| 48 | G303-4 | Breast | non tumor | 17.14 | 7 | no tumor | N/A | N/A | Normal breast |
| | | | control | | | | | | parenchyma |

Healthy controls >20 weeks

| | | | | | | | | | |
|---|---|---|---|---|---|---|---|---|---|
| 56 | G203-4 | Breast | healthy | 21.14 | 15 | no tumor | N/A | N/A | Normal breast, |
| | | | control | | | | | | intraparenchymal |
| | | | | | | | | | LN |
| 62 | G217-4 | Breast | non tumor | 21 | | | N/A | N/A | Normal breast, |
| | | | control | | | | | | intraparenchymal |
| | | | | | | | | | LN |
| 66 | G221-4 | Breast | non tumor | 21.3 | 15 | no tumor | N/A | N/A | Normal breast, |
| | | | control | | | | | | intraparenchymal |
| | | | | | | | | | LN |
| 67 | G238-4 | Breast | non tumor | 21 | 6 | no tumor | N/A | N/A | Normal breast, |
| | | | control | | | | | | intraparenchymal |
| | | | | | | | | | LN |
| 68 | IG245-4 | Breast | non tumor | 21 | 6 | no tumor | N/A | N/A | Normal breast, |
| | | | control | | | | | | parenchyma |
| 69 | G262-4 | Breast | non tumor | 21 | 9 | no tumor | N/A | N/A | Normal breast |
| | | | control | | | | | | parenchyma, |
| | | | | | | | | | intraparenchymal |
| | | | | | | | | | LN |
| 70 | G270-4 | Breast | non tumor | 21.3 | 3 | no tumor | N/A | N/A | Normal breast |
| | | | control | | | | | | parenchyma, |
| | | | | | | | | | intraparenchymal |
| | | | | | | | | | LN |
| 71 | G274-4 | Breast | non tumor | 21.3 | 11 | no tumor | N/A | N/A | Normal breast |
| | | | control | | | | | | parenchyma, |
| | | | | | | | | | intraparenchymal |
| | | | | | | | | | LN |
| 74 | G194-4 | Breast | non tumor | 26.14 | 8 | no tumor | N/A | N/A | Normal breast |
| | | | control | | | | | | parenchyma |

Homozygous controls <20 weeks

| | | | | | | | | Presence of | |
|---|---|---|---|---|---|---|---|---|---|
| Homozygous | Mouse | Tissue | | Age of | Ki-67, | | Grade of worst | satellite | |
| controls | number | type | Genotype | mouse | % | | breast lesion* | nodules | Comments |
| 32 | G281-9 | Breast | hom c | 17.3 | 24 | | 3 | Yes | Cribiform and |
| | | | ontrol | | | | | | solid growth |
| | | | | | | | | | patterns, |
| | | | | | | | | | apoptotic |
| | | | | | | | | | tumor cells, |
| | | | | | | | | | but no large |
| | | | | | | | | | areas of |
| | | | | | | | | | necrosis |
| 33 | G284-6 | Breast | hom | 17 | 51 | | 4 | No, but | Sheet like |
| | | | control | | | | | continuous | growth, |
| | | | | | | | | tumor | mitotically |
| | | | | | | | | | active with |
| | | | | | | | | | atypical |
| | | | | | | | | | mitoses, |
| | | | | | | | | | many single |
| | | | | | | | | | apoptic tumor |
| | | | | | | | | | cells but no |
| | | | | | | | | | confluent |
| | | | | | | | | | areas of |
| | | | | | | | | | necrosis |
| 34 | G285-9 | Breast | hom | 17 | 47 | in DCIS | 2 | N/A all | High grade |
| | | | control | | | area | | in situ | MIN/DCIS |
| | | | | | | | | | with |
| | | | | | | | | | cribiform |
| | | | | | | | | | and solid |
| | | | | | | | | | patterns, |
| | | | | | | | | | single |
| | | | | | | | | | apoptotic |
| | | | | | | | | | tumor cells |
| | | | | | | | | | without large |
| | | | | | | | | | areas of |
| | | | | | | | | | comedonecrosis |
| 99 | G335-4 | Breast | hom | 17 | 83 | | 2 | N/A all | DCIS, |
| | | | control | | | | | in situ | intermediate |
| | | | | | | | | | nuclear grade. |
| | | | | | | | | | Also an |
| | | | | | | | | | intraparenchymal |
| | | | | | | | | | LN |

| | | | | | | | | | |
|---|---|---|---|---|---|---|---|---|---|
| | | | | | | | | Presence of | |
| Homozygous | Mouse | Tissue | | Age of | Ki-67, | | Grade of worst | satellite | |
| controls | number | type | Genotype | mouse | % | Ki-67 | breast lesion* | nodules | Comments |

Homozygous controls >20 weeks

| | | | | | | | | |
|---|---|---|---|---|---|---|---|---|
| 7 | G113-6 | Breast | hom | 21.6 | 68 | 4 | Present | Abundant |
| | | | control | | | | | apoptotic |
| | | | | | | | | tumor cells |
| 15 | G184-8 | Breast | hom | 22.9 | 39 | 4 | Present | Majority of |
| | | | control | | | | | tumor with |
| | | | | | | | | solid growth, |
| | | | | | | | | comedonecrosis, |
| | | | | | | | | and abundant |
| | | | | | | | | apoptotic |
| | | | | | | | | tumor cells |
| 16 | G113-4 | Breast | hom | 24.7 | 47 | 4 | Present | Majority of |
| | | | control | | | | | tumor with |
| | | | | | | | | solid growth, |
| | | | | | | | | comedonecrosis, |
| | | | | | | | | and abundant |
| | | | | | | | | apoptotic |
| | | | | | | | | tumor cells |
| 17 | G111-8 | Breast | hom | 24.7 | 32 | 3 | 0 | Well- |
| | | | control | | | | | differentiated, |
| | | | | | | | | tubular growth |
| 18 | G111-1 | Breast | hom | 24.7 | 61 | 4 | Present | Poorly |
| | | | control | | | | | differentiated, |
| | | | | | | | | comedo and |
| | | | | | | | | infarct |
| | | | | | | | | like necrosis |
| 20 | G208-8 | Breast | hom | 21.14 | | 4 | No | Squamous |
| | | | control | | | | | differentiated |
| | | | | | | | | of de- |
| | | | | | | | | differentiated |
| | | | | | | | | tumor |
| 28 | G246-8 | Breast | hom | 21 | | 4 | Yes | Squamous |
| | | | control | | | | | differentiated |
| | | | | | | | | of de- |
| | | | | | | | | differentiated |
| | | | | | | | | tumor |
| 49 | G208-8 | Breast | hom | 21.14 | 61 | 4 | No | Mostly well- |
| | | | control | | | | | differentiated, |
| | | | | | | | | focal solid |
| | | | | | | | | component |
| 51 | G227-6 | Breast | hom | 21.14 | 57 | 4 | No | Solid growth |
| | | | control | | | | | |
| 52 | G246-8 | Breast | hom | 21 | 50 | 3 | No | Mostly well- |
| | | | control | | | | | differentiated, |
| | | | | | | | | 2 focal |
| | | | | | | | | solid areas. |
| 53 | G256-6 | Breast | hom | 21.3 | 31 | 3 | No | Mostly |
| | | | control | | | | | DCIS/MIN, |
| | | | | | | | | large areas |
| | | | | | | | | of tubular |
| | | | | | | | | growth/early |
| | | | | | | | | carcinoma |
| 54 | G260-6 | Breast | hom | 21.3 | 22 | 1 | N/A | Normal breast |
| | | | control | | | | | parenchyma, |
| | | | | | | | | adenosis |
| | | | | | | | | with a |
| | | | | | | | | sclerosing |
| | | | | | | | | component |
| 55 | G278-3 | Breast | hom | 21.42 | 28 | 3 | No | Mostly tubular |
| | | | control | | | | | growth, |
| | | | | | | | | possible |
| | | | | | | | | DCIS on |
| | | | | | | | | periphery, |
| | | | | | | | | but may be |
| | | | | | | | | part of main |
| | | | | | | | | lesion |
| 83 | G184-8 | Breast | hom | 22.86 | | 4 | N/A | Mostly solid, |
| | | | control | | | | | cells uniform |
| | | | | | | | | and not too |
| | | | | | | | | pleomorphic, |
| | | | | | | | | foci of DCIS |
| | | | | | | | | with |
| | | | | | | | | comedonecrosis |
| 96 | G181-9 | Breast | hom | 26 | 44 | 4 | No | Mostly tubular |
| | | | control | | | | | growth, fused |
| | | | | | | | | in solid areas |

Heterozygous controls <20 weeks

| | | | | | | | | | |
|---|---|---|---|---|---|---|---|---|---|
| 4 | G183-4 | Breast | het | 18 | 73 | | 4 | Present | Multiple |
| | | | control | | | | | | foci of |
| | | | | | | | | | solid |
| | | | | | | | | | tumor with |
| | | | | | | | | | comedonecrosis |
| | | | | | | | | | seen |
| 6 | G128-8 | Breast | het | 18 | 14 | | 3 | 0 | Intermediate |
| | | | control | | | | | | grade tumor |
| 11 | G201-6 | Breast | het | 14 | 31 | | 3 | 0 | Microinvasion, |
| | | | control | | | | | | mostly |
| | | | | | | | | | intraepithelial |
| | | | | | | | | | neoplasia |
| 50 | G264-4 | Breast | het | 17.14 | | 13% (no | 1 | N/A | Adenosis |
| | | | control | | | tumor) | | | with a |
| | | | | | | | | | sclerosing |
| | | | | | | | | | component |
| 57 | G249-4 | Breast | het | 17.14 | | 23% (no | 1 | N/A | Normal breast |
| | | | control | | | tumor) | | | parenchyma, |
| | | | | | | | | | adenosis with |
| | | | | | | | | | a sclerosing |
| | | | | | | | | | component |
| 63 | G249-1 | Breast | het | 17.14 | 29 | | 3 | No | Well- |
| | | | control | | | | | | differentiated |
| 72 | G289-4 | Breast | het | 17.14 | | 14% (no | 1 | N/A | Normal breast |
| | | | control | | | tumor) | | | parenchyma, |
| | | | | | | | | | intraparenchymal |
| | | | | | | | | | LN |
| 73 | G299-3 | Breast | het | 17.14 | 58 | | 4 | No | Large solid |
| | | | control | | | | | | component with |
| | | | | | | | | | comedonecrosis |
| 75 | G311-4 | Breast | het | 17 | 30 | | 2 | N/A all | Low grade |
| | | | control | | | | | in situ | DCIS, low |
| | | | | | | | | | nuclear |
| | | | | | | | | | grade, no |
| | | | | | | | | | necrosis |
| 76 | G312-4 | Breast | het | 17 | 29 | | 1 | N/A | Atypical |
| | | | control | | | | | | ductal |
| | | | | | | | | | hyperplasia. |
| 84 | G288-8 | Breast | het | 17.14 | | | 3 | No | Minimally |
| | | | control | | | | | | invasive? |
| | | | | | | | | | Mostly DCIS? |
| 95 | G128-4 | Breast | het | 18.14 | 36 | | 2 | N/A all | DCIS, |
| | | | control | | | | | in situ | intermediate |
| | | | | | | | | | nuclear grade. |
| | | | | | | | | | Sclerosing |
| | | | | | | | | | adenosis |
| | | | | | | | | | also present. |

Heterozygous controls >20 weeks

| | | | | | | | | | |
|---|---|---|---|---|---|---|---|---|---|
| 8 | G144-8 | Breast | het | 20 | 46 | | 4 | Present | Abundant |
| | | | control | | | | | | apoptotic |
| | | | | | | | | | tumor cells |
| 23 | G202-9 | Breast | het | 21.14 | 48 | | 4 | Yes | Comedonecrosis |
| | | | control | | | | | | present |
| 29 | G236-1 | Breast | het | 21 | 56 | | 4 | Yes | Solid pattern |
| | | | control | | | | | | with minimal |
| | | | | | | | | | necrosis, no |
| | | | | | | | | | squamoid |
| | | | | | | | | | differentiation |
| 30 | G261-3 | Breast | het | 21.3 | 60 | | 3 | No | Primarily |
| | | | control | | | | | | MIN with |
| | | | | | | | | | microinvasion, |
| | | | | | | | | | tumor has |
| | | | | | | | | | tubular |
| | | | | | | | | | pattern and |
| | | | | | | | | | is low grade |
| 31 | G235-5 | Breast | het | 21 | 68 | | 4 | No, but | High grade |
| | | | control | | | | | continuous | tumor, mostly |
| | | | | | | | | tumor | solid and |
| | | | | | | | | | cribiform with |
| | | | | | | | | | extensive |
| | | | | | | | | | comedonecrosis |
| 65 | G288-4 | Breast | het | 21.14 | 52 | hotspots | 4 | No | Well- |
| | | | control | | | | | | differentiated |
| | | | | | | | | | and solid |
| | | | | | | | | | areas |
| 85 | G223-2 | Breast | het | 21.3 | 39 | | 4 | No | Mostly solid, |
| | | | control | | | | | | sheet-like |
| | | | | | | | | | growth |
| 86 | G232-1 | Breast | het | 21 | 35 | | 3 | No | Tubular |
| | | | control | | | | | | growth, well- |
| | | | | | | | | | differentiated |
| 87 | G236-1 | Breast | het | 21 | | | 4 | No | Solid growth |
| | | | control | | | | | | with numerous |
| | | | | | | | | | mitotic |
| | | | | | | | | | figures |
| 94 | G144-8 | Breast | het | 20 | | | 4 | No | Solid tumor |
| | | | control | | | | | | with a small |
| | | | | | | | | | amount of |
| | | | | | | | | | comedonecrosis |

| | | | | | | | | | |
|---|---|---|---|---|---|---|---|---|---|
| | | | | | | | | Presence of | |
| Heterozygous | Mouse | Tissue | | Age of | Ki-67, | | Grade of worst | satellite | |
| KO | number | type | Genotype | mouse | % | Ki-67 | breast lesion* | nodules | Comments |

Heterozygous KO

| | | | | | | | | |
|---|---|---|---|---|---|---|---|---|
| 77 | G271-5 | Breast | het KO | 17.86 | 52 | 4 | N/A | Solid component |
| | | | | | | | | is mitotically |
| | | | | | | | | active |
| 78 | G290-3 | Breast | het KO | 17.14 | 30 | 2 | N/A all in situ | Low grade |
| | | | | | | | | DCIS, low |
| | | | | | | | | nuclear grade, |
| | | | | | | | | no necrosis |
| 79 | G292-6 | Breast | het KO | 17.14 | 34 | 4 | No | Mostly tubular |
| | | | | | | | | growth, some |
| | | | | | | | | fusion/solid |
| | | | | | | | | component |
| 80 | G297-4 | Breast | het KO | 17.14 | 34 | 2 | N/A all in situ | Low grade |
| | | | | | | | | DCIS, low |
| | | | | | | | | nuclear |
| | | | | | | | | grade, no |
| | | | | | | | | necrosis, |
| | | | | | | | | sclerosing |
| | | | | | | | | adenosis? |
| 81 | G298-1 | Breast | het KO | 17.14 | 26 | 1 | N/A | Adenosis |
| 82 | G306-4 | Breast | het KO | 17 | 35 | 3 | N/A | Focus of |
| | | | | | | | | tumor with |
| | | | | | | | | surrounding |
| | | | | | | | | normal |
| | | | | | | | | appearing |
| | | | | | | | | breast |
| | | | | | | | | parenchyma |
| | | | | | | | | and |
| | | | | | | | | intraparenchymal |
| | | | | | | | | LN |

Heterozygous KO >20 weeks

| | | | | | | | | | |
|---|---|---|---|---|---|---|---|---|---|
| 1 | G129-8 | Breast | het KO | 26 | 17 | | 3 | 0 | A late early |
| | | | | | | | | | or an early |
| | | | | | | | | | late |
| | | | | | | | | | carcinoma - |
| | | | | | | | | | although |
| | | | | | | | | | the invasive |
| | | | | | | | | | component |
| | | | | | | | | | is large, |
| | | | | | | | | | the tumor is |
| | | | | | | | | | mostly well- |
| | | | | | | | | | differentiated |
| | | | | | | | | | with |
| | | | | | | | | | predominantly |
| | | | | | | | | | tubular/acinar |
| 12 | G164-3 | Breast | het KO | 23.9 | 36 | | 4 | Present | Well and |
| | | | | | | | | | poorly |
| | | | | | | | | | differentiated |
| | | | | | | | | | areas, focal |
| | | | | | | | | | papillary |
| | | | | | | | | | growth pattern |
| 13 | G164-1 | Breast | het KO | 23.9 | 52 | | 4 | Present | Mostly well- |
| | | | | | | | | | differentiated, |
| | | | | | | | | | two high grade |
| | | | | | | | | | foci with |
| | | | | | | | | | solid growth |
| | | | | | | | | | and |
| | | | | | | | | | comedonecrosis |
| 14 | G165-1 | Breast | het KO | 25.6 | 49 | avg (lower | 4 | Present | Mostly tubular |
| | | | | | | areas and | | | growth, focal |
| | | | | | | hotspots | | | endophytic |
| | | | | | | of >90% | | | papillary |
| | | | | | | staining) | | | growth, focal |
| | | | | | | | | | solid areas |
| | | | | | | | | | (don't |
| | | | | | | | | | make up |
| | | | | | | | | | discrete |
| | | | | | | | | | nodules) |
| 22 | G199-5 | Breast | het KO | 21.14 | 72 | | 4 | Yes | Squamous |
| | | | | | | | | | differentiation |
| | | | | | | | | | of de- |
| | | | | | | | | | differentiated |
| | | | | | | | | | tumor |
| 27 | G251-9 | Breast | het KO | 21 | 34 | | 3 | No | Mostly tubular |
| | | | | | | | | | growth |
| 89 | G251-9 | Breast | het KO | 21 | | | 4 | No | Tubular growth, |
| | | | | | | | | | coalescing to |
| | | | | | | | | | areas of |
| | | | | | | | | | solid growth |
| 90 | G257-9 | Breast | het KO | 21.3 | 42 | | 2 | N/A all | High grade |
| | | | | | | | | in situ | DCIS |
| 91 | G258-6 | Breast | het KO | 21.3 | 47 | | 4 | No, all | Mostly solid |
| | | | | | | | | continuous | tumor growth |
| | | | | | | | | tumor | |
| 92 | G282-6 | Breast | het KO | 21 | 56 | | 4 | No, all | Solid growth, |
| | | | | | | | | continuous | highly |
| | | | | | | | | tumor | mitotically |
| | | | | | | | | | active |
| 93 | G287-9 | Breast | het KO | 21 | | 9% (no | 0 | N/A | Normal breast |
| | | | | | | tumor) | | | parenchyma |
| 97 | G185-3 | Breast | het KO | 26 | 59 | | 4 | No, all | High grade |
| | | | | | | | | continuous | tumor with |
| | | | | | | | | tumor | extensive |
| | | | | | | | | | necrosis |

| | | | | | | | | | |
|---|---|---|---|---|---|---|---|---|---|
| | | | | | | | | Presence of | |
| Homozygous | Mouse | Tissue | | Age of | Ki-67, | | Grade of worst | satellite | |
| KO | number | type | Genotype | mouse | % | Ki-67 | breast lesion* | nodules | Comments |

Homozygous KO <20 weeks

| | | | | | | | | | |
|---|---|---|---|---|---|---|---|---|---|
| 10 | G180-8 | Breast | hom KO | 15.9 | 66.00 | | 4 | Present | Focal areas |
| | | | | | | | | | with a well- |
| | | | | | | | | | differentiated |
| | | | | | | | | | tubular growth |
| | | | | | | | | | pattern, but |
| | | | | | | | | | also contains |
| | | | | | | | | | areas with |
| | | | | | | | | | solid growth |
| | | | | | | | | | and |
| | | | | | | | | | comedonecrosis, |
| | | | | | | | | | overall |
| | | | | | | | | | intermediate |
| | | | | | | | | | grade but |
| | | | | | | | | | has low and |
| | | | | | | | | | higher grade |
| | | | | | | | | | components |
| 24 | G213-10 | Breast | hom KO | 17 | | | 3 | No | mostly |
| | | | | | | | | | MIN/DCIS, |
| | | | | | | | | | microinvasion |
| 25 | G215-6 | Breast | hom KO | 17 | | | 4 | No | High mitotic |
| | | | | | | | | | activity in |
| | | | | | | | | | solid areas |
| 26 | G216-4 | Breast | hom KO | 17 | | | 2 | No | Adenosis with |
| | | | | | | | | | foci of MIN |
| | | | | | | | | | present, no |
| | | | | | | | | | carcinoma |
| 35 | G213-10 | Breast | hom KO | 17 | 69.00 | representative | 4 | No, but | Well-formed |
| | | | | | | tumor, up to | | continuous | neoplastic |
| | | | | | | 90% focally | | tumor | glands with |
| | | | | | | | | | solid tumor |
| | | | | | | | | | within the |
| | | | | | | | | | center of the |
| | | | | | | | | | section of |
| | | | | | | | | | higher |
| | | | | | | | | | nuclear grade |
| 36 | G215-6 | Breast | hom KO | 17 | 68.00 | | 4 | No | High grade |
| | | | | | | | | | nuclei, single |
| | | | | | | | | | cell necrosis, |
| | | | | | | | | | abundant |
| | | | | | | | | | mitotic |
| | | | | | | | | | activity |
| 37 | G216-4 | Breast | hom KO | 17 | 62.00 | in DCIS area | 2 | N/A all | Solid and |
| | | | | | | | | in situ | cribriform |
| | | | | | | | | | type MIN/DCIS |
| 38 | G275-1 | Breast | hom KO | 17.14 | 42.00 | | 3 | No | Well- |
| | | | | | | | | | differentiated, |
| | | | | | | | | | tubular growth |
| 39 | G277-4 | Breast | hom KO | 17.14 | 21.00 | | 1 | No | Adenosis |
| 40 | G295-9 | Breast | hom KO | 17.14 | 58.00 | | 4 | No | Solid tumor |
| | | | | | | | | | with extensive |
| | | | | | | | | | necrosis |
| 41 | G315-9 | Breast | hom KO | 17 | 45.00 | | 2 | N/A all | DCIS, small |
| | | | | | | | | in situ | amount of |
| | | | | | | | | | comedonecrosis |

Homozygous KO >20 weeks

| | | | | | | | | |
|---|---|---|---|---|---|---|---|---|
| 2 | G140-8 | Breast | hom KO | 26 | 45 | 4 | Present | Anaplastic |
| | | | | | | | | tumor with |
| | | | | | | | | extensive |
| | | | | | | | | solid |
| 3 | G125-6 | Breast | hom KO | 26 | 58 | 4 | Present | Predominantly |
| | | | | | | | | solid growth, |
| | | | | | | | | necrotic |
| | | | | | | | | focus ~1% of |
| | | | | | | | | total tumor |
| 5 | G174-1 | Breast | hom KO | 20 | 28 | 3 | 0 | Single |
| | | | | | | | | tumor with |
| | | | | | | | | predominantly |
| | | | | | | | | tubular/aciner |
| | | | | | | | | formations |
| 9 | G145-8 | Breast | hom KO | 21.9 | 48 | 4 | Present | Abundant |
| | | | | | | | | comedonecrosis |
| | | | | | | | | and apoptotic |
| | | | | | | | | tumor cells |
| 19 | G207-3 | Breast | hom KO | 21.14 | 37 | 3 | No | Predominantly |
| | | | | | | | | tubular pattern, |
| | | | | | | | | single cell |
| | | | | | | | | apoptoses present |
| 21 | G209-1 | Breast | hom KO | 21.14 | 54 | 3 | No | Predominantly |
| | | | | | | | | tubular growth |
| | | | | | | | | pattern |
| 58 | G231-1 | Breast | hom KO | 21 | 56 | 3 | No | Well- |
| | | | | | | | | differentiated, |
| | | | | | | | | difficult to |
| | | | | | | | | determine |
| | | | | | | | | if invasive, |
| | | | | | | | | but appears |
| | | | | | | | | to be a |
| | | | | | | | | carcinoma |
| | | | | | | | | with tubular |
| | | | | | | | | growth pattern |
| 59 | G234-6 | Breast | hom KO | 21 | 36 | 4 | No | Sheet-like |
| | | | | | | | | growth and |
| | | | | | | | | areas of well- |
| | | | | | | | | differentiated, |
| | | | | | | | | tubular growth |
| 60 | G267-3 | Breast | hom KO | 21.14 | 76 | 4 | Yes | High grade |
| | | | | | | | | tumor with |
| | | | | | | | | extensive |
| | | | | | | | | necrosis |
| 61 | G269-8 | Breast | hom KO | 21.3 | 39 | 3 | No | Well- |
| | | | | | | | | differentiated, |
| | | | | | | | | mostly tubular |
| | | | | | | | | growth |
| 64 | G268 | Breast | hom KO | 21.3 | 47 | 4 | No | Sheet-like |
| | | | | | | | | growth, lobular |
| | | | | | | | | and squamoid |
| 88 | G190-4 | Breast | hom KO | 25.42 | 41 | 2 | N/A all | DCIS, small |
| | | | | | | | in situ | amount of |
| | | | | | | | | comedonecrosis |
| 98 | G125-6 | Breast | hom KO | 26 | | 4 | No, all | Mostly solid |
| | | | | | | | continuous | growth, sheet- |
| | | | | | | | tumor | like |

Grading of breast lesion:
0 Normal breast parenchyma
1 Hyperplasia—densely packed lobules, basement membrane intact, bland cytology but increased N/C ratio
2 Mammary intraepithelial neoplasia—florid epithelial proliferation, basement membrane intact, leucocyte infiltration
3 Early carcinoma—early stromal infiltration, nuclearpleomorphism, looks like HG DCIS with early stromal invasion
4 Late carcinoma—solid sheet growth, lack of acini, prominent nucleoli, multiple onvasice tumor nodules

TABLE 10
A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 1802.24 | 2402.99 | 2603.24 | 3003.74 | 3604.49 | 3804.74 | 4004.98 |
| | | G202_fl.wt.cre2 | 1020.717 | 2211.553 | 2211.553 | 3062.15 | 3062.15 | 3232.27 | 3742.628 |
| | | G202_fl.wt.cre3 | 2689.67 | 2868.98 | 3944.85 | 4303.48 | 4482.79 | 5020.72 | 5200.03 |
| fl.wt.cre+ | G251 | G251_fl.wt.cre1 | 1979.265 | 3016.023 | 3016.023 | 3204.524 | 3581.527 | 2581.527 | 3958.53 |
| | | G251_fl.wt.cre2 | 2418.71 | 3325.72 | 3325.72 | 3476.89 | 33476.89 | 4081.56 | 4232.73 |
| | | G251_fl.wt.cre3 | 3847.895 | 4074.242 | 4187.415 | 4526.935 | 6677.23 | 7016.75 | 7016.75 |
| fl/fl Cre− | G184 | G184_fl.fl.cre1 | 1170.899 | 1405.079 | 2107.619 | 2107.619 | 2107.619 | 2341.799 | 2575.978 |
| | | G184_fl.fl.cre1 | 1730.01 | 2595.015 | 2595.015 | 2811.267 | 2811.267 | 3027.518 | 3892.523 |
| | | G184_fl.fl.cre1 | 3662.109 | 3662.109 | 4150.391 | 4638.672 | 4638.672 | 5126.953 | 5126.953 |
| fl/fl Cre+ | G209 | G209_fl.fl.cre1 | 828.178 | 1757.354 | 1979.548 | 2060.346 | 2080.545 | 2262.341 | 2545.133 |
| | | G209_fl.fl.cre1 | 17556.23 | 642.301 | 1420.848 | 1790.657 | 1868.512 | 2763.841 | 4106.834 |
| | | G209_fl.fl.cre1 | 8621.074 | 8901.76 | 10565.83 | 13312.54 | 14736.02 | 16680.78 | 23317 |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 4004.98 | 4205.23 | 5006.23 | 5206.48 | 5406.73 | 5606.98 | 5807.23 |
| | | G202_fl.wt.cre2 | 3912.748 | 4082.867 | 4252.987 | 4423.106 | 4423.106 | 4423.106 | 4933.464 |
| | | G202_fl.wt.cre3 | 5737.97 | 5737.97 | 5917.28 | 5917.28 | 6096.59 | 6096.59 | 6096.59 |
| fl.wt.cre+ | G251 | G251_fl.wt.cre1 | 3958.53 | 4147.031 | 5183.789 | 5466.541 | 5843.544 | 6032.045 | 7634.307 |
| | | G251_fl.wt.cre2 | 4837.41 | 5744.42 | 6046.76 | 6197.93 | 7104.95 | 7256.11 | 7407.28 |
| | | G251_fl.wt.cre3 | 7469.443 | 7922.137 | 8374.83 | 9619.737 | 10638.3 | 11090.99 | 11883.21 |
| fl/fl Cre− | G184 | G184_fl.fl.cre1 | 2575.978 | 2810.158 | 2810.158 | 2810.158 | 3044.338 | 3044.338 | 3278.518 |
| | | G184_fl.fl.cre1 | 4108.774 | 4108.774 | 4325.026 | 4325.026 | 4325.026 | 4325.026 | 4325.026 |
| | | G184_fl.fl.cre1 | 5126.953 | 5615.234 | 6103.516 | 6347.656 | 6347.656 | 6347.656 | 6591.797 |
| fl/fl Cre+ | G209 | G209_fl.fl.cre1 | 3191.516 | 3373.311 | 3494.508 | 3777.301 | 4060.093 | 4140.891 | 4625.679 |
| | | G209_fl.fl.cre1 | 4612.889 | 4768.599 | 4943.772 | 5644.464 | 6637.111 | 7435.121 | 8602.941 |
| | | G209_fl.fl.cre1 | 70291.82 | 71374.47 | 79754.96 | 119391.8 | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 5807.23 | 5807.23 | 5807.23 | 6007.48 | 6207.73 | 6407.97 | 6608.22 |
| | | G202_fl.wt.cre2 | 4933.464 | 5103.584 | 5443.823 | 5443.823 | 5613.942 | 5613.942 | 5613.942 |
| | | G202_fl.wt.cre3 | 6096.59 | 6455.21 | 6634.52 | 6813.84 | 7351.77 | 7710.39 | 7710.39 |
| fl.wt.cre+ | G251 | G251_fl.wt.cre1 | 7917.059 | 8011.31 | 8011.31 | 8105.561 | 8671.065 | 8671.065 | 9330.82 |
| | | G251_fl.wt.cre2 | 8011.96 | 8011.96 | 8314.3 | 8918.97 | 9372.48 | 9372.48 | 9825.99 |
| | | G251_fl.wt.cre3 | 13354.46 | 13580.81 | 13693.98 | 14712.54 | 15052.06 | 16296.97 | 16636.49 |
| fl/fl Cre− | G184 | G184_fl.fl.cre1 | 3278.518 | 3746.878 | 3746.878 | 3981.057 | 3981.057 | 4215.237 | 4215.237 |
| | | G184_fl.fl.cre1 | 4541.277 | 4757.528 | 4757.528 | 4973.78 | 5190.031 | 5406.282 | 5406.282 |
| | | G184_fl.fl.cre1 | 6581.797 | 7324.219 | 7568.359 | 7568.359 | 7812.5 | 8056.641 | 8056.641 |
| fl/fl Cre+ | G209 | G209_fl.fl.cre1 | 5332.66 | 6665.825 | 7736.397 | 8746.37 | 10180.53 | 11473.3 | 15775.79 |
| | | G209_fl.fl.cre1 | 9498.27 | 9848.616 | 10568.77 | 14364.19 | 16018.6 | 26022.92 | 26198.1 |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 6608.22 | 6608.22 | 6808.47 | 7208.97 | 7609.47 | 7609.47 | 8009.97 |
| | | G202_fl.wt.cre2 | 5954.181 | 6124.301 | 6464.54 | 6634.659 | 6634.659 | 6804.778 | 7145.017 |
| | | G202_fl.wt.cre3 | 7889.7 | 8248.33 | 8427.64 | 8427.64 | 8427.64 | 8427.64 | 8427.64 |
| fl.wt.cre+ | G251 | G251_fl.wt.cre1 | 9802.074 | 10179.08 | 10838.83 | 10933.08 | 11121.58 | 11310.09 | 11404.34 |
| | | G251_fl.wt.cre2 | 9977.16 | 10884.2 | 11035.3 | 12395.9 | 12395.9 | 12395.9 | 12395.9 |
| | | G251_fl.wt.cre3 | 17315.53 | 17768.22 | 18220.91 | 18220.91 | 18899.96 | 19239.48 | 19465.82 |
| fl/fl Cre− | G184 | G184_fl.fl.cre1 | 4683.597 | 4683.597 | 4917.777 | 5151.957 | 5151.957 | 5151.957 | 5386.137 |
| | | G184_fl.fl.cre1 | 5838.785 | 6055.036 | 6487.539 | 6487.539 | 6271.287 | 6271.287 | 6271.287 |
| | | G184_fl.fl.cre1 | 8056.641 | 8300.781 | 8300.781 | 8544.922 | 8789.062 | 8789.062 | 8789.062 |
| fl/fl Cre+ | G209 | G209_fl.fl.cre1 | 20138.87 | 20219.67 | 20381.27 | 24441.36 | 29390.23 | 34985.48 | 35833.86 |
| | | G209_fl.fl.cre1 | 27307.53 | 30402.25 | 31881.49 | 32445.93 | 35190.31 | 44046.28 | 50508.22 |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 8210.22 | 8210.22 | 8610.72 | 9011.21 | 9211.46 | 9211.46 | 9211.46 |
| | | G202_fl.wt.cre2 | 7145.017 | 7145.017 | 7315.137 | 7315.137 | 7655.376 | 7825.495 | 8335.854 |
| | | G202_fl.wt.cre3 | 8606.95 | 8786.26 | 8965.57 | 9503.51 | 10041.4 | 10220.8 | 10220.8 |
| fl.wt.cre+ | G251 | G251_fl.wt.cre1 | 12441.09 | 15174.36 | 15268.62 | 17059.38 | 17813.38 | 18378.89 | 18567.39 |
| | | G251_fl.wt.cre2 | 13000.5 | 13000.5 | 13454 | 814512.2 | 14814.6 | 15570.4 | 15721.6 |
| | | G251_fl.wt.cre3 | 19692.17 | 21050.25 | 21842.46 | 26822.09 | 27048.44 | 28406.52 | 28519.69 |
| fl/fl Cre− | G184 | G184_fl.fl.cre1 | 5386.137 | 5620.316 | 5620.316 | 5620.316 | 5620.316 | 5620.316 | 5620.316 |
| | | G184_fl.fl.cre1 | 6271.287 | 6055.036 | 6055.036 | 6055.036 | 6055.036 | 6703.79 | 6703.79 |
| | | G184_fl.fl.cre1 | 9277.344 | 9277.344 | 9521.484 | 9765.625 | 10009.77 | 10009.77 | 10253.91 |
| fl/fl Cre+ | G209 | G209_fl.fl.cre1 | 83444.01 | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 9411.71 | 9411.71 | 9611.96 | 9812.21 | 10012.5 | 10012.5 | 10212.7 |
| | | G202_fl.wt.cre2 | 8335.854 | 8505.973 | 8505.973 | 8505.973 | 8846.212 | 8846.212 | 9186.451 |
| | | G202_fl.wt.cre3 | 10400.1 | 10579.4 | 10579.4 | 10758.7 | 11117.3 | 112966 | 11655.2 |
| fl.wt.cre+ | G251 | G251_fl.wt.cre1 | 20358.2 | 20452.4 | 21394.9 | 24599.4 | 26672.95 | 26767.2 | 28275.21 |
| | | G251_fl.wt.cre2 | 16326.3 | 16779.8 | 17233.3 | 18896.1 | 19198.5 | 19652 | 19803.1 |
| | | G251_fl.wt.cre3 | 28632.87 | 29311.91 | 331462.2 | 31914.89 | 32367.59 | 32933.45 | 33612.49 |
| fl/fl Cre− | G184 | G184_fl.fl.cre1 | 5620.316 | 5854.496 | 5854.496 | 5854.496 | 6088.676 | 6088.676 | 6322.856 |
| | | G184_fl.fl.cre1 | 6703.79 | 7136.292 | 7352.544 | 7785.046 | 8001.298 | 8217.549 | 8217.549 |
| | | G184_fl.fl.cre1 | 10253.91 | 10498.05 | 10742.19 | 10986.33 | 10986.33 | 11230.47 | 11474.61 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 10413 | 10613.2 | 10813.5 | 11013.7 | 11013.7 | 11214 | 11414.2 |
| | | G202_fl.wt.cre2 | 9356.57 | 9356.57 | 10037.05 | 10547.41 | 10547.41 | 10717.53 | 10887.65 |
| | | G202_fl.wt.cre3 | 11834.6 | 12372.5 | 12551.8 | 12910.4 | 13448.4 | 13448.4 | 13807 |
| fl.wt.cre+ | G251 | G251_fl.wt.cre1 | 34401.51 | 35721.02 | 36286.52 | 37794.53 | 38077.29 | 45805.8 | 48916.1 |
| | | G251_fl.wt.cre2 | 23128.9 | 23431.2 | 23431.2 | 25547.6 | 25849.9 | 30536.1 | 834768.9 |
| | | G251_fl.wt.cre3 | 33838.84 | 34291.54 | 34631.06 | 38931.64 | 40402.9 | 41647.8 | 41874.15 |
| fl/fl Cre− | G184 | G184_fl.fl.cre1 | 6322.856 | 6557.036 | 6557.036 | 6557.036 | 6557.036 | 6557.036 | 6557.036 |
| | | G184_fl.fl.cre1 | 8650.051 | 8866.303 | 8866.303 | 8866.303 | 9082.554 | 9731.308 | 9731.308 |
| | | G184_fl.fl.cre1 | 11718.75 | 11718.75 | 11962.89 | 11962.89 | 11962.89 | 12207.03 | 12207.03 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 11414.2 | 11614.5 | 11814.7 | 12415.5 | 12615.7 | 13016.2 | 13216.4 |
| | | G202_fl.wt.cre2 | 11398 | 11738.24 | 12078.48 | 12248.6 | 12588.84 | 12758.96 | 12929.08 |
| | | G202_fl.wt.cre3 | 13986.3 | 14344.9 | 14344.9 | 14524.2 | 14524.2 | 14703.5 | 15062.2 |
| fl.wt.cre+ | G251 | G251_fl.wt.cre1 | 51272.39 | 55984.92 | 67672.01 | 70216.78 | 85014.14 | 98680.49 | 115645.6 |
| | | G251_fl.wt.cre2 | 35071.2 | 36885.2 | 39455.1 | 42176.2 | 43990.2 | 44141.4 | 345199.5 |
| | | G251_fl.wt.cre3 | 42892.71 | 49230.42 | 56926.21 | 64169.31 | 66659.12 | 68583.07 | 77750.11 |
| fl/fl Cre− | G184 | G184_fl.fl.cre1 | 6791.216 | 6791.216 | 6791.216 | 6791.216 | 7259.575 | 7493.755 | 7493.755 |
| | | G184_fl.fl.cre1 | 9731.308 | 9731.308 | 9731.308 | 9947.559 | 10163.81 | 10380.06 | 10596.31 |
| | | G184_fl.fl.cre1 | 12939.45 | 13427.73 | 13427.73 | 13427.73 | 13427.73 | 13671.88 | 13671.88 |
| fl/fl Cre+ | G209 | G209_fl.fl.cre1 | 374922.4 | | | | | | |
| | | G209_fl.fl.cre1 | 437095.6 | | | | | | |
| | | G209_fl.fl.cre1 | 436948.1 | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 13817.2 | 13817.2 | 14017.4 | 14017.4 | 14017.4 | 14017.4 | 14217.7 |
| | | G202_fl.wt.cre2 | 13779.68 | 14119.92 | 14460.15 | 14800.39 | 15480.87 | 16501.59 | 16841.83 |
| | | G202_fl.wt.cre3 | 15241.5 | 15420.8 | 16138 | 16676 | 17213.9 | 17213.9 | 17572.5 |
| fl.wt.cre+ | G251 | G251_fl.wt.cre1 | 130443 | 132139.5 | 139396.8 | 152214.9 | 154099.9 | 206409 | 254005.7 |
| | | G251_fl.wt.cre2 | 48071.8 | 49129.9 | 53967.3 | 54118.5 | 55630.2 | 55630.2 | 56688.4 |
| | | G251_fl.wt.cre3 | 86464.46 | 92123.13 | 93368.04 | 113852.4 | 122679.9 | 187415.1 | 233250.3 |
| fl/fl Cre− | G184 | G184_fl.fl.cre1 | 7727.935 | 7727.935 | 7727.935 | 7727.935 | 7962.115 | 7962.115 | 7962.115 |
| | | G184_fl.fl.cre1 | 10596.31 | 10596.31 | 10596.31 | 10596.31 | 10812.56 | 10812.56 | 10812.56 |
| | | G184_fl.fl.cre1 | 13916.02 | 14160.16 | 14892.58 | 14892.58 | 14892.58 | 14892.58 | 15136.72 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 14217.7 | 14217.7 | 14417.9 | 15018.7 | 15419.2 | 15819.7 | 16220.2 |
| | | G202_fl.wt.cre2 | 17182.07 | 17862.54 | 17862.54 | 18032.66 | 18372.9 | 18372.9 | 18713.14 |
| | | G202_fl.wt.cre3 | 17751.8 | 18469.1 | 18827.7 | 197243 | 19903.6 | 20082.9 | 20620.8 |
| fl.wt.cre+ | G251 | G251_fl.wt.cre1 | 501319.5 | | | | | | |
| | | G251_fl.wt.cre2 | 56839.6 | 61223.5 | 66060.9 | 69840.1 | 699913 | 76491.5 | 77700.9 |
| | | G251_fl.wt.cre3 | 357175.2 | 676097.8 | | | | | |
| fl/fl Cre− | G184 | G184_fl.fl.cre1 | 8196.295 | 8430.475 | 8430.475 | 8664.654 | 8664.654 | 8664.654 | 8898.834 |
| | | G184_fl.fl.cre1 | 11245.07 | 11245.07 | 11245.07 | 11461.32 | 11677.57 | 11893.82 | 12110.07 |
| | | G184_fl.fl.cre1 | 15380.86 | 15380.86 | 15380.86 | 15380.86 | 15869.14 | 16113.28 | 16113.28 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 16220.2 | 16420.4 | 16420.4 | 16420.4 | 16620.7 | 16820.9 | 17021.2 |
| | | G202_fl.wt.cre2 | 18883.26 | 18883.26 | 19053.38 | 19053.38 | 19053.38 | 19733.86 | 20074.1 |
| | | G202_fl.wt.cre3 | 21158.8 | 21158.8 | 21696.7 | 22413.9 | 22593.2 | 22951.9 | 231312 |
| fl.wt.cre+ | G251 | G251_fl.wt.cre1 | | | | | | | |
| | | G251_fl.wt.cre2 | 88887.4 | 135599 | 153437 | 165984 | 196217 | 261825 | 269232 |
| | | G251_fl.wt.cre3 | | | | | | | |
| fl/fl Cre− | G184 | G184_fl.fl.cre1 | 8898.834 | 8898.834 | 8898.834 | 8898.834 | 9133.014 | 9601.374 | 9601.374 |
| | | G184_fl.fl.cre1 | 12542.57 | 12758.83 | 12758.83 | 12758.83 | 13191.33 | 13191.33 | 13407.58 |
| | | G184_fl.fl.cre1 | 16357.42 | 16601.56 | 16845.7 | 17089.84 | 17822.27 | 18066.41 | 18066.41 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 17221.4 | 17421.7 | 18022.4 | 18022.4 | 18422.9 | 18823.4 | 19824.7 |
| | | G202_fl.wt.cre2 | 20074.1 | 20244.22 | 20754.57 | 21094.81 | 21264.93 | 21435.05 | 22115.53 |
| | | G202_fl.wt.cre3 | 23310.5 | 24565.7 | 24924.3 | 25103.6 | 25641.5 | 25820.8 | 26000.2 |
| fl.wt.cre+ | G251 | G251_fl.wt.cre1 | 584420 | | | | | | |
| | | G251_fl.wt.cre2 | | | | | | | |
| | | G251_fl.wt.cre3 | | | | | | | |
| fl/fl Cre− | G184 | G184_fl.fl.cre1 | 9601.374 | 9601.374 | 9835.554 | 9835.554 | 9835.554 | 10069.73 | 10069.73 |
| | | G184_fl.fl.cre1 | 13623.83 | 13623.83 | 13623.83 | 13623.83 | 13840.08 | 13840.08 | 13840.08 |
| | | G184_fl.fl.cre1 | 18066.41 | 18310.55 | 18554.69 | 18554.69 | 19531.25 | 19775.39 | 19775.39 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 19824.7 | 20425.4 | 21026.2 | 21226.4 | 21426.7 | 21827.2 | 21827.2 |
| | | G202_fl.wt.cre2 | 22115.53 | 22625.89 | 22625.89 | 22796.01 | 22966.13 | 22966.13 | 23136.25 |
| | | G202_fl.wt.cre3 | 27255.3 | 27434.7 | 27614 | 27793.3 | 27972.6 | 28151.9 | 28331.2 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 10069.73 | 10069.73 | 10303.91 | 10303.91 | 10303.91 | 10303.91 | 10538.09 |
| | | G184_fl.fl.cre1 | 14056.33 | 14272.59 | 14272.59 | 14272.59 | 14705.09 | 14705.09 | 14921.34 |
| | | G184_fl.fl.cre1 | 20019.53 | 20263.67 | 20263.67 | 20751.95 | 20751.95 | 21240.23 | 21728.52 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 22227.7 | 23028.7 | 23028.7 | 23429.2 | 23829.7 | 25231.4 | 25832.1 |
| | | G202_fl.wt.cre2 | 23306.37 | 23476.49 | 23646.61 | 23986.84 | 24156.96 | 24156.96 | 24156.96 |
| | | G202_fl.wt.cre3 | 28510.5 | 29048.5 | 29586.4 | 29765.7 | 29765.7 | 30482.9 | 30841.6 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 10538.09 | 10772.27 | 11006.45 | 11240.63 | 11474.81 | 11474.81 | 11708.99 |
| | | G184_fl.fl.cre1 | 15137.59 | 15137.59 | 15137.59 | 15353.84 | 15353.84 | 15570.09 | 15786.34 |
| | | G184_fl.fl.cre1 | 22216.8 | 22216.8 | 22705.08 | 22705.08 | 22949.22 | 23193.36 | 23193.36 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 26232.6 | 27033.6 | 27233.9 | 27834.6 | 28034.9 | 28435.4 | 28435.4 |
| | | G202_fl.wt.cre2 | 24327.08 | 24497.2 | 25347.8 | 25347.8 | 25688.04 | 25688.04 | 26538.64 |
| | | G202_fl.wt.cre3 | 31020.9 | 32455.4 | 32634.7 | 33172.6 | 33351.9 | 33710.6 | 33710.6 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 11943.17 | 12177.35 | 12177.35 | 12177.35 | 12411.53 | 12411.53 | 12879.89 |
| | | G184_fl.fl.cre1 | 15786.34 | 16002.6 | 16002.6 | 16218.85 | 16651.35 | 16651.35 | 16651.35 |
| | | G184_fl.fl.cre1 | 23437.5 | 23925.78 | 24414.06 | 24902.34 | 24902.34 | 25390.63 | 25634.77 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 28635.6 | 29036.1 | 29236.4 | 30037.4 | 31439.1 | 31439.1 | 31639.4 |
| | | G202_fl.wt.cre2 | 27389.23 | 28069.71 | 28239.83 | 31472.1 | 31642.22 | 32492.82 | 32662.94 |
| | | G202_fl.wt.cre3 | 34607.1 | 34786.4 | 3496537 | 37655.4 | 38014 | 383726 | 38731.3 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 13114.07 | 13114.07 | 13114.07 | 13348.25 | 13816.61 | 14050.79 | 14050.79 |
| | | G184_fl.fl.cre1 | 17300.1 | 17516.35 | 18165.11 | 18597.61 | 18597.61 | 18597.61 | 19030.11 |
| | | G184_fl.fl.cre1 | 25634.77 | 25878.91 | 26123.05 | 26123.05 | 26367.19 | 26367.19 | 26611.33 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 32640.6 | 32640.6 | 32840.9 | 33241.4 | 35043.6 | 35844.6 | 36645.6 |
| | | G202_fl.wt.cre2 | 32662.94 | 32833.06 | 33003.18 | 33853.77 | 33853.77 | 35725.09 | 37766.52 |
| | | G202_fl.wt.cre3 | 39269.2 | 39986.5 | 42855.4 | 43214.1 | 43393.4 | 44110.6 | 44289.9 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 14050.79 | 14050.79 | 14284.97 | 14519.15 | 14753.33 | 14753.33 | 14753.33 |
| | | G184_fl.fl.cre1 | 19030.11 | 19030.11 | 19246.36 | 19462.62 | 19678.87 | 19678.87 | 19678.87 |
| | | G184_fl.fl.cre1 | 27099.61 | 27343.75 | 27343.75 | 27587.89 | 28076.17 | 28076.17 | 28320.31 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 36845.9 | 37446.6 | 38848.3 | 38848.3 | 39048.6 | 39449.1 | 39849.6 |
| | | G202_fl.wt.cre2 | 38447 | 38617.12 | 39127.48. | 39978.07 | 40148.19 | 41168.91 | 42529.87 |
| | | G202_fl.wt.cre3 | 44289.9 | 44289.9 | 44827.9 | 45903.7 | 47158.9 | 47876.2 | 49131.3 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 14753.33 | 14753.33 | 14987.51 | 15455.87 | 15455.87 | 16158.41 | 16626.77 |
| | | G184_fl.fl.cre1 | 19678.87 | 19895.12 | 19895.12 | 20111.37 | 20111.37 | 20111.37 | 20111.37 |
| | | G184_fl.fl.cre1 | 28808.59 | 29052.73 | 29296.88 | 29296.88 | 29785.16 | 30273.44 | 30761.72 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 40450.3 | 40850.8 | 41651.8 | 41852.1 | 42052.3 | 42853.3 | 43454.1 |
| | | G202_fl.wt.cre2 | 43210.34 | 43720.7 | 43890.82 | 44060.94 | 44741.42 | 45251.78 | 46102.37 |
| | | G202_fl.wt.cre3 | 50027.9 | 50207.2 | 50565.8 | 51103.8 | 53255.5 | 53977.7 | 54331.4 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 16626.77 | 16860.95 | 16860.95 | 18031.85 | 18266.03 | 18266.03 | 18500.21 |
| | | G184_fl.fl.cre1 | 20327.62 | 20543.87 | 20543.87 | 21192.63 | 21408.88 | 21408.88 | 21408.88 |
| | | G184_fl.fl.cre1 | 30761.72 | 31005.868 | 31250 | 31738.28 | 31738.28 | 31982.42 | 31982.42 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 43654.3 | 45256.3 | 47258.8 | 47659.3 | 48260.1 | 48860.8 | 49862.1 |
| | | G202_fl.wt.cre2 | 46272.49 | 48994.41 | 50355.36 | 50355.36 | 50525.48 | 52396.79 | 55118.71 |
| | | G202_fl.wt.cre3 | 55586.5 | 57379.7 | 60069.3 | 62041.8 | 63476.3 | 67779.7 | 68138.3 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 18500.21 | 18968.57 | 18968.57 | 18968.57 | 19202.75 | 19436.93 | 19436.93 |
| | | G184_fl.fl.cre1 | 21408.88 | 21408.88 | 21841.38 | 22057.63 | 22057.63 | 22057.63 | 22490.13 |
| | | G184_fl.fl.cre1 | 32714.84 | 33203.13 | 33691.41 | 33691.41 | 34179.69 | 34179.69 | 34667.97 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 52665.5 | 53266.3 | 55068.5 | 56270 | 56870.8 | 57271.3 | 57471.5 |
| | | G202_fl.wt.cre2 | 55458.95 | 56990.02 | 58350.98 | 60392.41 | 60392.41 | 60732.65 | 60902.77 |
| | | G202_fl.wt.cre3 | 71186.6 | 72800.4 | 73338.4 | 77641.9 | 77641.9 | 77641.9 | 77821.2 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 19436.93 | 19436.93 | 19671.11 | 19905.29 | 19905.29 | 19905.29 | 19905.29 |
| | | G184_fl.fl.cre1 | 22490.13 | 22706.39 | 22706.39 | 22922.64 | 23138.89 | 23138.89 | 23571.39 |
| | | G184_fl.fl.cre1 | 35400.39 | 35644.53 | 15888.67 | 35888.67 | 36132.81 | 36376.95 | 36865.23 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 59474 | 60074.8 | 60074.8 | 62077.3 | 62878.2 | 67684.2 | 68285 |
| | | G202_fl.wt.cre2 | 62944.2 | 67197.19 | 68217.9 | 70769.7 | 71790.41 | 71960.53 | 72641.01 |
| | | G202_fl.wt.cre3 | 78000.5 | 78538.4 | 78538.4 | 80152.2 | 80869.5 | 83559.1 | 86966.1 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 19905.29 | 20139.47 | 20139.47 | 20139.47 | 20373.65 | 20607.83 | 20842.01 |
| | | G184_fl.fl.cre1 | 24003.89 | 24003.89 | 24003.89 | 24436.4 | 24652.65 | 24868.9 | 25517.65 |
| | | G184_fl.fl.cre1 | 37109.38 | 37353.52 | 37841.8 | 37841.8 | 38330.08 | 38330.08 | 38330.08 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 68485.2 | 74492.7 | 77496.4 | 79298.7 | 86908.2 | 94317.4 | 99323.6 |
| | | G202_fl.wt.cre2 | 73321.49 | 77404.36 | 78254.95 | 92374.87 | 94926.66 | 96457.74 | 101051 |
| | | G202_fl.wt.cre3 | 87324.7 | 94497.1 | 95035.1 | 96828.2 | 100235 | 103283 | 103283 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 21076.19 | 21076.19 | 21076.19 | 21310.37 | 21778.73 | 22247.09 | 22481.27 |
| | | G184_fl.fl.cre1 | 25517.65 | 25733.9 | 25950.15 | 25950.15 | 26166.41 | 26382.66 | 26598.91 |
| | | G184_fl.fl.cre1 | 38818.36 | 38818.36 | 39794.92 | 39794.92 | 40039.06 | 40283.2 | 40527.34 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 106733 | 107734 | 108135 | 108936 | 125957 | 127358 | 128159 |
| | | G202_fl.wt.cre2 | 116361.7 | .120614. | 120614.7 | 139498 | 146813.1 | 147153.3 | 154978.8 |
| | | G202_fl.wt.cre3 | 104001 | 106870 | 107766 | 108842 | 110456 | 110635 | 117628 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 22481.27 | 22481.27 | 22715.45 | 22715.45 | 22715.45 | 22949.63 | 23886.35 |
| | | G184_fl.fl.cre1 | 26815.16 | 27031.41 | 27463.91 | 27463.91 | 27463.91 | 27896.42 | 28328.92 |
| | | G184_fl.fl.cre1 | 40527.34 | 40771.48 | 40771.48 | 41015.63 | 41015.63 | 41259.77 | 41748.05 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 141176 | 150788 | 182227 | 193841 | 200650 | 202051 | 207859 |
| | | G202_fl.wt.cre2 | 169949.3 | 171310.3 | 201251.3 | 206354.9 | 252287.2 | 269469.2 | 275253.3 |
| | | G202_fl.wt.cre3 | 126056 | 127490 | 138249 | 143987 | 144884 | 146677 | 147753 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 23886.35 | 24120.53 | 24354.7 | 24588.88 | 24823.06 | 25525.6 | 25525.6 |
| | | G184_fl.fl.cre1 | 28545.17 | 28545.17 | 28545.17 | 29842.68 | 30275.18 | 30275.18 | 30707.68 |
| | | G184_fl.fl.cre1 | 42236.33 | 42968.75 | 43457.03 | 43945.31 | 44677.73 | 44677.73 | 45410.16 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | 214467 | 283953 | 377870 | 390085 | 462776 | 4580500 | |
| | | G202_fl.wt.cre2 | 278485.6 | 296518.2 | 302982.8 | 318803.9 | 337176.8 | 347383.9 | 361844.1 |
| | | G202_fl.wt.cre3 | 148470 | 156360 | 176263 | 179849 | 206387 | 207105 | 223781 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 25759.78 | 25993.96 | 26930.68 | 27164.86 | 27399.04 | 27399.04 | 27867.4 |
| | | G184_fl.fl.cre1 | 30923.93 | 30923.93 | 31356.44 | 31788.94 | 32005.19 | 32005.19 | 32221.44 |
| | | G184_fl.fl.cre1 | 45654.3 | 46386.72 | 46386.72 | 46630.86 | 46630.86 | 46875 | 46875 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | 458471.9 | 526179.5 | 3963443 | | | | |
| | | G202_fl.wt.cre3 | 228622 | 310029 | 337464 | 337823 | 344816 | 344995 | 394844 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 28101.58 | 28335.76 | 28335.76 | 28569.94 | 28569.94 | 28804.12 | 29038.3 |
| | | G184_fl.fl.cre1 | 32870.2 | 33086.45 | 33302.7 | 33302.7 | 33518.95 | 34167.7 | 34383.95 |
| | | G184_fl.fl.cre1 | 47119.14 | 47363.28 | 47363.28 | 47607.42 | 47851.56 | 48339.84 | 48583.98 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | 413492 | 445230 | 471768 | 472486 | 568955 | 571824 | 646597 |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 29038.3 | 29272.48 | 29506.66 | 29740.84 | 29975.02 | 29975.02 | 30209.2 |
| | | G184_fl.fl.cre1 | 34816.46 | 35897.71 | 35897.71 | 36330.22 | 36978.97 | 36978.97 | 36978.97 |
| | | G184_fl.fl.cre1 | 48828.13 | 48828.13 | 49072.27 | 49316.41 | 50537.11 | 50781.25 | 51513.67 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | 4573697 | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 30209.2 | 30677.56 | 31614.28 | 31848.46 | 31848.46 | 32316.82 | 32316.82 |
| | | G184_fl.fl.cre1 | 37195.22 | 37627.72 | 38708.98 | 38925.23 | 39357.73 | 39790.24 | 40006.49 |
| | | G184_fl.fl.cre1 | 52978.52 | 53466.8 | 53710.94 | 56640.63 | 57373.05 | 57373.05 | 57861.33 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 32316.82 | 32316.82 | 33019.36 | 33253.54 | 33487.72 | 33487.72 | 33956.08 |
| | | G184_fl.fl.cre1 | 40006.49 | 40222.74 | 40222.74 | 40222.74 | 40222.74 | 40438.99 | 40438.99 |
| | | G184_fl.fl.cre1 | 58105.47 | 59082.03 | 59082.03 | 59326.17 | 59326.17 | 60546.88 | 61279.3 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 34424.44 | 34658.62 | 34658.62 | 34658.62 | 35126.98 | 35361.16 | 35361.16 |
| | | G184_fl.fl.cre1 | 40655.24 | 41304 | 41736.5 | 41952.75 | 42169 | 42169 | 42169 |
| | | G184_fl.fl.cre1 | 61523.44 | 62744.14 | 62988.28 | 63232.42 | 63232.42 | 63720.7 | 63964.84 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 35595.34 | 35829.52 | 35829.52 | 35829.52 | 36063.7 | 36532.06 | 36766.24 |
| | | G184_fl.fl.cre1 | 42385.25 | 43034.01 | 43250.26 | 43250.26 | 43682.76 | 43899.01 | 44331.51 |
| | | G184_fl.fl.cre1 | 63964.84 | 64208.98 | 65429.69 | 65673.83 | 66162.11 | 66406.25 | 67626.95 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 37468.78 | 37702.96 | 38171.32 | 38405.5 | 38405.5 | 38405.5 | 38873.86 |
| | | G184_fl.fl.cre1 | 44980.27 | 44980.27 | 45196.52 | 46494.03 | 46494.03 | 46926.53 | 46926.53 |
| | | G184_fl.fl.cre1 | 68847.66 | 68847.66 | 69824.22 | 73242.19 | 73974.61 | 75439.45 | 75683.59 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 39108.04 | 39108.04 | 39342.22 | 39342.22 | 39576.4 | 39576.4 | 39810.58 |
| | | G184_fl.fl.cre1 | 47359.03 | 47359.03 | 47791.53 | 47791.53 | 48440.29 | 49089.04 | 49305.29 |
| | | G184_fl.fl.cre1 | 76660.16 | 76904.3 | 78857.42 | 78857.42 | 79833.98 | 79833.98 | 80810.55 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 40044.75 | 40278.93 | 40278.93 | 40981.47 | 40981.47 | 41215.65 | 41684.01 |
| | | G184_fl.fl.cre1 | 50819.05 | 51251.55 | 51467.81 | 51467.81 | 52116.56 | 53197.82 | 53197.82 |
| | | G184_fl.fl.cre1 | 81298.83 | 82519.53 | 82763.67 | 83251.95 | 83251.95 | 83496.09 | 84228.52 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 42152.37 | 42152.37 | 42386.55 | 42386.55 | 42854.91 | 42854.91 | 42854.91 |
| | | G184_fl.fl.cre1 | 53414.07 | 53630.32 | 53846.57 | 53846.57 | 54279.07 | 54495.32 | 54711.58 |
| | | G184_fl.fl.cre1 | 84472.66 | 84716.8 | 84960.94 | 87402.34 | 87890.63 | 88378.91 | 88867.19 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 43557.45 | 43791.63 | 44962.53 | 45196.71 | 46367.61 | 46601.79 | 46601.79 |
| | | G184_fl.fl.cre1 | 54927.83 | 54927.83 | 55576.58 | 55792.83 | 56657.84 | 56874.09 | 56874.09 |
| | | G184_fl.fl.cre1 | 88867.19 | 89355.47 | 89599.61 | 90087.89 | 90332.03 | 90332.03 | 90576.17 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 46835.97 | 47070.15 | 47070.15 | 47070.15 | 47070.15 | 47070.15 | 47538.51 |
| | | G184_fl.fl.cre1 | 56874.09 | 58171.6 | 58171.6 | 58387.85 | 59469.1 | 59685.35 | 59685.35 |
| | | G184_fl.fl.cre1 | 92041.02 | 93505.86 | 93750 | 93750 | 93994.14 | 94970.7 | 95703.13 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 47772.69 | 47772.69 | 47772.69 | 48006.87 | 48475.23 | 48709.41 | 49646.13 |
| | | G184_fl.fl.cre1 | 59901.61 | 60117.86 | 60117.86 | 60117.86 | 60550.36 | 60550.36 | 60982.86 |
| | | G184_fl.fl.cre1 | 96191.41 | 96923.83 | 97900.39 | 98388.67 | 101806.6 | 104736.3 | 106445.3 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 49880.31 | 50582.85 | 50582.85 | 50817.03 | 51285.39 | 51987.93 | 52456.29 |
| | | G184_fl.fl.cre1 | 60982.86 | 62712.87 | 63145.38 | 63577.88 | 64010.38 | 64010.38 | 64226.63 |
| | | G184_fl.fl.cre1 | 107421.9 | 107910.2 | 108642.6 | 108886.7 | 109130.9 | 109375 | 110351.6 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 52690.47 | 52924.65 | 53627.19 | 53627.19 | 54563.91 | 54563.91 | 55968.98 |
| | | G184_fl.fl.cre1 | 64442.88 | 64659.13 | 64659.13 | 64659.13 | 66172.89 | 66172.89 | 66389.14 |
| | | G184_fl.fl.cre1 | 110839.8 | 111084 | 111084 | 112304.7 | 112304.7 | 112548.8 | 113281.3 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 55968.98 | 57139.88 | 57608.24 | 58310.78 | 58310.78 | 59247.5 | 59481.68 |
| | | G184_fl.fl.cre1 | 66605.4 | 66821.65 | 66821.65 | 67037.9 | 68119.15 | 69416.66 | 69849.17 |
| | | G184_fl.fl.cre1 | 113281.3 | 113769.5 | 114502 | 114502 | 114746.1 | 114746.1 | 114746.1 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 59481.68 | 59715.86 | 59715.86 | 61355.12 | 61823.48 | 61823.48 | 62057.66 |
| | | G184_fl.fl.cre1 | 69849.17 | 70281.67 | 71362.92 | 72011.68 | 72227.93 | 72444.18 | 72660.43 |
| | | G184_fl.fl.cre1 | 115234.4 | 117187.5 | 117431.6 | 119384.8 | 119628.9 | 120361.3 | 121826.2 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 62057.66 | 62057.66 | 62291.84 | 62994.38 | 63462.74 | 63462.74 | 63931.1 |
| | | G184_fl.fl.cre1 | 72876.68 | 73957.94 | 75255.45 | 75687.95 | 76120.45 | 76552.96 | 77634.21 |
| | | G184_fl.fl.cre1 | 124023.4 | 124267.6 | 125732.4 | 128173.8 | 129638.7 | 130371.1 | 131103.5 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 64165.28 | 65336.18 | 66741.26 | 66741.26 | 66975.44 | 67443.8 | 67677.98 |
| | | G184_fl.fl.cre1 | 77634.21 | 77634.21 | 77850.46 | 78066.71 | 78499.22 | 78715.47 | 78931.72 |
| | | G184_fl.fl.cre1 | 132812.5 | 133789.1 | 136718.8 | 136962.9 | 137451.2 | 138183.6 | 141845.7 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 68380.52 | 68380.52 | 68614.7 | 68848.88 | 69551.42 | 69785.6 | 70956.5 |
| | | G184_fl.fl.cre1 | 79147.97 | 79796.72 | 80229.23 | 80877.98 | 81310.48 | 82175.40 | 82607.99 |
| | | G184_fl.fl.cre1 | 142334 | 143310.5 | 146972.7 | 148193.4 | 150634.8 | 151367.2 | 152099.6 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 71659.03 | 72829.93 | 73064.11 | 73298.29 | 73766.65 | 74703.37 | 74703.37 |
| | | G184_fl.fl.cre1 | 83473 | 83689.25 | 86068.01 | 86500.51 | 86500.51 | 87365.52 | 87581.77 |
| | | G184_fl.fl.cre1 | 152832 | 156250 | 157714.8 | 159423.8 | 159912.1 | 161132.8 | 163818.4 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 75874.27 | 76810.99 | 77279.35 | 77513.53 | 77747.71 | 79152.79 | 81026.23 |
| | | G184_fl.fl.cre1 | 88230.52 | 88663.03 | 90176.79 | 90609.29 | 90825.54 | 92123.05 | 92988.05 |
| | | G184_fl.fl.cre1 | 165283.2 | 167724.6 | 168212.9 | 173095.7 | 173095.7 | 174804.7 | 175048.8 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 81026.23 | 82197.13 | 82197.13 | 84538.93 | 84773.11 | 85475.65 | 85944.01 |
| | | G184_fl.fl.cre1 | 93204.3 | 93204.3 | 93420.56 | 94069.31 | 94285.56 | 94285.56 | 94718.06 |
| | | G184_fl.fl.cre1 | 176025.4 | 183593.8 | 189453.1 | 193115.2 | 193847.7 | 195312.5 | 203125 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 86646.55 | 86880.72 | 87349.08 | 88285.8 | 88988.34 | 91798.5 | 92735.22 |
| | | G184_fl.fl.cre1 | 94718.06 | 95366.82 | 97745.58 | 99043.09 | 99475.59 | 100124.3 | 102935.6 |
| | | G184_fl.fl.cre1 | 204101.6 | 204101.6 | 218994.1 | 221679.7 | 225341.8 | 227294.9 | 230224.6 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 94140.3 | 95311.2 | 95311.2 | 95545.38 | 95779.56 | 96716.28 | 97418.82 |
| | | G184_fl.fl.cre1 | 102935.6 | 103800.6 | 104881.9 | 104881.9 | 105530.6 | 108558.1 | 108558.1 |
| | | G184_fl.fl.cre1 | 234375 | 234863.3 | 239746.1 | 239990.2 | 241455.1 | 246582 | 254394.5 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 97887.18 | 98121.36 | 101165.7 | 103975.9 | 105146.8 | 105615.1 | 107020.2 |
| | | G184_fl.fl.cre1 | 110071.9 | 112018.2 | 112450.7 | 112666.9 | 115478.2 | 116343.2 | 116775.7 |
| | | G184_fl.fl.cre1 | 258544.9 | 260253.9 | 268554.7 | 276367.2 | 276855.5 | 278564.5 | 281738.3 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 107956.9 | 108659.5 | 108659.5 | 109127.8 | 109362 | 110298.7 | 111469.6 |
| | | G184_fl.fl.cre1 | 118289.5 | 118505.7 | 118938.2 | 119587 | 121965.7 | 122830.7 | 125425.7 |
| | | G184_fl.fl.cre1 | 282226.6 | 286621.1 | 293701.2 | 310058.6 | 331543 | 339599.6 | 352539.1 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 111469.6 | 111938 | 112406.3 | 112406.3 | 114513.9 | 115450.7 | 115450.7 |
| | | G184_fl.fl.cre1 | 125858.2 | 126074.5 | 126290.8 | 126723.3 | 127804.5 | 128669.5 | 128885.8 |
| | | G184_fl.fl.cre1 | 379882.8 | 387207 | 391113.3 | 395996.1 | 399902.3 | 427734.4 | 438476.6 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 117558.3 | 118729.2 | 119665.9 | 119665.9 | 122710.2 | 123881.1 | 124349.5 |
| | | G184_fl.fl.cre1 | 129318.3 | 129967 | 133643.3 | 134724.6 | 135589.6 | 136022.1 | 136454.6 |
| | | G184_fl.fl.cre1 | 438964.8 | 489990.2 | 506835.9 | 536621.1 | 564209 | 575439.5 | 576171.9 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 125052 | 125754.6 | 125988.8 | 127628 | 128096.4 | 130438.2 | 134887.6 |
| | | G184_fl.fl.cre1 | 136887.1 | 141644.6 | 145104.6 | 146618.4 | 147050.9 | 150943.4 | 151375.9 |
| | | G184_fl.fl.cre1 | 652832 | 720947.3 | 758789.1 | 776123 | 795898.4 | 838867.2 | 868652.3 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 137463.6 | 153387.8 | 153387.8 | 156198 | 161584.1 | 161818.3 | 162520.8 |
| | | G184_fl.fl.cre1 | 151808.4 | 152889.7 | 155268.4 | 156349.7 | 156998.4 | 158079.7 | 160242.2 |
| | | G184_fl.fl.cre1 | 1003174 | 1069824 | 1119385 | 1288086 | 1483398 | 1751221 | 2100586 |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 163691.7 | 165565.2 | 167204.4 | 175634.9 | 175634.9 | 175869.1 | 178679.2 |
| | | G184_fl.fl.cre1 | 162621 | 164999.7 | 166081 | 168027.2 | 171919.8 | 188354.9 | 190517.4 |
| | | G184_fl.fl.cre1 | | | | | | | |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 180552.7 | 182426.1 | 184767.9 | 191793.3 | 193900.9 | 194135.1 | 197882 |
| | | G184_fl.fl.cre1 | 193761.2 | 200032.4 | 202627.5 | 203708.7 | 213223.8 | 213223.8 | 216683.8 |
| | | G184_fl.fl.cre1 | | | | | | | |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 203736.5 | 204204.8 | 212401.1 | 222002.5 | 225281 | 228559.5 | 235584.9 |
| | | G184_fl.fl.cre1 | 221225.1 | 2242526 | 229226.4 | 253014 | 253662.8 | 259501.5 | 259501.5 |
| | | G184_fl.fl.cre1 | | | | | | | |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 236990 | 246591.4 | 250104.1 | 262984 | 283357.6 | 284996.9 | 293427.4 |
| | | G184_fl.fl.cre1 | 264042.8 | 269016.6 | 281342.9 | 281991.7 | 302751.8 | 314213.1 | 322646.9 |
| | | G184_fl.fl.cre1 | | | | | | | |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 299984.4 | 302560.4 | 322699.8 | 322934 | 325275.8 | 325510 | 326212.5 |
| | | G184_fl.fl.cre1 | 327836.9 | 347732.1 | 359193.4 | 382981 | 388171.1 | 391414.8 | 398767.4 |
| | | G184_fl.fl.cre1 | | | | | | | |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 340497.5 | 340965.9 | 352909 | 364852.2 | 375624.5 | 377732.1 | 392251.2 |
| | | G184_fl.fl.cre1 | 403524.9 | 411742.4 | 413472.5 | 452830.2 | 456939 | 463642.8 | 469697.8 |
| | | G184_fl.fl.cre1 | | | | | | | |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 453372.2 | 458524.1 | 477492.7 | 490138.4 | 500442.3 | 511448.8 | 511683 |
| | | G184_fl.fl.cre1 | 493701.7 | 534356.9 | 559658.3 | 594691 | 597718.5 | 736984.4 | 742606.9 |
| | | G184_fl.fl.cre1 | | | | | | | |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | | | | |
|---|---|---|---|---|---|---|---|---|---|
| fl/wt Cre− | G202 | G202_fl.wt.cre1 | | | | | | | |
| | | G202_fl.wt.cre2 | | | | | | | |
| | | G202_fl.wt.cre3 | | | | | | | |
| fl.wt.cre+ | G251 | G251_fl.wt.cre1 | | | | | | | |
| | | G251_fl.wt.cre2 | | | | | | | |
| | | G251_fl.wt.cre3 | | | | | | | |
| fl/fl Cre− | G184 | G184_fl.fl.cre1 | 547980.8 | 598563.7 | 607696.7 | 681463.4 | 716590.3 | 763426.3 | 828528.3 |
| | | G184_fl.fl.cre1 | 749094.4 | 751905.7 | 764664.5 | 780883.4 | 818727.4 | 897010.3 | 1127750 |
| | | G184_fl.fl.cre1 | | | | | | | |
| fl/fl Cre+ | G209 | G209_fl.fl.cre1 | | | | | | | |
| | | G209_fl.fl.cre1 | | | | | | | |
| | | G209_fl.fl.cre1 | | | | | | | |

A. Sizes of distinct metastatic nodes (um\hat{ }2) in the lung samples used for analysis

| | | | | | | |
|---|---|---|---|---|---|---|
| fl/wt Cre− | G202 | G202_fl.wt.cre1 | | | | |
| | | G202_fl.wt.cre2 | | | | |
| | | G202_fl.wt.cre3 | | | | |
| fl.wt.cre+ | G251 | G251_fl.wt.cre1 | | | | |
| | | G251_fl.wt.cre2 | | | | |
| | | G251_fl.wt.cre3 | | | | |
| fl/fl Cre− | G184 | G184_fl.fl.cre1 | 846091.8 | 988004.8 | 988473.1 | 1460580 |
| | | G184_fl.fl.cre1 | 1161486 | 1172731 | | |
| | | G184_fl.fl.cre1 | | | | |
| fl/fl Cre+ | G209 | G209_fl.fl.cre1 | | | | |
| | | G209_fl.fl.cre1 | | | | |
| | | G209_fl.fl.cre1 | | | | |

B. Goodnes-off fit analysis of the frequency distribution of pulmany node metastatses size
(See details regading to mathematical model and paramerization in Methods and FIG. 6.
Frequency Distribution: f (x) = y0 + a*exp(−0.5*(ln(x/x0)/b)\hat{ }2)

| | Datasets | | | | | | | |
|---|---|---|---|---|---|---|---|---|
| | G202_fl/wt_heterozygous_Cre− | | | | G251_fl/wt_heterozygous_Cre+ | | | |
| | Rsqr = 0.830 Adj Rsqr = 0.827 | | | | Rsqr = 0.7 Adj Rsqr = 0.703 | | | |
| | Standard Error of Estimate = 1.7893 | | | | Standard Error of Estimate = 1.1800 | | | |
| | Coefficient Std. Error | t-test | P | | Coefficien Std. Error | t-test | P | |
| a | 14.48 | 0.590 | 24.6 | <0.0001 | 6.13 | 0.454 | 13.5 | <0.0001 |
| b | 0.92 | 0.049 | 18.7 | <0.0001 | 0.87 | 0.089 | 9.8 | <0.0001 |
| x0 | 7.69 | 0.429 | 17.9 | <0.0001 | 6.30 | 0.582 | 10.8 | <0.0001 |
| y0 | 1.25 | 0.213 | 5.9 | <0.0001 | 1.09 | 0.202 | 5.4 | <0.0001 |

B. Goodnes-off fit analysis of the frequency distribution of pulmany node metastatses size
(See details regading to mathematical model and paramerization in Methods and FIG. 6.
Frequency Distribution: f (x) = y0 + a*exp(−0.5*(ln(x/x0)/b)\hat{ }2)

| | Datasets | | | | | | | |
|---|---|---|---|---|---|---|---|---|
| | G184.fl/fl_homozygous_Cre− | | | | G209_fl/fl_homozygous_fl/fl_Cre+ | | | |
| | G184.fl/fl Adj Rsqr = 0.8369 | | | | Rsqr = 0.5. Adj Rsqr = 0.498 | | | |
| | Standard Error of Estimate = 2.2186 | | | | Standard Error of Estimate = 1.1022 | | | |
| | Coefficient Std. Error | t-test | P | | Coefficien Std. Error | t--test | P | |
| a | 20.50 | 0.590 | 34.8 | <0.0001 | 4.72 | 0.770 | 6.1 | <0.0001 |
| b | 1.19 | 0.046 | 25.8 | <0.0001 | 1.87 | 0.471 | 4.0 | 0.0004 |
| x0 | 8.59 | 0.489 | 17.6 | <0.0001 | 1.26 | 0.992 | 1.3 | 0.2122 |
| y0 | 1.07 | 0.208 | 5.2 | <0.0001 | 0 | 0 | 0 | 0 |

| Analysis of Variance: | DF | SS | MS | F | P | DF | SS | MS | F | P |
|---|---|---|---|---|---|---|---|---|---|---|
| Regression | 3 | 2313.2564 | 771.0855 | 240.8561 | <0.0001 | 3 | 284.5746 | 94.8582 | 68.123 | <0.0001 |

| Analysis of Variance: | DF | SS | MS | F | P | DF | SS | MS | F | P |
|---|---|---|---|---|---|---|---|---|---|---|
| Regression | 3 | 7594.147 | 2531.382 | 514.2695 | <0.0001 | 2 | 42.3404 | 21.1702 | 17.4266 | <0.0001 |

TABLE 11
Primary tumor size kinetic data. The sample are ranked-
order by time and cumulative tumor size at week 6.

| | fl/fl Cre− | | | | | | | | |
|---|---|---|---|---|---|---|---|---|---|
| | Animal | w 0 | w 1 | w 2 | w 3 | w 4 | w 5 | w 6 | kinetic type |
| 1 | G260 | 2 | 2 | 1 | 2 | 1 | 1 | 2 | d/slow |
| 2 | G256 | 1 | 6 | 1 | 2 | 2 | 2 | 7 | d/slow |
| 3 | G335 | 2 | 4 | 9 | 5 | 5 | 4 | [symbol] | d/slow |
| 4 | G246 | 1 | 9 | 6 | 16 | 25 | 27 | 27 | d/slow |
| 5 | G281 | 2 | 2 | 2 | 3 | 10 | 6 | [symbol] | slow |
| 6 | G278 | 1 | 3 | 3 | 9 | 22 | 34 | [symbol] | slow |
| 7 | G184a | 1 | 1 | 3 | 3 | 9 | 15 | [symbol] | slow |
| 8 | G181 | 2 | 6 | 6 | 15 | 21 | 133 | [symbol] | slow |
| 9 | G113 | 1 | 1 | 28 | 213 | 182 | 376 | 150 | [symbol] |
| 10 | G227 | 1 | 8 | 50 | 32 | 52 | 152 | 213 | [symbol] |
| 11 | G111 | 1 | 8 | 41 | 53 | 110 | 100 | 237 | [symbol] |
| 12 | G208 | 4 | 15 | 54 | 76 | 122 | 230 | 378 | [symbol] |
| 13 | G284 | 6 | 1 | 9 | 5 | 57 | 148 | 403 | [symbol] |
| | Total | 20 | 64 | 211 | 432 | 617 | 1227 | 1687 | |

| | fl/fl Cre+ | | | | | | | | |
|---|---|---|---|---|---|---|---|---|---|
| | | w 0 | w 1 | w 2 | w 3 | w 4 | w 5 | w 6 | kinetic type |
| 1 | G234 | 1 | 1 | 2 | 9 | 2 | 2 | 2 | d/slow |
| 2 | G267 | 2 | 2 | 2 | 2 | 2 | 2 | 2 | d/slow |
| 3 | G269b | 1 | 1 | 1 | 1 | 1 | 3 | 4 | d/slow |
| 4 | G231 | 5 | 15 | 2 | 6 | 9 | 9 | [symbol] | slow |
| 5 | G209 | 1 | 1 | 1 | 4 | 3 | 6 | 21 | slow |
| 6 | G215 | 1 | 1 | 1 | 3 | 16 | 21 | [symbol] | slow |
| 7 | G207 | 1 | 2 | 25 | 20 | 24 | 20 | [symbol] | [symbol] |
| 8 | G269a | 1 | 1 | 6 | 7 | 9 | 150 | [symbol] | [symbol] |
| 9 | G190 | 2 | 2 | 3 | 3 | 28 | 76 | [symbol] | [symbol] |
| 10 | G140 | 1 | 2 | 67 | 85 | 123 | 302 | 370 | [symbol] |
| 11 | G125 | 145 | 151 | 190 | 371 | 250 | 437 | [symbol] | [symbol] |
| 12 | G145 | 4 | 7 | 27 | 40 | 49 | 338 | [symbol] | [symbol] |
| 13 | G174 | 4 | 7 | 5 | 20 | 359 | 531 | [symbol] | [symbol] |
| | | 165 | 190 | 329 | 569 | 876 | 1895 | 3803 | |

| | fl/wt Cre− | | | | | | | | |
|---|---|---|---|---|---|---|---|---|---|
| | Animal | w 0 | w 1 | w 2 | w 3 | w 4 | w 5 | w 6 | kinetic type |
| 1 | G249 | 1 | 1 | 9 | 8 | 5 | 17 | 8 | d/slow |
| 2 | G235 | 1 | 5 | 5 | 2 | 2 | 3 | 10 | slow |
| 3 | G223 | 2 | 2 | 2 | 7 | 10 | 21 | 20 | slow |
| 4 | G236 | 1 | 5 | 16 | 18 | 13 | 37 | [symbol] | slow |
| 5 | G288 | 2 | 1 | 2 | 3 | 1 | 14 | 120 | [symbol] |
| 6 | G202 | 1 | 2 | 20 | 3 | 53 | 108 | 199 | [symbol] |
| 7 | G232 | 14 | 14 | 23 | 83 | 105 | 201 | 347 | [symbol] |
| 8 | G184 | 1 | 35 | 45 | 85 | 689 | 629 | 625 | [symbol] |
| 9 | G144 | 49 | 54 | 110 | 188 | 302 | 487 | 870 | [symbol] |
| 10 | G128 | 1 | 1 | 89 | 284 | 377 | 599 | 1056 | [symbol] |
| 11 | G299 | 2 | 2 | 1 | 37 | 249 | 680 | 2020 | [symbol] |
| | Total | 72 | 120 | 322 | 717 | 1806 | 2796 | 5325 | |

| | fl/wt Cre+ | | | | | | | | |
|---|---|---|---|---|---|---|---|---|---|
| | Animal | w 0 | w 1 | w 2 | w 3 | w 4 | w 5 | w 6 | kinetic type |
| 1 | G129 | 0.5 | 0.5 | 11.3 | 0.5 | 0.5 | 0.5 | 0.5 | d/slow |
| 2 | G287 | 1.5 | 1.0 | 1.5 | 1.5 | 3.0 | 10.0 | 12.5 | slow |
| 3 | G257 | 1.5 | 1.5 | 1.0 | 1.5 | 0.5 | 1.0 | [symbol] | slow |
| 4 | GG251 | 1.0 | 1.5 | 9.0 | 6.0 | 10.0 | 20.0 | [symbol] | slow |
| 5 | G282 | 0.5 | 1.0 | 2.0 | 2.0 | 2.0 | 7.5 | 28.8 | slow |
| 6 | G165 | 0.5 | 1.2 | 4.8 | 13.3 | 0.5 | 4.7 | 37.2 | slow |
| 7 | G185 | 0.5 | 1.0 | 2.0 | 2.5 | 4.4 | 2.5 | 54.8 | slow |
| 8 | G199 | 0.5 | 1.0 | 1.5 | 1.5 | 1.5 | 16.9 | [symbol] | slow |
| 9 | G292 | 1.5 | 1.0 | 2.0 | 7.6 | 8.8 | 53.4 | 78.0 | slow |
| 10 | G164 | 1.0 | 0.5 | 6.4 | 18.8 | 32.6 | 60.0 | 286.5 | [symbol] |
| 11 | G271 | 1.5 | 5.5 | 6.0 | 32.6 | 80.6 | 373.1 | 615.6 | [symbol] |
| | Total | 10.5 | 15.7 | 47.5 | 87.7 | 144.3 | 549.6 | 1231.3 | |

Notice: 85 mm\hat{ }3 is empirical cut-off value for the comparative analysis of dormancy/slow-rate (d/slow) and fast- rate growing tumors. Red color indicates the tumor volume more than 9 mm\hat{ }3. [symbol]
indicates data missing or illegible when filed

What is claimed is:

1. A method of determining the personalized risk of metastasis of breast cancer and the risk of survival in a subject who has or had breast cancer, the method comprising:
(a) obtaining a tissue sample from the subject;
(b) measuring gene expression profiles of ABI1, BRK1, WASF3, CYFIP1, CYFIP2, RAC1 and NDEL1 genes in the tissue sample;
(c) comparing the gene expression profiles of above-mentioned seven genes in the tissue sample from a high-risk primary tumor from the subject with metastatic or death outcomes with the expression profiles of these genes from a low-risk primary tumor, thereby comprising a seven-gene prognostic signature;
(d) determining the risk of the subject using data-driven grouping (DDg) methods, gene expression data and statistically weighted voting grouping (SVWg) algorithms; and
(e) stratifying the subject in a high, moderate, or low risk group based on the determining step.

2. The method of claim 1, wherein a computer readable medium having stored thereon a computer program which, when executed by a computer system operably connected to a gene or protein expression assay system configured to measure an expression signal of a plurality of genes in a tissue sample obtained from a subject, causes the computer system to perform a method of calculating a cut-off value by a method comprising:
(a) fitting said measured expression signal of ABI1, BRK1, WASF3, CYFIP1, CYFIP2, the WAVE complex members, and also RAC1 and NDEL1 as independent variables, interpreting the expression signals and calculating a p-value using a data-driven grouping (DDg) method;
(b) constructing a seven-gene prognostic signature using a statistically weighted voting grouping (SVWg) algorithm and input data provided by the data-driven grouping (DDg) method; and,
stratifying the subject in a high, moderate, or low risk group based on the seven-gene prognostic signature.

3. The method of claim 1, wherein ABI1 is an independent prognostic metastatic biomarker in breast cancer.

4. The method of claim 1, wherein a metastasis is associated with ABI1 gene dose and specific gene expression aberrations in primary breast cancer tumors.

5. The method of claim 1, wherein the subject is a mammal.

6. The method of claim 5, wherein the mammal is a human.

7. The method of claim 1, wherein the tissue sample is a mammary gland tissue sample or a breast cancer tissue sample or its derivatives found in the human body at any stage of the disease.

8. The method of claim 1, wherein ABI1 gene is highly or abnormally expressed, thereby representing a therapeutic target, as defined in a metastatic ABI1/PyMT mouse model system, wherein ABI1 gene downregulation reduces metastatic burden in lungs.
